(19) United States
(12) Patent Application Publication
(10) Pub. No.: US 2005/0042716 A1
(43) Pub. Date: Feb. 24, 2005

(54) Allergen inactivating method, allergen inactivating filter, air treating apparatus, virus inactivating agent, virus inactivating method, virus inactivating filter, air conditoning unit and air conditioner

(76) Inventors: Yuji Nakajima, Yokohama-shi (JP); Daisuke Tanaka, Nagoya-shi (JP); Katsuhiro Hashitsume, Nagoya-shi (JP); Naokazu Takeuchi, Nagoya-shi (JP); Susumu Kojima, Nagoya-shi (JP); Masaki Bessyo, Nagoya-shi (JP); Yuuri Tsuboi, Osaka-shi (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 10/768,965

(22) Filed: Feb. 2, 2004

(30) Foreign Application Priority Data
Aug. 19, 2003 (JP) 2003-207883
Oct. 7, 2003 (JP) 2003-348670

(51) Int. Cl. C12P 21/06

(52) U.S. Cl. 435/68.1

(57) ABSTRACT

The invention provides an allergen inactivating method by maintaining the allergens under a condition in which any one selected from the group consisting of heat, an alkali, an acid and an enzyme exists.

### CROSS-REFERENCE TO RELATED APPLICATIONS

This application is based upon and claims the benefit of priority from the prior Japanese Patent Applications No. 2003-207883, filed Aug. 19, 2003; and No. 2003-348670, filed Oct. 7, 2003, the entire contents of both of which are incorporated herein by reference.

### BACKGROUND OF THE INVENTION

1. Field of the Invention

The invention relates to an allergen inactivating method for inactivating allergens. The invention also relates to an allergen inactivating filter or a virus inactivating filter that traps allergens mainly comprising living organisms derived proteins as allergy causing substances for denaturation and decomposition of allergens. The invention further relates to an air treating apparatus such as an air conditioner integrated with the filter, a dehumidifier, a humidifier, a ventilator and an air cleaner, public dust removing facilities provided with the air treating apparatus, a virus inactivating agent, an air conditioning unit, and an apparatus for cleaning air in a given space such as in the room and passenger's cabin including an air conditioner integrated with the air conditioning unit. In addition, the invention relates to an apparatus for cleaning air that flows in and out of a given space such as a vacuum cleaner, a bedclothes drier, a mask for human use, surgical and medical clean room facilities, an air treating apparatus provided in a biological terrorism countermeasure facility and a small size air treating apparatus attached to a biological terrorism protecting clothes.

2. Description of the Related Art

As are well known, the problems of allergy have been highlighted in recent years due to increase of highly hermetic houses. Pollens are popular as allergens. However, allergy caused by carcass and secretions of harmful insects such as mites and cockroaches have became serious problems, and various products have been supplied in the world as counter-measures thereof. Nevertheless, technologies for identifying and eliminating allergy causing substances (allergens) have not been established yet, and only cleaning tools (cleaning machine and mops) for collectively treating house dusts have are commercially available.

While allergy is caused by direct contact of allergens on the skin, most of allergy is caused by inhalation of allergens floating in air. Accordingly, it may be conjectured that the symptom of allergy may be alleviated by removing allergens in air. In particular, biologically induced allergens such as mite and pollens allergens are mainly composed of proteins. Therefore, allergens may be inactivated by denaturing the allergen proteins.

The methods commonly known for denaturing proteins are chemical denaturation using an acid or alkali, and physical denaturation by high temperatures. It is also well known that proteins are decomposed using protein decomposing enzymes (proteases).

The following methods for excluding allergens have been conventionally known. For example, intake of pollens represented by pollens of cedar into the body is protected by wearing a mask having a fine mesh cloth that blocks pollens in outdoors. However, pollens are only trapped as a part of floating substances in air using, for example, a dust collector, and more active method for removing the pollens in residential spaces have not been used yet.

The amount of allergens derived from harmful insects represented by mites are alleviated by frequently cleaning bedclothes, floor mats and floors to thereby reduce the amount of the harmful insects. However, while the amount of allergens are temporarily reduced by these methods, a considerable amount of manpower is required for maintaining allergens in a low level since the amount of allergens increases depending on proliferation of harmful insects. While using chemicals for exterminating the harmful insects or the like may be an option, the method cannot be a sufficient measure considering the effect of the chemicals themselves on human bodies.

The amount of allergens can be automatically reduced by providing allergen removing means in air conditioning related equipment. Since mite and pollen allergens are particles, they can be trapped by using a filter. The term “trap” as used herein means to capture objects or to remove unnecessary substances. Since the trapped allergens may be scattered again, it is important to inactivate the allergens trapped by the filter with some means. However, no equipment providing such a function is not known.

Conventionally, as a method of immobilizing enzymes on a substrate, a method by a biosensor or the like is known. Jpn. Pat. Appln. KOKAI Publication No. 6-91117 has disclosed a filter having a substrate on which enzymes and antibiotics produced by mucus bacteria are immobilized. WO 98/04334 (patent reference) has disclosed an air cleaning filter having enzymes immobilized on the surface of a carrier of an air cleaning filter, whereby microorganisms that have been difficult to remove with conventional air cleaning filters are directly cleaned by sterilization while removing the microorganisms trapped on the filter by sterilization or disinfection.

However, it has not been proposed to provide a filter comprising enzymes having allergen inactivating functions for inactivating floating allergens.

Infectious diseases caused by viruses are being serious problems today, and spread of influenza has been a great topic every year in the media. Since influenza has been treated due to development of medicine, infection by influenza may be possibly prevented by inoculating the vaccine before it is prevailing. However, many victims could not be avoided from appearing before the development of vaccines when unknown viruses that have not been discovered yet such as SARS virus that recently caused confusions have suddenly emerged. Spreading of viruses such as smallpox viruses that have been considered to be already stamped out cannot be denied due to artificial infection by biological terrorism or accidental invasion of infection. In such cases, non-immunized persons such as those who have not been vaccinated with smallpox vaccines may be exposed to fatal dangers. Countermeasures against such unknown viruses or viruses that have been disappeared from the memory available for humankind are (1) to keep out of the area having potential dangers of infection, and (2) to avoid contact with persons who have been possibly infected. However, such measures are not practical since productivity of the entire society may be decreased. Protective clothes and devices for persons fighting against these viruses are also required.

It is usually effective for inactivating viruses to immerse the virus in alcohol or in an alkaline solution or an aqueous hypochlorous acid solution. However, such methods only temporarily inactivate the viruses. Moreover, chemicals such as acids and alkali are quite toxic to humans as well as animals, and cannot be handled without cautions.

If a method of inactivating continuous virus is developed, it is possible to develop a virus efficiently inactivating system that can be used in, for example, an air conditioner. Furnishing such air conditioning facilities may be advantageous for enabling closed spaces to be safely maintained even when the dangers of spreading of viruses occur. While the patent reference has disclosed an air cleaning filter on which enzymes are immobilized, the air cleaning filter only sterilizes microorganisms and is poor in virus removing ability since only enzymes are used.

Accordingly, a virus inactivating agent and a virus inactivating method that are applicable to an air conditioner or the like and effectively and continuously inactivate viruses have been desired. Also desired are a virus inactivating filter provided with the inactivating agent, and an air conditioner provided with the filter.

### BRIEF SUMMARY OF THE INVENTION

Accordingly, an object of the invention is to provide an allergens inactivating method for specifically excluding allergens, an allergen inactivating filter, an air treating apparatus, a virus inactivating agent, a virus inactivating method, a virus inactivating filter, an air conditioning unit and air conditioner, and facilities and home electric appliances provided with an air cleaning function.

An allergen inactivating method according to a first aspect of the present invention inactivates the allergens by maintaining the allergens under a condition in which any one selected from the group consisting of heating, an alkali, an acid and an enzyme exists.

An allergen inactivating method according to a second aspect of the present invention inactivates the allergens by maintaining the allergens under a condition in which any one selected from the group consisting of an alkali, an acid and an enzyme, and heat exist.

An allergen inactivating filter according to a third aspect of the present invention comprises a filter main body and any one of inactivating means of heat, an acid, an alkali and an enzyme having an allergen inactivating function.

An air treating apparatus according to a fourth aspect of the present invention comprises the allergen inactivating filter.

A virus inactivating agent according to a fifth aspect of the present invention comprises at least one active component selected from the group consisting of a protein denaturing agent and a protein decomposing enzyme.

A virus inactivating method according to a sixth aspect of the present invention inactivates viruses by allowing the virus to contact a solution containing the virus inactivating agent.

A virus inactivating filter according to a seventh aspect of the present invention comprises a filter which traps the virus and the virus inactivating agent adhered on the filter.

An air conditioning unit according to an eighth aspect of the present invention comprises an air suction port to suck air, a heat exchanger to cool or heat the air sucked from the suction port by heat-exchange between the air and a coolant, an air blow port to return the air heat-exchanged by the heat exchanger into a room, ventilation means for blowing the air sucked from the suction port and heat-exchange by the heat exchanger into the room from the air blow port, a virus inactivating filter which immobilizes the virus inactivating agent disposed in an inner space through which air flows, and inactivating agent activating means for maintaining the inner space in an atmosphere in which the virus inactivating agent is activated.

An air conditioner according to a ninth aspect of the present invention comprises the air conditioning unit, a second air conditioning unit having a compressor to compresses a refrigerant and a heat exchanger to heat-exchange between the refrigerant and air, and a refrigerant pipe line to connect between the two air conditioning units with each other while the refrigerant is allowed to circulate between the two air conditioning units.

### BRIEF DESCRIPTION OF THE SEVERAL VIEWS OF THE DRAWING

FIG. 1 is a view showing an example of an allergen inactivating filter according to the invention;

FIG. 2 is a view showing an example of the allergen inactivating filter according to the invention;

FIG. 3 is a view showing an example of the allergen inactivating filter according to the invention;

FIG. 4 is a schematic diagram of the result of Western blotting;

FIG. 5 is a graph showing a calibration curve obtained by Western blotting;

FIG. 6 a schematic view of mite scanning of the band obtained when the sample contains a large amount mite allergen;

FIG. 7 is a graph showing the relation between the location of the band on the mite scanning and light emission intensity;

FIG. 8 is a graph showing a calibration curve with respect to the mite allergen contained in the mite extract;

FIG. 9 is a graph showing the relation between the heat treatment time and the amount of the mite allergen in the sample;

FIG. 10 is a schematic view of the band seen in mite scanning after reaction;

FIG. 11 is a schematic view of the band seen in mite scanning after reaction;

FIG. 12 is a schematic view of the band seen in mite scanning after reaction;

FIG. 13 is a schematic view of the band seen in mite scanning after reaction;

FIG. 14 is a schematic view of the band seen in mite scanning after reaction;

FIG. 15 shows an air conditioner according to the invention having the allergen inactivating filter shown in FIG. 1 attached to the air inlet side thereof;

FIG. 16A is an entire view of an allergen inactivating filter according to Example 13 of the invention;

FIG. 16B is a partial enlarged view of FIG. 16A;

FIG. 17 is an explanatory view of major portions of an allergen inactivating filter according to Example 14 of the invention;

FIG. 18A is an explanatory view of an allergen inactivating filter according to Example 15 of the invention;

FIG. 18B is an explanatory view of another allergen inactivating filter;

FIG. 19 is an explanatory view when the filters in FIGS. 13 to 15 attached to the air conditioner;

FIG. 20A is an explanatory view of an allergen inactivating filter according to Example 16 of the invention;

FIG. 20B is an explanatory view of an allergen inactivating filter according to Example 17 of the invention;

FIG. 20C is an explanatory view of an allergen inactivating filter according to Example 18 of the invention;

FIG. 21A is a view explaining a method for feeding water to an allergen inactivating filter according to Example 19 of the invention;

FIG. 21B is an enlarged sectional view taken along line 21B-21B in FIG. 21A;

FIG. 22 is a view explaining a method for feeding water to an allergen inactivating filter according to Example 20 of the invention;

FIGS. 23A and 23B are view illustrating a rod member showing a different embodiment from the rod member used in the method for feeding water in FIG. 21;

FIG. 24A is a view explaining a method for feeding water to an allergen inactivating filter according to Example 21 of the invention;

FIG. 24B is a view along the arrow X in FIG. 24A;

FIG. 24C is a partial enlarged view of FIG. 24B;

FIG. 25 is a view explaining a method for feeding water to an allergen inactivating filter according to Example 22 of the invention;

FIGS. 26A, 26B and 26C are schematic view explaining a method for feeding water to a flat type allergen inactivating filter according to Example 23 of the invention, respectively;

FIG. 27 is a diagram showing a method for heating an allergen inactivating filter according to Example 24 of the invention;

FIG. 28A is an explanatory view when the filter is used;

FIG. 28B is an explanatory view when the filter is not used;

FIG. 28C is a view along the arrow X in FIG. 28B;

FIGS. 29A and 29B are explanatory views of an allergen inactivating filter providing an additional function according to Example 26;

FIGS. 30A and 30B are diagrams illustrating an allergen inactivating filter providing a dust removing function according to Example 15 of the invention, respectively;

FIG. 31 is a diagram of an apparatus for confirming the service life of the allergen inactivating filter according to the invention;

FIG. 32A is a diagram of another apparatus for confirming the service life of the allergen inactivating filter according to the invention;

FIG. 32B is a diagram of another apparatus for confirming the service life of the allergen inactivating filter according to the invention;

FIG. 33 is a diagram of another apparatus for confirming the service life of the allergen inactivating filter according to the invention;

FIG. 34 is a diagram of another apparatus for confirming the service life of the allergen inactivating filter according to the invention;

FIG. 35 is an explanatory view illustrating an air conditioner having the allergy inactivating filter according to the invention mounted thereon;

FIG. 36 is a graph showing the relation between the heating time and the amount of the mite allergen in the sample;

FIG. 37 is a graph showing the relation between the heating time and the amount of the mite allergen in the sample;

FIG. 38 is a graph showing the relation between the heating time and the amount of the mite allergen in the sample;

FIG. 39 is a graph showing the relation between the heating time and the amount of the mite allergen in the sample;

FIG. 40 is a schematic view showing the result of electrophoresis;

FIG. 41 is a schematic view showing the result of electrophoresis;

FIG. 42 is a schematic view showing the result of electrophoresis;

FIG. 43 is a schematic view showing the result of electrophoresis;

FIG. 44 is a schematic view showing the result of Western blotting;

FIG. 45 is a schematic view showing the result of Western blotting;

FIG. 46 is a schematic view showing the result of Western blotting;

FIG. 47 is a schematic view showing the result of Western blotting;

FIG. 48 is a schematic view showing the result of Western blotting;

FIG. 49 is a schematic view showing the result of Western blotting;

FIG. 50 is a schematic view showing the result of electrophoresis;

FIG. 51 is a schematic view showing the result of electrophoresis;

FIG. 52 is a schematic view showing the result of electrophoresis;

FIG. 53 is a schematic view showing the result of electrophoresis;

FIG. 54 is a schematic view showing the result of electrophoresis;

FIG. 55 is a schematic view showing the result of electrophoresis;

FIG. 56 is a schematic view showing the result of Western blotting;

FIG. 57 is a schematic view showing the result of Western blotting;

FIG. 58 is a graph showing the inactivation ratio of λ-phage;

FIG. 59 is a graph showing the inactivation ratio of M13 phage;

FIG. 60 is a graph showing the changes of absorbance of an E. coli suspension solution;

FIG. 61 is a graph showing the inactivation graph of λ-phage by the inactivating filter;

FIG. 62 is a graph showing the infection ability of λ-phage using the inactivating filter;

FIG. 63 is a graph showing the infection ability of M13 phage by the inactivating filter;

FIG. 64 is a graph showing the infection ability of M13 phage using the inactivating filter;

FIG. 65 shows a dry test method for fungi;

FIG. 66 shows the result of the dry test for fungi;

FIG. 67 shows the result of an wet test for fungi;

FIG. 68 is a cross section showing a first embodiment of an air conditioning indoor unit according to the invention;

FIG. 69 is a perspective view of the schematic configuration of an air conditioner according to the invention;

FIG. 70 is a refrigerator circuit of the air conditioner shown in FIG. 69;

FIG. 71 is a plan view of an example of a remote controller;

FIG. 72 is a cross section of a modification of the air conditioning indoor unit shown in FIG. 68;

FIG. 73 is a cross section of major portions showing a second embodiment of the air conditioning indoor unit according to the invention; and

FIG. 74 is a plan view showing the container in FIG. 73.

### DETAILED DESCRIPTION OF THE INVENTION

An allergen inactivating method, an allergen inactivating filter, an air treating apparatus, a virus inactivating agent, a virus inactivating method, a virus inactivating filter, an air conditioning unit and an air conditioner according to the invention will be described hereinafter.

(1) In a first allergen inactivating method of the invention, allergens are inactivated by maintaining the allergens under a condition in which any one selected from the group consisting of heat, an alkali, an acid and an enzyme exists.

1) Allergen Inactivating Method

The allergen as an allergy causing substance is mainly composed of biologically induced proteins. The invention is based on elucidation of capability of specifically and effectively inactivating the allergen by denaturing the proteins by the inventors. Accordingly, the specification of the invention is the first for reporting that the allergen ca be inactivated by denaturing the proteins. The terms “allergen” and “allergen substance” as used herein refer to substances exhibiting activities as the allergens. The allergen derived from pollens is described as the “pollen allergen”.

Pollen disease is induced by inhalation of pollens as the allergen in air. Accordingly, allergic symptoms are considered to be alleviated by blocking and/or excluding the allergens in air. On the other hand, the biologically induced allergens such as pollens are mainly composed of proteins as described above. Therefore, the inventors thought that the activity of the allergen may be extinguished by decomposing or denaturing the proteins contained in the pollen.

It has been known that proteins are generally decomposed by chemical denaturation with an acid or alkali, physical denaturation at high temperature, and biochemical denaturation with protein decomposing enzymes. However, it has not been reported that properties are converted into epitopes inherent to the proteins by denaturation of the protein, or the activity as the allergen is specifically inactivated. Moreover, an effective allergen inactivating method that can be understood by biochemical knowledge has not been reported yet. It is epoch-making that the inventors have elucidated that the allergen, in particular, the pollen allergen could be inactivated by denaturation of the protein.

According to the first allergen inactivating method of the invention, the allergen can be inactivated by maintaining the allergen under a condition in which any one selected from the group consisting of heat, an alkali, an acid and an enzyme exists.

The allergen inactivating methods using heat, an alkali, an acid and an enzyme will be described below.

[Case of Using Heat]

When the allergen is inactivated in the presence of heat according to the invention, the allergen may be heated, for example, at 80° C. or more for about 60 minutes, and at 65° C. or more for about 120 minutes. The allergen may be inactivated at a temperature not higher than 65° C. by prolonging the heating time.

In one aspect of the invention, the invention was achieved based on a unique and novel idea that heat may be used for inactivating the allergen. Accordingly, one of the crucial points of the invention is to inactivate the allergen using heat, not in the conditions such as the temperature and heating time. In other words, all the possible combinations of the conditions such as the temperature and heating time necessary for inactivating the allergen by heat fall within the scope of the invention.

The phrase “in the presence of heat” as used herein may be achieved by directly heating the allergen or an object containing the allergen using a heat source known in the art, per se, or by indirectly heating the allergen or an object containing the allergen via a heated medium.

When the allergen is inactivated according to the invention, the allergen may be heated in addition to treating with the protease and denaturing agent. The phrase “in the presence of heat” as used herein means to heat the allergen or an object containing the allergen using a heat source known per se, or to indirectly heat or worm the allergen or an object containing the allergen via a heated medium. It is desirable in home electric appliances such as an air conditioner that the allergen is decomposed at about 30° C. (or near the room temperature) considering the limitation of the construction of the apparatus. However, heat resistant enzymes such as Pfu is preferably used at higher temperatures (80 to 90° C.). While it is not impossible to maintain such high temperatures, the temperature is not preferable in home electric appliances for saving energies and for ensuring safety of the product. It is possible according to the embodiment of the invention to inactivate the allergen in within shorter time than usual by heating at least at 30° C., when the allergen is inactivated with the protease and a denaturing agent. However, the allergen may be inactivated with the protease and denaturing agent at a temperature of 30° C. or less, for example, about 20° C. or lower.

Similarly, the temperature for inactivating the allergen is also restricted in the home electric appliances such as a vacuum cleaner and bedclothes drier that may such allergen substances such as mites and viruses into the inner space. Since a protective wear is also provided with an apparatus for cleaning air at the site in the vicinity of the body surfaces, the temperature should be controlled considering the effect on the human body.

Examples of the denaturing agent used in the invention include surfactants and salts. Examples of the surfactant include anionic surfactants such as sodium dodecylsulfate (SDS), lithium dodecylsulfate, 3,5-didodecylsulfate, tris(hydroxymethyl)aminomethane dodecylsulfate, sodium cholate, N-lauroylsarcosine and sodium N-lauroylsarcosinate; cationic surfactant such as cetyldimthylethylammonium bromide and cetyltrimethylammonium bromide; amphoteric surfactants such as 3-[(3-cholamidepropyl)dimethylammonide]-2-hydroxy-1-propane sulfonate and 3-[(3-cholamidepropyl)dimethylammonide)]-1-propane sulfonate; and nonionic surfactant such as polyoxyethylene nonylphenylether (APE). Examples of the salt include guanidine hydrochloride.

[Case of Using Alkali]

When the allergen is inactivated in the presence of an alkali according to the invention, allergen may be added in an aqueous 2.5 M NaOH solution, or in an aqueous solution of a basic substance similar to the aqueous 2.5 M NaOH solution. Any substances capable of generating hydroxide ions by dissolving in water may be used for obtaining the aqueous alkaline solution in the invention. For example, the basic substances include various salts such as sodium hydroxide and potassium hydroxide, and strongly basic anion exchange resins, although the substance is not restricted thereto. Efficient inactivation is possible by heating in addition to the alkaline condition. The heating temperature and heating time are adjusted when the allergen is treated with a combination of an alkali and heat. This may permit favorable inactivation to be attained even by using lower concentration of the basic substance than in the condition described above, or by using a more weakly alkaline substance as compared with the strongly basic substances described above.

Inactivation of the allergen was achieved in one aspect of the invention based on a unique and novel idea of using an alkali for deactivating the allergen. On of the crucial points of the invention is to inactivate the allergen using an alkali, per se, rather than to determine detailed conditions of the alkaline solution. In other words, all the possible combinations such as pH, concentrations of the basic substance, heating or not heating, heating temperature and heating time fall within the scope of the invention.

[Case of Using Acid]

Allergen may be added, for example, to a HCl solution in a concentration of about 2.5 M, or to an aqueous solution of an acidic substance capable of attaining the condition in the presence of HCl, for inactivating the allergen in the presence of an acid, although the condition is not restricted thereto. Any substances that generate hydrogen ions by dissolving in water may be used as the substance available for obtaining the aqueous solution of an acid. Examples of the substance include acids such as hydrochloric acid and sulfuric acid, and strongly acidic cation exchange resins, although the substance is not restricted thereto. Efficient inactivation is possible by heating in addition to an acidic condition. Favorable inactivation of the allergen is possible by using a lower concentration of the acidic substance or more weakly acidic substances than the strongly acidic substances by adjusting the heating temperature and heating time in treating the allergen in a combination of heat and an acid.

Inactivation of the allergen was achieved in one aspect of the invention based on a unique and novel idea of using an acid for inactivating the allergen. One of the crucial points of the invention is to inactivate the allergen using an acid, per se, rather than to determine detailed conditions of the acidic solution. In other words, all the possible combinations such as pH, concentrations of the acidic substance, heating or not heating, and heating temperature fall within the scope of the invention.

[Case of Using Enzyme]

Examples of the enzyme include a protease. The term “protease” as used herein collectively denotes enzymes having properties for decomposing proteins and peptides. The enzymes available in the invention may be any one of acidic, neutral and basic proteases known in the art, per se. For example, they may be serine proteases such as trypsin, cysteine proteases such as papain, calpain and cathepsin B and cathepsin L, aspartic acid proteases such as pepsin, renin and cathepsin D, and proteases such as metalloprotease. The conditions of the temperature and coenzymes may be appropriately selected by the researchers considering the optimum condition of the protease used, when the allergen is inactivated with the protease.

More efficient inactivation is possible by adding urea as a denaturing agent in addition to the protease used, when the allergen is inactivated with the protease according to the invention.

The concentration of the protease used in the invention is, for example, about 1 unit per 0.45 μg of the allergen protein when pfu is used. The effect obtained is different depending on the treating temperature and time, and the presence of urea, when the protease is used. Accordingly, the concentration of the protease used is not restricted to that described above, and the protease may be effectively used under various conditions.

[Conditions]

The optimum concentrations of the protease and denaturing agent used are as follows when the allergen is inactivated with the protease and denaturing agent. For example, While the optimum condition for using the surfactant as the denaturing agent may be in the range of 0.1 to 2%, the concentration is not restricted thereto. The optimum concentration of protease is about 1 unit per 0.45 μg of the allergen protein when pfu is used as the protease. However, it has been described above that the decomposition rate of the allergen is largely improved depending on the presence of the denaturing agent. The amount of use may be reduced depending on the use and concentration of the denaturing agent. The effect obtained is also varied depending on the treating temperature and time when the protease is used. Accordingly, the concentration of the protease used is not restricted to that described above, and the protease may be effectively used in various conditions.

In one aspect of the invention, the invention was achieved based on a unique and novel idea that the protease and denaturing agent heat may be used together for inactivating the allergen. Accordingly, one of the crucial points of the invention is to inactivate the allergen using the protease and denaturing agent together, not in the detailed conditions such as the concentrations thereof, treating time, heating temperature and heating time. In other words, all the possible combinations of the conditions such as the kind and concentration of the enzyme related to inactivation of the allergen with the protease and denaturing agent, the concentration of the denaturing agent, and conditions such as heating or not heating, heating temperature and heating time fall within the scope of the invention.

When the allergen is inactivated with the protease and denaturing agent, the temperature and coenzymes may be changed considering the optimum condition of the protease and denaturing agent used.

2) Application to Air Conditioner

According to another aspect of the invention, the method described above may be used for an air conditioner. Accordingly, the invention provides an air conditioner comprising an allergen inactivating part for inactivating the allergen by maintaining the allergen under a condition in which any one selected from the group consisting of heat, an alkali, an acid and an enzyme exists. The invention also provides an air conditioner comprising an allergen inactivating part for inactivating the allergen by maintaining the allergen under a condition containing the protease and denaturing agent.

The allergen inactivating part applicable in the invention may be means capable of maintaining the allergen under a condition in which any one selected from the group consisting of heat, an alkali, an acid and an enzyme exists. Otherwise, the allergen inactivating part may be means capable of maintaining the allergen in the presence of the protease and denaturing agent. An arbitrary heating element known in the art, per se, may be used when the allergen is kept in the presence of heat. A commonly known chromatographic technique may be used, for example, for maintaining the allergen in the presence of an alkali, acid or enzyme. In the chromatographic method, a compound capable of supplying an alkali and acid, or an enzyme, is immobilized on a filter or particles to treat the allergen contained in the mobile phase.

Examples of the air conditioner using the filter according to the invention include air conditioners for use in houses as well as industrial air conditioners used in buildings and air conditioners for vehicles.

3) Other Applications of Air Cleaning Function

Furthermore, paying attention to the air cleaning function of the air conditioner described above, the method of the invention is also applicable to an air cleaner as a similar air treating apparatus, and to apparatus and facilities for cleaning air in a given space such as public dust collecting facilities provided with an air cleaning unit. In addition, the method of the invention is also applicable for cleaning exhaust air of home electric appliances such as a vacuum cleaner and bedclothes drier in which air collected from the inside space is blown out, and for cleaning sucked air for blowing air into a given space such as a mask for a human body, surgical and medical clean room facilities, biological terrorism countermeasure facilities and biological terrorism protecting clothes.

Examples of the filter applicable in the invention are shown below.

[Allergen Inactivating Filter by Heating]

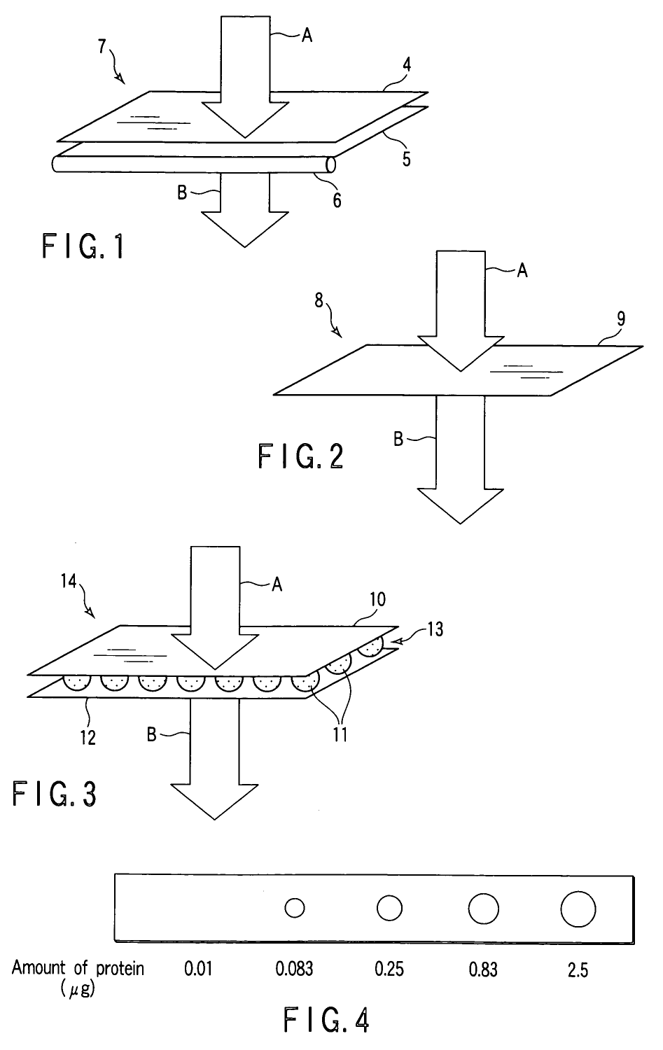

A first example of the filter according to the invention comprises, as shown in FIG. 1, a nonwoven fabric filter 4 for trapping the allergen, a stainless steel heating element 5 disposed at one surface of the nonwoven fabric filter 4, and a heater 6 for heating the stainless steel heating element 5.

Examples of the stainless steel heating element 5 include a stainless steel fiber mesh, although it is not restricted thereto. Since the allergen inactivating filter 7 is usually used by being integrated with the air conditioner, the power source of the heater 6 preferably uses the power source of the air conditioner. While the heater 6 is heated at a given temperature for a given time period when the air conditioner is OFF, the heating temperature is high to an extent not damaging the components of the air conditioner when the heating time is short, while the heating temperature is low when the heating time is long. However, the heating temperature and time may be appropriately determined depending on the materials of the components. The allergen protein is denatured by heating the heating element 5 with the heated 6 to extinguish the activity as the allergen.

[Allergen Inactivating Filter Retaining Acid or Alkali]

A second example of the filter according to the invention is characterized in that a strongly acidic or basic ion exchange resin is retained on a nonwoven fabric filter 9 as shown in FIG. 2. Such an allergen inactivating filter 8 can be manufactured by weaving an ion exchange resin (may be fibrous or spherical) into the usual filter.

i) Strongly Acidic Cation Exchange Resin

For example, strongly acidic cation exchange resins represented by a general formula —SO₃.H (R represents a polymer frame) can be used in the invention. Since this resin can be regenerated using an acid, it should be activated using the acid before use.

ii) Strongly Basic Anion Exchange Resin

For example, strongly basic cation exchange resins represented by a general formula below can be used in the invention. Since this resin can be regenerated using a base, it should be activated using the base material before use. 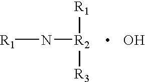
Provided that R₁, R₂ and R₃ Denote Polymer Main Frames.

The allergen protein maintained in the presence of an acid or alkali is denatured by their pH conditions to extinguish the activity as the allergen.

[Allergen Inactivating Filter Immobilizing Enzyme]

In a third example, the filter according to the invention/comprises, as shown in FIG. 3, a first supporting member 10 having meshes smaller than the particle diameter of a water absorbing polymer, a second supporting member 12 disposed at one side of the first supporting member 10 and having meshes smaller than the particle diameter of a water absorbing polymer, and a water absorbing polymer layer 13 disposed between the first supporting member 10 and the second supporting member 12.

The carriers of the enzyme available include water absorbing polymers such as an acrylic acid-vinyl alcohol copolymer and an acrylic acid-sodium acrylate polymer. The water absorbing ability of the carrier is preferably at least about 600 g per 1 g of the dry weight of the water absorbing polymer.

While the first and second carriers are made of resins, the material is not restricted thereto. The water absorbing polymer 11 preferably contains a predetermined quantity of the protease. The allergen inactivating filter 14 immobilizing the enzyme is manufactured, for example, by coating the filter with an enzyme solution after preparing a filter in which the carrier is woven, or by weaving the carrier coated with the enzyme solution into the filter. Contamination by fungi may be reduced by keeping the relative humidity (or moisture activity) at 70% or less. More advanced effects may be expected by adding a surfactant (at a concentration of, for example, about 0.1%).

Allergy is also caused by direct contact of the allergen on the skin. However, most cases of allergy is caused by inhalation of floating allergens in air. Accordingly, allergic symptoms may be alleviated by excluding the allergen in air. Consequently, the allergens floating in air may be inactivated using the apparatus according to the invention to enable remarkable improvement of house environment to be expected.

[Allergen Inactivating Filter Immobilizing Enzyme and Denaturing Agent]

Examples of the filter according to the invention are the same as the filter shown in FIG. 1. Accordingly, the contents different from the contents described in the “allergen inactivating filter immobilizing the enzyme” will be described in this section. The water abosrbing polymer preferably contains predetermined quantities of the protease and denaturing agent. The allergen inactivating filter immobilizing the enzyme and denaturing agent may be prepared either by coating the filter having a carrier woven therein with a solution containing the enzyme and denaturing agent, or weaving the carrier coated with the solution containing the enzyme and denaturing agent into the filter.

(2) In a Second Allergen Inactivating Method of the Invention, the Allergen is Maintained Under a Condition in which Any One Selected from the Group Consisting of an Alkali, an Acid and an Enzyme, and Heat Exist.

The concept of the alkali, acid, enzyme and heat, and applications to an air conditioner are the same as described in the method (1).

According to the second allergen inactivating method of the invention, the allergen may be inactivated by maintaining the allergen under a condition in which any one selected from the group consisting of an alkali, an acid and an enzyme, and heat exist.

(3) The Allergen Inactivating Filter According to the Invention Comprises a Filter Main Body, and Activating Means of Heat, an Acid, an Alkali and an Enzyme Having an Allergen Inactivating Function.

While the enzymes are not particularly restricted in the invention so long as it is possible to denature or decompose the proteins constituting the allergen, examples thereof include a protease and peptidase. The protease is an enzyme that hydrolyzes peptide bonds to degrade the protein into peptones. The peptidase functions to hydrolyze peptide bonds of amino or carboxyl terminals of a peptide chain. While acidic, neutral and basic enzymes with an activity of 1,000,000 units (the unit of an enzyme that hydrolyzes 1 μmol of protein per minute) are available, enzymes with an activity more than 1,000,000 units may be used without any problem.

The materials of the filter main body available include natural fibers such as cotton and wool; regenerated fibers such as rayon and cellulose acetate fibers; nonwoven or woven fabrics of synthetic fibers such as polyethylene, polyethylene terephthalate and polyamide fibers; glass fiber mats; metal fiber mats; synthetic resins such as acrylic, acrylamide and polyvinyl alcohol resins; and water absorbing and/or moisture absorbing materials as natural and regenerated materials of sodium alginate, mannan and agar. The enzyme is immobilized directly or via a carrier on the filter main body comprising these materials.

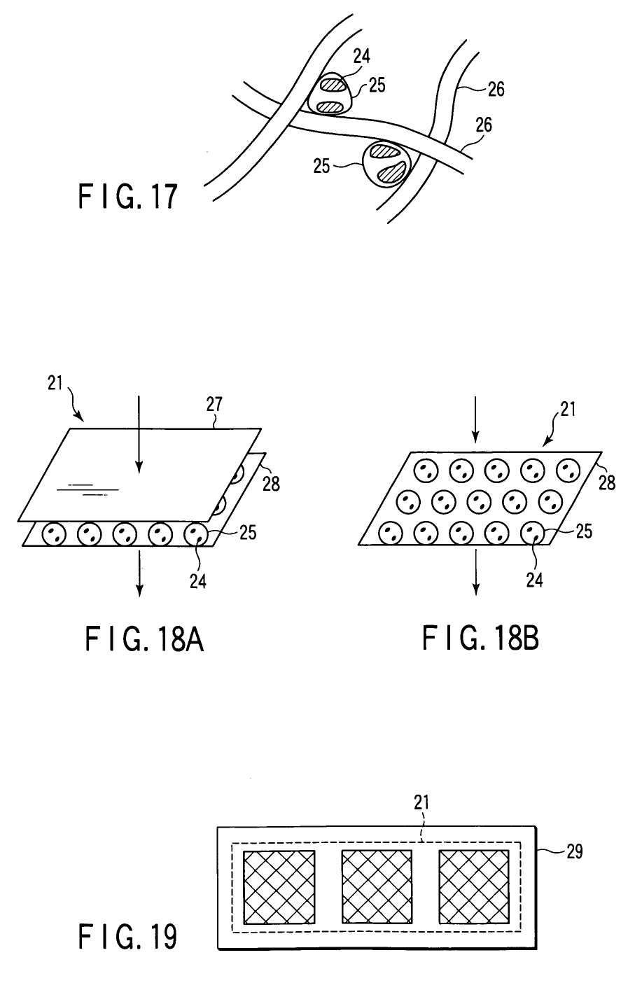

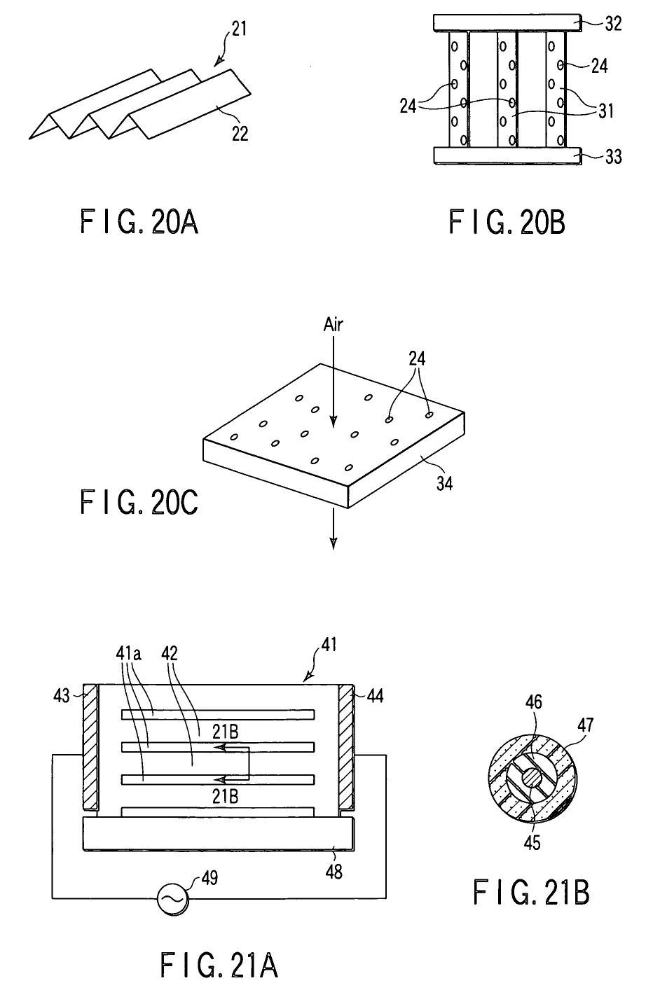

While the shape of the filter main body (filter) is not particularly restricted, examples of the shape are as follows:
1) a flat filter comprising an enzyme having an allergen inactivating function on the flat filter main body made of, for example, a nonwoven fabric (see FIG. 16);
2) a flat filter comprising a carrier retaining the enzyme immobilized on the nonwoven fabric using a binder (see FIG. 17);
3) a flat filter comprising the enzyme-immobilizing carrier sandwiched between two base materials (fiber sheets; see FIG. 18A), or a flat filter as an open sandwich on the base material (see FIG. 18B);
4) a pleated filter having folds on the filter main body (see FIG. 20A), which may increase the surface area of the filter main body;
5) a rod shape filter comprising rod-shaped members comprising bundles of fibers immobilizing the enzyme, the rod-like members being linked at both sides thereof (see FIG. 20B; the cross section of the rod-shaped member is not particularly restricted, and includes a triangle, rectangle, circle, ellipsoid and hollow shape);
6) a sponge-like filter immobilizing the enzyme on the surface of a porous material such as polyurethane (see FIG. 20C); and
7) a louver-like filter having a louver function at the opening of thin louver plates of, for example, an air conditioner (not shown).

The filter main body in the invention preferably comprises the enzyme having an allergen inactivating function while it is endowed with at least one function at least one of deodorizing function, dust removing function, bactericidal function, aroma adding function and negative ion adding function.

Since the enzyme usually has no activity under an absolutely dry state, replenishment of water such as moisture in air or supplied liquid water is necessary. Accordingly, a pool of water is disposed above or below the filter, or at the position near the filter so as not to block the ventilation passageway, in order to permit the water pool to directly communicate the filter. Otherwise, the filter is connected to a water permeable material member of a material having a capillary action to enable water required for activating the enzyme in the filter to be always replenished from the water pool.

Examples of the material having a capillary function include Japanese paper, absorbent cotton and a mat or fabric of wool. A material having the same water absorbing property as the material used for the filter may be used alone, or by mixing with Japanese paper, absorbent cotton and a mat or fabric of wool. The water absorbing material is favorably used when water is replenished as liquid water. On the other hand, a moisture absorbing material is favorably used when water is replenished from moisture in air. It is needless to say that materials having the water absorbing and moisture absorbing properties together may be also favorably used.

The vessel constituting the water pool is preferably made of a material impermeable to water in order to prevent the loss of water. Examples of the material include metallic materials such as aluminum and iron, and plastic materials such as polyethylene and polypropylene. While the shape of the vessel is not particularly restricted, it is preferable that the vessel is a slender cylinder along the edge of the filter with a slit or small holes provided in the longitudinal direction for replenishment of water to the filter. While water may be directly filled in the vessel, a water absorbing material is desirably filled in the vessel in order to prevent water from being leaked. The same material as used for the filter may be used as the water absorbing material for replenishing water to the vessel.

Replenishing water available includes city water and distilled water, as well as condensed water generated in the heat exchanger when cooling and absorbed water obtained by taking advantage of high relative humidity of cooled air. No special treatment of water is necessary in any cases. However, adding a surfactant (about 0.1%) in replenishing water permits wettability of the enzyme with the allergen to be increased to enable an additional effect to be expected.

The enzyme cannot exhibit a sufficient activity when the temperature of air is low, particularly when the air conditioner is in a continuous cooling operation in the summertime. It is preferable in such a case to provide a heater on the allergen inactivating filter of the invention in order to inactivate the allergen by heating with the heater. For example, the filter is heated by turning the heater ON when the air conditioner is OFF by providing the filter in the air conditioner, in order to denature the allergen protein by heating and to extinguish the activity as the allergen to allow allergen-free air to pass through the filter. Otherwise, the temperature of the enzyme may be increased by temporarily switching the operation of the air conditioner from cooling to warming operation, in place of providing the heater.

The temperature of air is also low immediately after starting warming operation in the wintertime. Particularly, it takes a long time before air around the filter is warned after the start of warming operation, when the filter is provided at the air suction side of the air conditioner. However, providing the heater as described above permits air around the filter to be warmed immediately after the start of operation to enable the allergen inactivating activity of the enzyme to be exhibited at an early stage of operation.

According to the allergen inactivating filter described in (3) above, the amount of the allergen can be reduced by providing inactivation means having an allergen inactivating function on the filter main body. An air treating apparatus capable of reducing the amount of allergen is obtained by providing the filter in the air conditioner or the like. Furthermore, the air treating apparatus capable of reducing the amount of allergen is obtained by switching the operation mode can be obtained by providing an allergen removing operation mode. Since the air treating apparatus is not needed to use an anti-allergen agent, adverse effect on the living body may be eliminated.

(4) The air treating apparatus according to the invention comprises the allergen inactivating filter in (1) or (2) above.

An example of the air treating apparatus is any one of the air conditioner for houses, offices or vehicles, air cleaner, dehumidifier and dryer. Such air treating apparatus preferably comprises an allergen removing operation mode. The allergen removing operation mode is provided with operation mode selection means to enable allergen removing operation when necessary. Therefore, the amount of the allergen can be reduced by switching the operation mode.

Since the air treatment apparatus described above comprises the allergen inactivating filter, the amount of the allergen can be reduced.

The virus inactivating agent and virus inactivating method according to the invention, the filter provided with the inactivating agent, and the air conditioner, air cleaner and air cleaning unit for facilities which comprise the filter will be described below.

(5) The virus inactivating agent according to the invention contains at least one selected from the group consisting of a protein denaturing agent and a protein decomposing enzyme.

The virus inactivating agent of the invention may contain both the protein denaturing agent and protein decomposing enzyme. The protein denaturing agent and the enzyme are preferably urea and a protease, respectively, and the enzyme is preferably pfu protease S.

The virus is preferably a virus having envelopes or a virus without the envelopes.

The inactivating agent has a bactericidal action against microorganisms, especially against bacteria, as well as against eukaryotic cells such as fungi.

The virus inactivating agent of the invention is able to inactivate the virus in a liquid phase. However, minute liquid phases are formed on the surface of the fiber or within the carrier particles by using a moisture absorbing material for the carrier to enable the inactivating agent to be activated.

Viruses are infectious substances usually having basic structures comprising proteins and nucleic acids. The invention is based on the elucidation by the inventors that the virus may be inactivated by using the protein denaturing agent or protein decomposing enzyme as active ingredients, the agent is harmless to human bodies, the effect of the invention can sustain for a long period of time, and inactivation of the virus is possible with an economically excellent method. The virus inactivating agent of the invention is readily integrated into an apparatus such as the air conditioner, and is excellent for practical applications.

The protein denaturing agent used in the invention denatures the virus protein to inactivate the virus by protein denaturation. Examples of the protein denaturing agent available include urea, guanidine hydrochloride, and surfactants such as sodium dodecylsulfate (SDS).

The protein decomposing enzyme collectively denotes enzymes capable of decomposing proteins and peptides. The protein decomposing enzyme used in the invention inactivates the virus by decomposition of virus proteins, and proteases are particularly used. The protease may be any one of acidic, neutral and basic proteases known in the art. For example, they may be serine proteases such as trypsin, cysteine proteases such as papain, calpain and cathepsin B and cathepsin L, aspartic acid proteases such as pepsin, renin and cathepsin D, and proteases such as metalloproteases. However, pfu protease S manufactured by Takara Bio Inc. is the most preferable protease. This protease has high heat resistance and maintains high resistance in the existence of the denaturing agent such as urea and SDS.

Conditions such as the temperature and coenzymes may be appropriately selected by the researchers for inactivating the virus with the protease considering the optimum condition of the protease used.

Inactivation of the virus in the invention means extinguishing the infectious ability of the virus. The infectious ability of the virus may be extinguished by denaturing or decomposing the virus protein with active components such as the denaturing agent and enzyme. The bactericidal and fungicidal action means to destroy the bacteria or fungi, or to inhibit growth thereof.

The reactions using the enzyme and denaturing agent are preferably carried out in a liquid phase. Accordingly, it is also preferable to use the virus inactivating agent of the invention in a solution or similar liquid phase.

In one aspect of the invention, the invention was achieved based on a unique and novel idea that the protein decomposing enzyme and protein denaturing agent are used together for inactivating the allergen. Accordingly, one of the crucial points of the aspect of the invention is to inactivate the allergen by taking advantage of using the protein decomposing enzyme and protein denaturing agent together, not in the detailed conditions such as the concentration and treatment time thereof. In other words, all the possible combinations of the conditions such as the kind and concentration of the protein decomposing enzyme, and the kind and concentration of the protein denaturing agent fall within the scope of the invention.

Some viruses have bacteriolytic enzymes having similar effect to the protein decomposing enzyme used in the invention. Since such viruses are resistant to the enzyme, it is difficult to inactivate the virus with the protein decomposing enzyme of the invention. However, it may be an advantage that the decomposition enzymes readily act on the enzyme resistant viruses by denaturing the virus protein with the protein denaturing agent. The concentration of the decomposi-tion enzyme may be reduced by using the protein denaturing agent and protein decomposing enzyme together.

According to the aspect of the invention, more efficient inactivation of the virus becomes possible as will be shown in the examples hereinafter, by using the protein denaturing agent and protein decomposing enzyme together.

Some viruses have a membrane structure surrounding the outside of the protein structure, which is called as an envelope. The envelop comprises lipid bilayers as in cell membranes, and virus specific proteins are present thereon. The object of the invention is both the viruses having the envelope and not having the envelope, and provides an inactivation agent and inactivating method for a wide range of viruses.

(6) In the virus inactivating method of the invention, the viruses are made to contact a solution containing the virus inactivating agent.

The virus inactivating method using the protein denaturing agent and protein decomposing enzyme will be described hereinafter.

Urea may be used for inactivating the virus using the protein denaturing agent of the invention preferably in a concentration of about 9 M.

Protease may be also used for inactivating the virus using the protein denaturing agent of the invention preferably in a final concentration of, for example, 2% when pfu protease S (manufactured by Takara Bio Inc.) is used. The effect obtained varies depending on the conditions such as the treating temperature and time when the protease is used. Accordingly, the concentration of the protease used is not restricted to the range above, and may be effectively used under various conditions.

The virus can be efficiently inactivated by using the protein denaturing agent and protein decomposing enzyme together, when the virus is inactivated according to the invention. For example, while the final concentration is about 2% when pfu protease S (manufactured by Takara Bio Inc.) is used alone, a sufficient inactivation effect may be obtained at the concentration of about 2% when urea is used together in a 9 M concentration.

(7) The virus inactivating filter according to the invention comprises a filter for trapping the virus and the virus inactivating agent adhered on the filter.

The filter for trapping the virus is made of, for example, a nonwoven fabric. The virus inactivating agent comprises at least one active component selected from the group consisting of the protein denaturing agent and protein decomposing enzyme. The virus is trapped and inactivated by adhering the virus inactivating agent on the filter. The virus inactivating agent can be immobilized on the carrier such as a filter or particles by the method to be described hereinafter for adhering the virus inactivating agent on the filter.

Examples of the materials of the filter available include fibers of polyethylene (PE), polypropylene (PP), polyethylene terephthalate (PET) and polyamide (PA). Examples of the materials of the carrier available include water absorbing polymers such as an acrylic acid-vinyl alcohol copolymer and an acrylic acid-sodium acrylate polymer.

A water repelling fiber may be combined when the filter is made of moisture absorbing fibers. The filter is made to be possible to maintain its shape by combining the water repelling polymer, and the filter is prevented from being collapsed even when it absorbs a large quantity of water. While various shapes are possible for forming the filter, a pleated shape is an example of the shape of the filter. The shape retaining effect by the water repelling fiber is particularly effective in the filter having the pleated shape.

The shape of the filter may be maintained without being collapsed by combining with the water repelling fiber even when a large amount of moisture is incorporated into the filter by immersion or spraying for adhering the inactivating agent on the filter. Accordingly, it is important to combine the water repelling fiber with the moisture absorbing fiber before the inactivating agent is adhered on the filter.

Maintaining the shape of the filter permit the amount of the inactivating agent to be uniformly immobilized per unit area of the filter.

While the filter provided in the air conditioner suffers an external force by the wind pressure as will be described below, the strength of the filter is enhanced by adding the water repelling fiber by preventing the filter from being deformed.

(8) The air conditioning unit according to the invention comprises a suction port for sucking air, a heat exchanger for cooling or heating by heat exchange between the air sucked from the suction port and a refrigerant, a blowing port for flowing the air heat-exchanged by the heat exchanger back into the room, ventilation means for blowing out the air sucked from the suction port and heat-exchanged by the heat exchanger into the room, a virus inactivating filter disposed in an inner space through which air flows and immobilizing the virus inactivating agent, and inactivating agent activating means for maintaining the inner space in an atmosphere in which the virus inactivating agent is activated.

While examples of the air conditioning unit include an indoor air conditioning unit, the unit is not restricted thereto. The indoor air conditioning unit is preferably provided with opening/closing means for maintaining the inner space in a semi-hermetic or hermetic state by closing a part or all part of an opening communicating with the inner space. The inner space of the air conditioning unit is maintained in the hermetic state, and air adjusted in an atmosphere for activating the virus inactivating agent in the hermetic state is preferably agitated by operating the indoor ventilation means. Furthermore, the indoor air conditioning unit is preferably operated for preventing the inactivating agent from being deteriorated by removing moisture from the inactivating agent carrier, after maintaining the inner space high temperature/high humidity with the inactivating agent activating means.

It is further preferable that the indoor air conditioning unit is operated for trapping the virus before activating the virus inactivating agent on the inactivating agent carrier, by sucking indoor air into the inner space and allowing the air to flow through the inactivating agent carrier.

While examples of the heat exchanger include an indoor heat exchanger, it is not restricted thereto. For activating the inactivating agent, condensed water generated by cooling operation of the indoor heat exchanger is preferably vaporized by heating operation of the indoor heat exchanger after the cooling operation. Otherwise, condensed water generated by cooling operation of the indoor heat exchanger and pooled in a drain receiver is preferably vaporized by heating with heating means.

Using the indoor heat exchanger in the indoor air conditioning unit permits the amount of the virus in the room to be reduced.

(9) The air conditioner according to the invention comprises the air conditioning unit, a second air conditioning unit having a compressor for compressing the refrigerant and a heat exchanger for heat exchange between the refrigerant and air; and a refrigerant piping for connecting between the two air conditioning units and for allowing the refrigerant to circulate between the two air conditioning units.

The air conditioner may be employed for inactivating the virus in the invention. According to one aspect, the invention provides the virus inactivating filter. The invention also provides the air conditioner provided with the virus inactivating filter in the indoor air conditioning unit.

The air conditioner provided by the invention comprises the virus inactivating filter. The virus inactivating filter is provided in the indoor air conditioning unit, air sucked by operating the air conditioner is made to flow through the filter. Such construction permits the virus in the room to be trapped and inactivated by operating the air conditioner.

### EXAMPLES 1 TO 12

Examples of the invention will be described in detail with reference to the drawings. The elements having the same functions are given the same reference numerals in the drawings to be described below, and repetition of descriptions are omitted.

The allergen detection method, materials and allergen will be described at first.

Method and Material

(1) Detection of Allergen

It is desirable to employ a detection system similar to human and animals' own allergic reactions for detecting the allergen, if any. Since the allergic reaction is an antibody-antigen reaction, the inventors employed measuring methods using antigens.

(2) Allergen

Mite extracts were used as one of the allergens in the experiments in the examples below. The mite extracts used were mite extract-Df (a supernatant of ground mite bodies in a phosphate buffer was freeze-dried; manufactured by Takara Bio Inc.), and 10 mg of the extract was dissolved in 2.5 ml of PBS solution according to the prescription. The solution obtained is named as a mite preparation, which was used in the experiment by diluting into an appropriate concentration with the PBS solution.

### Example 1

### Measurement of Allergen 1

Mite allergens in the mite extract were measured by Western blotting (usually named as a dot blotting method) in this example. The quantities of the mite extract used were 0.01, 0.083, 0.25. 0.83 and 2.5 μg as converted into the quantity of proteins.

A whole serum of rabbit (manufactured by Cosmo Bio Inc.; 2500 times dilution for use) was used as an antigens against the mite extract. An antigen against rabbit Ig modified with horseradish peroxidase (manufactured by Amersham Biosciences Co.) was used as an antigen against the whole serum of rabbit, and was diluted 1000 times before use.

ECL+Plus (manufactured by Amersham Biosciences Co.) as a Western blotting detection system was used for detecting the allergens, and was used according to the prescription manual of the manufacturer. The other operation procedures were in accordance with usual methods.

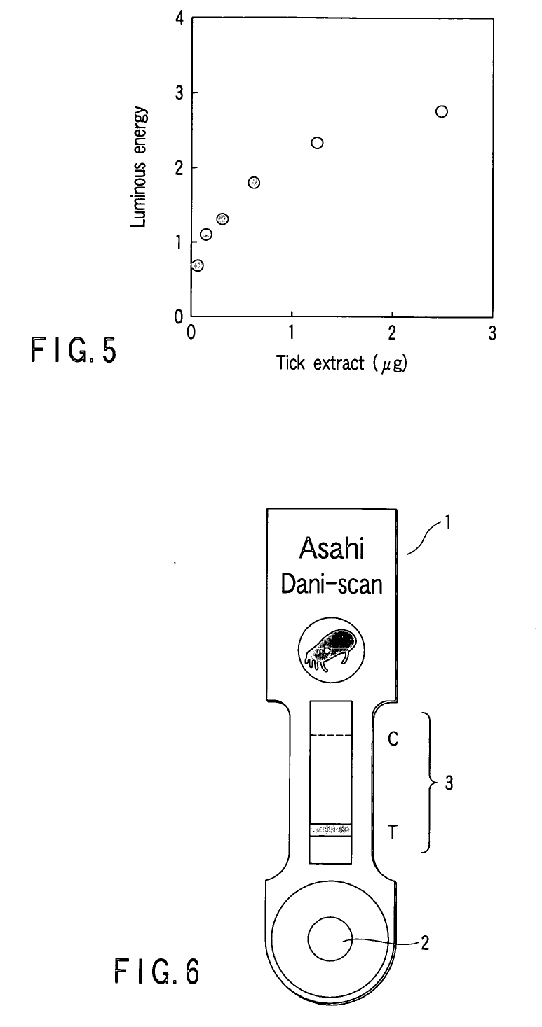

A calibration curve related to the amount of mite allergen in the mite extract was obtained from mite extract dependent luminous energies. FIG. 4 is a schematic diagram illustrating the result of the Western blotting. FIG. 4 shows that the luminous energy obtained was increased depending on the concentration. Accordingly, it was proved that the amount of the proteins in the mite extract reflects the amount of the mite allergen. FIG. 5 shows a calibration curve of numerical expressions of the luminous energy obtained by the Western blotting.

### Example 2

### Measurement of Allergen 2

The relative amount of the mite allergen in the sample was measured by mite scanning in the examples below including this example. The calibration curve for the mite allergen in the mite extract was prepared at first for confirming availability of mite scanning for quantitative analysis.

DaniScan (registered trademark; manufactured by Asahi Food and Healthcare Co.) was used as the mite allergen measuring system for detecting the allergen in a solution prepared by diluting the mite preparation with the PBS solution.

DaniScan is a kit for simply detecting the mite allergen using antigen-antibody reactions. Test samples are added to a sample addition part 2 of a dust collector 1 provided in DaniScan shown in FIG. 6. A band 3 reflecting the amount of the mite allergen in the test sample is obtained is obtained by adding a developer into the sample addition part. The relative amount of the allergen in the test sample can be detected from the band. The bands of DaniScan are expressed by “bold solid line”, “middle solid line”, “fine solid line”, “broken line” and “no line” for the convenience of recognition, and the luminous intensities are schematically illustrated in four ranks in the order of “bold solid line”>“middle solid line” >“fine solid line”>“broken line”>“no line”.

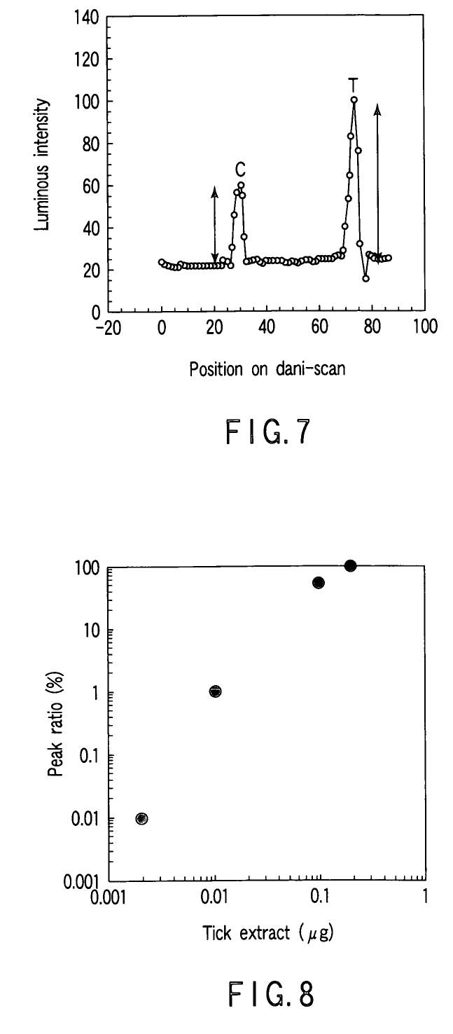

The quantity of the mite allergen contained in the sample was evaluated by calculating peak ratios according to the following equation from the luminous intensity peak value (T) derived from the mite antibody is bonded to the mite antigen and the luminous intensity peak value (C) derived from the mite antibody not bonded to the mite antigen in the mite extract. FIG. 7 is a graph showing the relation between the location of the band on the mite scanning and light emission intensity. The peak values (T) and (C) correspond to {C} and {T}, respectively, in FIG. 7.

The equation for determining the peak ratio from the luminous intensity peak (C) and luminous intensity peak (T):
Peak ratio (%) [T/(T+C)]×100
The amount of the mite allergen in the sample is small when C>T, while the mite allergen is contained in a large amount in the sample when T>C.

FIG. 6 is a schematic view showing the mite allergen positive band (when the allergen is contained in a large quantity). The band at position C is derived from the mite antibody not bonded to the mite antigen, while the band at position T is derived from the mite antibody bonded to the mite antigen. FIG. 8 shows the calibration curve related to the mite allergen in the mite extract.

### Example 3

### Inactivation of Allergen by Heat

The following experiments were carried out in order to prove the mite allergen inactivating action by heat treatment.

The mite preparation was diluted 10 times with the PBS solution. The 10 times dilution solution obtained was dispensed into seven micro-tunes, one of which was used as a reference with a heating time of zero minute. The remaining six tubes were heated at 80° C. on a heat block, which were maintained for 10, 20, 30, 40, 50 and 60 minutes, respectively. The samples were cooled on ice after heating. Sampled solutions (5 μL each) from the samples were added on DaniScan to detect the allergen. Since 5 μL of each sample added contains 2 μg of the mite extract as converted into the proteins, 2 μg of the mite extract is added to one DaniScan.

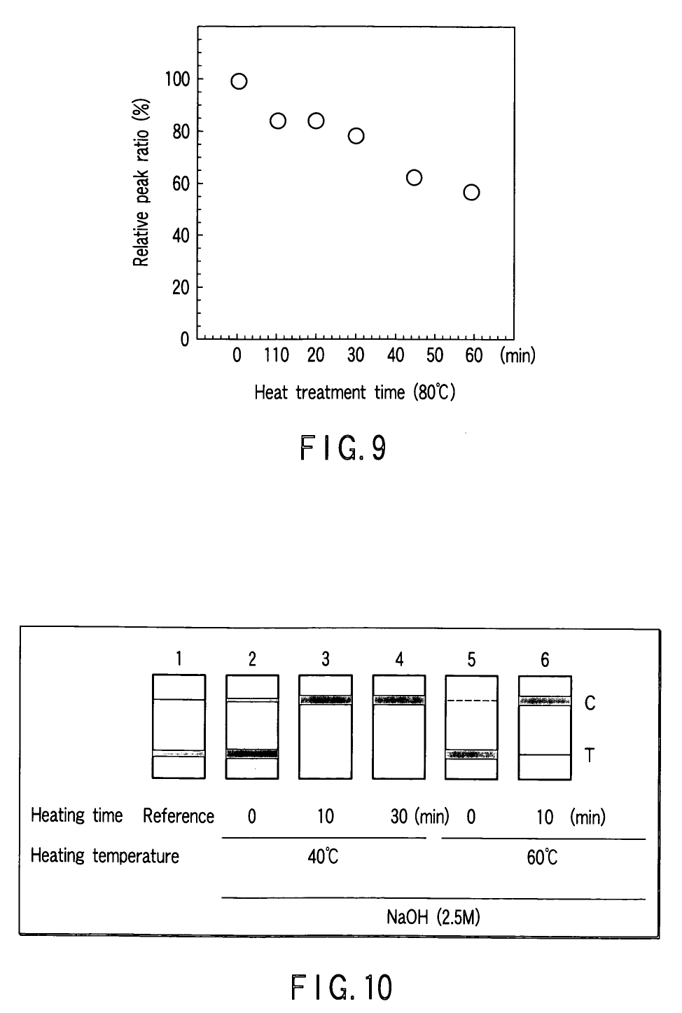

FIG. 9 is a graph showing the relation between the heat treatment time and the amount of the mite allergen in the sample. The relative peak ratio of the treated group when the peak ratio of the reference group is defined as 100 is shown in the vertical axis, and the heat treatment time is shown in the horizontal axis in FIG. 9.

FIG. 9 shows that the allergen is inactivated by the heat treatment. In particular, most of the allergen is inactivated by the heat treatment for 1 hour.

### Example 4

### Inactivation with Alkali

The following experiments were carried out to prove the mite allergen inactivation action by alkali treatment.

The mite preparation (50 μL) and 50 μL of a 5 M sodium hydroxide solution were mixed in each of five micro-tubes on ice. The final concentration of sodium hydroxide in each mixed solution was 2.5 M. Each micro-rube was maintained at 40° C. and 60° C. for 0, 10 and 30 minutes. After completing the reaction, the solution was neutralized by adding 50 μL of 5 M hydrochloric acid on ice. The reaction solution obtained (1 μL each; 1.33 g mite extract/DaniScan as converted into the protein) obtained was added to DaniScan to detect the mite allergen.

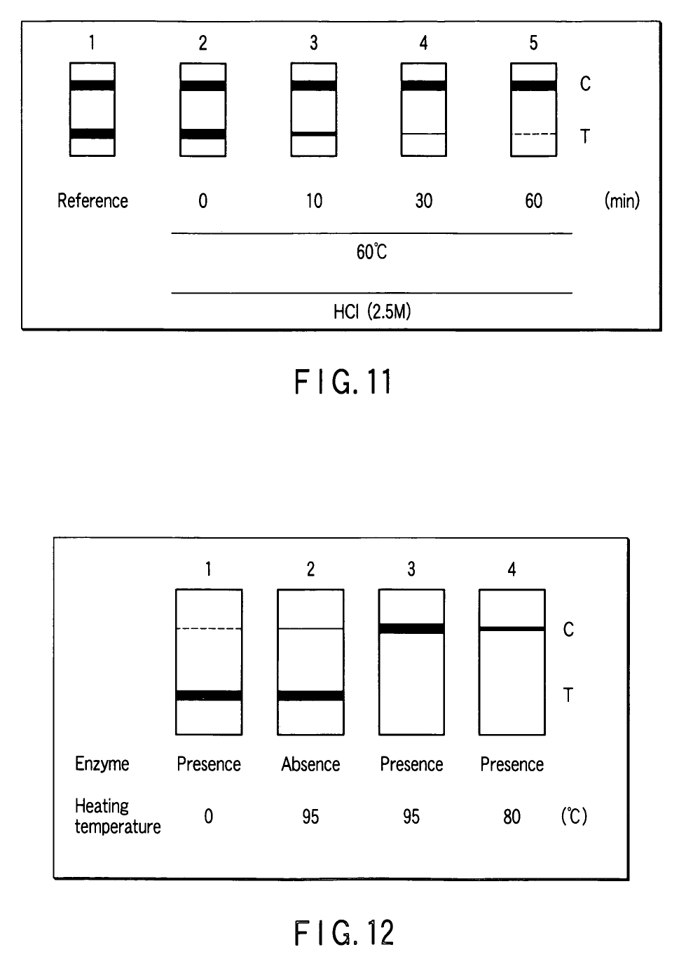

The results are shown in FIG. 10, which schematically illustrates the band observed in DaniScan after the reaction. The reference numeral 1 in FIG. 10 shows the result of the non-treated sample (or the result obtained by adding only 1.33 μg of the mite extract per one DaniScan), and is used as a reference sample. The reference numerals 2 to 4 in FIG. 10 show the results of the alkali treatments of the mite extract for 0, 10 and 30 minutes at 40° C. The reference numerals 5 and 6 in FIG. 10 show the results of the alkali treatments of the mite extract for 0 and 10 minutes at 60° C. FIG. 10 shows that the allergen is inactivated by treating at 40° C. and 60° C. for 10 minutes.

### Example 5

### Inactivation with Acid

The following experiments were carried out for proving the mite allergen inactivation action by treating with an acid.

The mite preparation (50 μL) and 50 μL of a 5 M hydrochloric acid solution were mixed in each of four micro-tubes on ice. The final concentration of hydrochloric acid in each mixed solution was 2.5 M. Each micro-rube was maintained at 60° C. for 0, 10, 30 and 60 minutes. After completing the reaction, the solution was neutralized by adding 50 μL of 5 M sodium hydroxide on ice. The reaction solution obtained (1 μL each; 1.33 g mite extract/DaniScan as converted into the protein) was added to DaniScan to detect the mite allergen.

FIG. 11 shows the results, which schematically illustrates the bands observed in DaniScan after the reaction. The reference numeral 1 in FIG. 11 shows the result of the non-treated sample (or the result obtained by adding only 1.33 μg of the mite extract per one DaniScan as converted into the protein), and is used as a reference sample. The reference numerals 2 to 5 in FIG. 11 show the results of the acid treatments of the mite extract for 0, 10, 30 and 60 minutes at 60° C. FIG. 11 shows that most of the allergen is inactivated by treating with the acid at 60° C. for 60 minutes.

### Example 6

### Inactivation with Protease 1

The following experiments were carried out for proving the allergen inactivating action with the protease.

Added in each of three micro-tubes on ice were 90 μL of the mite preparation and 8 μL (0.8 unit) of pfu protease S (abbreviated as pfu hereinafter, manufactured by Takara Bio Inc.). One of the three tubes was kept at 0° C. for 10 minutes, and another tube was heated at 95° C. for 10 minutes, and the rest was heated at 80° C. for 10 minutes. The sample with no addition of the enzyme was heated at 95° C. for 10 minutes. All the micro-tubes after the treatment were returned on ice, and a phenylmethylsulfonyl fluoride (abbreviated as PMSF hereinafter) solution as a protease inhibitor was added to each tube (the final concentration of PMSF is 4 mM).

A 5 μL each of solution extracted from each sample in which PMSF is added to detect the mite allergen by adding into DaniScan. 18 μg of the mite extract is added to each DaniScan as converted into the protein.

The results are shown in FIG. 12. The reference numeral 1 in FIG. 12 denotes the sample after a reaction at 0° C. for 10 minutes by adding the enzyme, the reference numeral 2 in FIG. 12 denotes a reference sample maintained at 95° C. for 10 minutes with no addition of the enzyme, the reference numeral 3 denotes the sample treated at 95° C. for 10 minutes by adding the enzyme, and the reference numeral 4 denotes the sample treated at 80° C. for 10 minutes by adding the enzyme. FIG. 11 shows that the mite allergen is inactivated by treating with the protease for 10 minutes at the optimum temperature described above.

### Example 7

### Inactivation with Protease 2

The following experiment were carried out for proving the mite allergen inactivating action by using the protease and urea together.

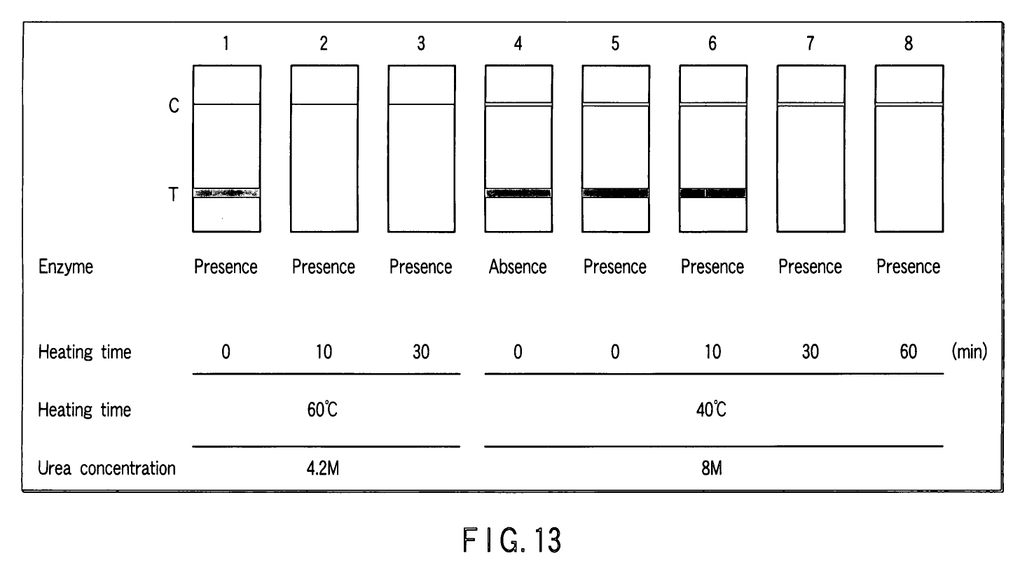

Added in each of three micro-tubes on ice were 20 μL of the mite preparation and 3.2 μL (0.32 unit) of pfu, and 16.8 μL of 10 M urea prepared using the PBS solution was further added to each sample solution. The final concentration of urea was 4.2 M. These sample solutions were heated at 60° C. for 0, 10 and 30 minutes. The reaction was stopped by returning each sample solution on ice after heating. The allergen was detected by sampling 1 μL each of the sample solution using DaniScan. The mite extract added to each DaniScan was 2 μg as converted into the amount of the protein. The results are shown in reference numerals 1 to 3 in FIG. 13. The reference numerals 1, 2 and 3 in FIG. 13 are the results of the samples heated at 60° C. for 0, 10 and 30 minutes, respectively.

The following experiments were also carried out by the similar manner. Added to each of the four micro-tubes were 12 μL of the mite preparation and 8 μL (0.8 unit) of pfu, and 80 μL of 10 M urea was further added to each tube. The final concentration of urea was 8 M. These tubes were then heated at 40° C. for 0, 10, 30 and 60 minutes. Sampled from each tube was 1 μL of the sample solution to measure the quantity of the allergen with DaniScan. The quantity of the mite extract added to each DaniScan was 0.48 μg as converted into the protein. The results are shown in the reference numerals 5 to 8 in FIG. 13. The reference numerals 5, 6 and 7 in FIG. 13 denote the results of the samples heated at 40° C. for 0, 10, 30 and 60 minutes, respectively.

A sample with no addition of the enzyme and no heat treatment was prepared and tested as a reference sample. Added in one micro-tube on ice was 12 μL of the mite preparation, 80 μL of 10 M urea, and 8 μL of the PBS solution. The final concentration of urea was 8 M. This micro-tube was not heated. Sampled from this tube was 1 μL of the sample solution to detect the allergen using DaniScan. The quantity of the mite extract added in one DaniScan was 0.48 g as converted into the amount of the protein. The results are shown by the reference numeral 4 in FIG. 13.

The results in FIG. 13 show that the allergen was inactivated 10 minutes after heating at 60° C. in the presence of 4.2 M urea. The allergen was inactivated 30 minutes after heating at 40° C. in the presence of 8 M urea.

### Example 8

### Inactivation with Protease 3

The following experiments were carried out for proving the mite allergen inactivation action by using papain as another example.

A 8 M urea-papain solution was prepared by mixing 400 μL of 10 M urea solution with 0.2 g/mL of papain (manufactured by Nagase Chemtechs Co., purified edible papain) and 100 μL of PBS solution.

Added in each of eight micro-tubes were 20 μL of the mite preparation and 80 μL of 8 M urea-papain solution. The final concentration of urea was 6.4 M. These solutions were heated at 40° C. for 0, 10, 30, 60, 120, 165, 280 and 320 minutes. After heating, 18 μL each of the solution was extracted from each sample, in which 2 μL each of 10 mM of antipain (manufactured by BACHEM Co.) was added. The final concentration of antipain was 1 mM.

Added into the DaniScan was 1 μL each of the solution obtained to detect the mite allergen. The quantity of the mite extract added in one DaniScan was 0.72 μg as converted into the protein.

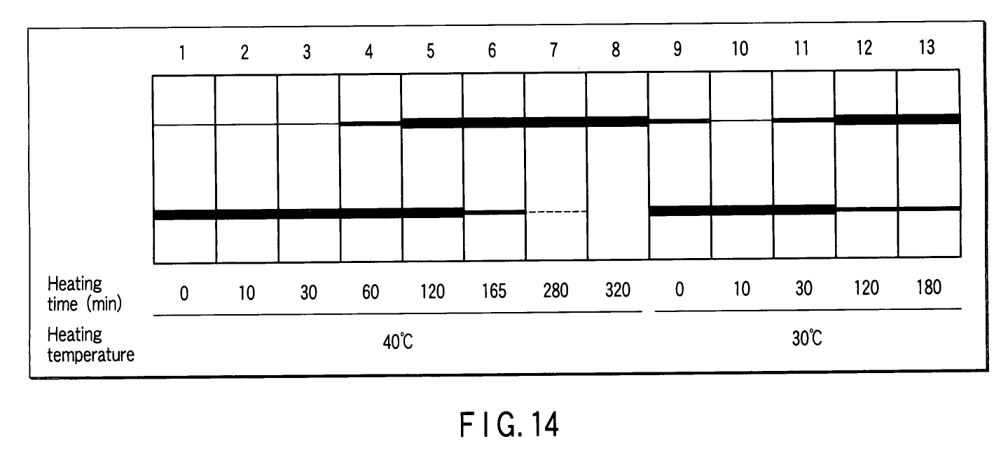

The results obtained are shown in reference numerals 1 to 8 in FIG. 14. The reference numerals 1, 2, 3, 4, 5, 6, 7, and 8 show the results of the samples heated at 40° C. for 0, 10, 30, 60, 120, 165, 280 and 320 minutes, respectively. These results show that inactivation of the allergen is possible by heating for 165 minutes under the conditions above, and allergen was almost completely inactivated by heating at 320° C.

The effect of papain by heating at 30° C. was then tested.

Added in each five micro-tubes were 20 μL of the mite preparation and 180 μL of 8 M urea-papain solution. The final concentration of urea was 7.2 M. These samples were heated at 30° C. for 0, 10, 30, 120 and 180 minutes, respectively. After heating, 18 μL each of the sample solution was extracted from each sample, and 2 μL each of 10 mM of antipain solution (manufactured by BACHEM Co.) was added in each sample. The final concentration of antipain was 1 mM.

Added in DaniScan was 1 μL each of the solutions obtained to detect the mite allergen. The quantity of the mite extract added in one DaniScan was 0.36 μg as converted into the protein.

The results obtained are shown by the reference numerals 9 to 13 in FIG. 14. The reference numerals 9, 10, 11, 12 and 13 show the results of the samples obtained by heating at 30° C. for 0, 10, 30, 120 and 180 minutes, respectively. Inactivation of the allergen was possible by heating 120 minutes under the conditions above (FIG. 14). The allergen was almost completely inactivated by heating for 320 minutes, although the result is not shown in FIG. 14.

It is evident from the examples above that the present invention provide commonly available solving methods in which the allergen comprising proteins as epitopes is inactivated. The invention is applicable to all the allergens having proteins as the epitopes.

Examples of the filter and air conditioner according to the invention will be then described with reference to the drawings.

### Example 9

Example 9 is described with reference to FIG. 1. The reference numeral 4 in the drawing show a nonwoven fabric filter for trapping the allergen. A flat heating element 5 made of stainless steel fibers and having a mesh smaller than the diameter of pollen particles (20 to 30 μm) and mite (particularly excrement of mite 10 to 40 μm) is disposed on the lower surface of the nonwoven fabric filter 4. An electric heater 6 for heating the flat heating element 5 is disposed under the heating element (registered trademark Softelex, manufactured by Teijin Co.).

The allergen inactivating filter 7 according to Example 9 comprises the nonwoven fabric filter 4 for trapping the allergen, the flat heating element 5 made of stainless steel fibbers disposed on the lower face of the nonwoven fabric filter 4 and having a mesh smaller than the diameter of pollen particles and mite, and the electric heater 6 disposed under the fiber of the flat heating element 5 for heating the heating element 5.

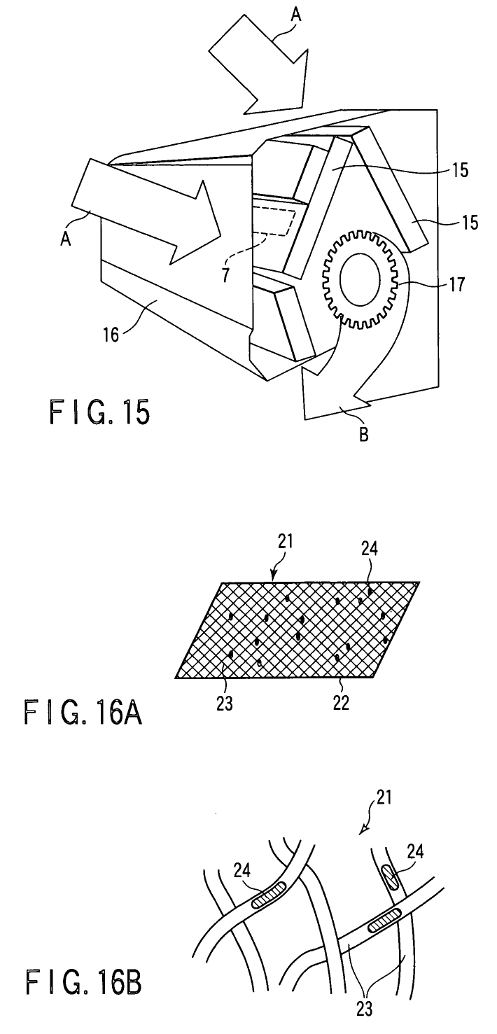

The allergen inactivating filter 7 is used at a given position of the air conditioner (see FIG. 15) to be described hereinafter with a size capable of being mounted on the air conditioner (for example, 5 cm×10 cm). The nonwoven fabric filter 4 is disposed at the inlet side of allergen-containing air (arrow A) for attaching the allergen inactivating filter 7 at the air suction port of the air conditioner. The flat heating element 5 generates heat by being heated at, for example, about 70° C. by turning the electric heater 13 of the filter 70N when the air conditioner is OFF.

The pollen particles and mites are trapped with the flat heating element 5 heated at a high temperature while allergen-containing air passes through the nonwoven fabric filter 4. Proteins of the allergen are denatured on the heating element to extinguish the activity as the allergen, and allergen-free air (arrow B) flows through the stainless steel flat heating element 5.

According to the allergen inactivating filter 7 in Example 1, adverse effect of chemicals on human bodies can be avoided since no harmful insect exterminating chemicals are not used. Instead, the quantity of the allergen may be automatically reduced only by heating the heating element at a given temperature.

### Example 10

With reference to FIG. 2, a strongly acidic cation exchange resin (not shown) is retained on the nonwoven fabric filter 9 of the allergen inactivating filter 8 according to Example 10. The strongly acidic cation exchange resin is represented by R—SO₃.H (R represents a polymer frame), and is regenerative with an acid. The strongly acidic cation exchange resin is activated before use.

According to Example 10, allergen proteins are denatured, thus the allergen is inactivated, by the effect of pH of the strongly acidic cation exchange resin retained on the nonwoven fabric filter 9 by only allowing allergen-containing air A to flow through the nonwoven fabric filter 9. Consequently, allergen-free air B is exhausted from the nonwoven fabric filter 9. Therefore, the adverse effect of chemicals on the human body may be avoided since no harmful insect exterminating chemicals are used in the allergen inactivating filter 8 in Example 10. The quantity of the allergen may be automatically reduced only by permitting allergen-containing air A to flow through the nonwoven fabric filter 22 retaining the strongly acidic cation exchange resin.

While the use of the strongly acidic cation exchange resin was described in Example 10, the resin is not restricted to the strongly acidic cation exchange resin, and the strongly basic anion exchange resin represented by the compound 1 may be also used. Preferably, the strongly basic anion exchange resin is also activated before use.

### Example 11

Example 11 is described with reference to FIG. 3. The reference numeral 10 in the drawing shows a first resin supporting member with a mesh (>50 μm) that is a size not permitting the water absorbing polymer 11 to move and not allowing the pollen particles and mites to pass through. A second resin supporting member 12, also having a mesh (>50 μm) not permitting the water absorbing polymer 13 to move and not allowing the pollen particles and mites to pass through, is disposed under the first resin supporting member 10. A water absorbing polymer layer 13 comprising the water absorbing polymer 11 is disposed by being sandwiched between the first supporting member 10 and the second supporting member 12. The water absorbing polymer 11 contains, for example, the protease with an activity of 500,000 unit/filter. The water absorbing polymer layer 13 can be formed by immersing a polymer in an enzyme solution containing the protease.

The allergen inactivating filter 14 according to Example 11 has a stricture in which the water absorbing polymer layer 13 is sandwiched between the first supporting member 10 and the second supporting member 12. Consequently, the allergen is decomposed with a protein decomposing enzyme only by allowing allergen-containing air to flow through the water absorbing polymer layer 13. Accordingly, the allergen inactivating filter 14 according to Example 11 is able to avoid adverse effect on the human body by the chemicals since no harmful insect exterminating chemicals are used. Moreover, the quantity of the allergen may be automatically reduced only by permitting allergen-containing air A to flow through the water absorbing polymer layer 13.

### Example 12

FIG. 15 shows an example in which the allergen inactivating filter according to Examples 9 to 11 is attached to the air suction port of the air conditioner. In FIG. 15, the reference numeral 15 denotes an air conditioner cooling unit, and the reference numeral 16 denotes a casing. A fun 17 and the like are disposed within the casing 16. The allergen inactivating filter 7 is disposed so that the nonwoven fabric filter as a main component of the filter 7 is positioned at the inlet of the allergen-containing air. In FIG. 15, the mark A denotes moist air, while the mark B denotes clean air.

Such an air conditioner according to Example 12 comprises the allergen inactivating filter 7 disposed at the air suction port of the air conditioner. Accordingly, the pollen particles and mites are trapped on the flat heating element 5 kept at a high temperature by heating the flat heating element 5 by turning the electric heater 6 of the filter 70N when the air conditioner is OFF, and the activity of the allergen is extinguished by denaturation of the allergen proteins. Allergen-reduced air flows through the stainless steel flat heating element 5. The power source of the air conditioner is also used for the electric heater 6.

Housing environments are remarkably improved by adding the allergen decomposing function to the air conditioner such as the air conditioner and an air cleaner.

### Example 13

FIG. 16A shows the overall view of the allergen inactivating filter, while FIG. 16B shows a partial enlarged view of FIG. 16A.

The reference numeral 21 in the drawing denotes the allergen inactivating filter for inactivating the allergen. The inactivating filter 21 comprises a filter main body 22 made of a nonwoven fabric, and an enzyme 24 directly immobilized on the fibers 23 constituting the filter main body 22. Examples of the fiber 23 include glass, rayon, cellulose, polypropylene, polyethylene terephthalate, polyacrylic acid and polyacrylamide fibers.

Physical methods as well as chemical methods may be employed for immobilizing the enzyme 24 on the fiber 23. For example, carboxylic groups of the base material are converted into azide groups, and the enzyme is immobilized on the base material by allowing the base material to chemically bonded to the enzyme with amide bonds. Functional groups are not restricted to the carboxylic group, and hydroxyl groups and amino groups may be utilized for the chemical bond. Such chemical immobilization methods have been known in the art [see Shin Jikken Kagaku Koza, Seibutu Kagaku (I), p.363-409, Maruzen Co., 1978].

According to the flat allergen inactivating filter in Example 13, the quantity of the allergen may be reduced since the filter comprises the enzyme 24 having an allergen inactivating function immobilized on the filter main body 22.

### Example 14

Referring to FIG. 17, FIG. 17 shows the main part of the flat allergen inactivating filter according to Example 14 of the invention. As shown in FIG. 17, the enzyme 24 is immobilized on a water and/or moisture absorbing carrier 25 in Example 14, and the carrier 25 is fixed on the fiber 26 using a binder (not shown). Examples of the material of the carrier 25 include synthetic materials such as polyacrylic acid, polyacrylamide and polyvinyl alcohol; natural materials such as cotton, wool, sodium alginate, mannan and agar; and regenerated materials such as rayon. Examples of the material of the fiber 6 include polyethylene (PE), polypropylene (PP), polyethylene terephthalate (PET) and polyamide (PA).

Since the flat allergen inactivating filter according to Example 14 comprises the enzyme 24 immobilized on the water and/or moisture absorbing carrier 25, which is fixed on the fiber 26 using the binder, the same effect as in Example 1 may be exhibited.

### Example 15

Referring to FIG. 18A, FIG. 18A shows the flat allergen inactivating filter according to Example 15. The inactivating filter 21 comprises a carrier 25 immobilizing a plurality of enzymes 24, and base materials 27 and 28 sandwiching the carrier 25 from above and below. Examples of the carrier 25 include polyacrylic acid, polyacrylamide and polyvinyl alcohol resins, and cotton, wool, rayon, sodium alginate, mannan and agar. The base materials 27 and 28 comprise a nonwoven fabric made of the fiber. The base material 28 under the carrier 25 is preferably made of a nonwoven fabric having a smaller mesh than the diameter of pollen particles (particle diameter: 20 to 30 μm) and mites (particularly the diameter of excrement: 10 to 40 μm) for retaining the carrier 25.

The flat allergen inactivating filter according to Example 15 has the same effect as in Example 13, since the carrier 25 immobilizing the enzyme 24 is sandwiched between the upper and lower base materials 27 and 28.

The open sandwich type flat allergen inactivating filter as shown in FIG. 18B also exhibits the same effect.

The flat allergen inactivating filter 21 shown in Examples 13 to 15 are used by being disposed in an air flow passageway of an air conditioner or the like after being housed in a casing 29 as shown in FIG. 19.

### Example 16

Referring to FIG. 20A, FIG. 20A shows a pleated allergen inactivating filter according to Example 16 of the invention. The inactivating filter 21 comprises a filter main body 22 made of fibers directly immobilizing the enzyme, and the filter main body 22 is pleated.

Since the flat inactivating filter 21 according to Example 16 comprises a filter main body 22 made of fibers directly immobilizing the enzyme, and the filter main body 22 is pleated, the filter has a lower pressure loss than that in Example 1, while the trapping ratio is enhanced by increasing contact chance with the allergen and evaporation of moisture is suppressed.

### Example 17

Referring to FIG. 20B, FIG. 20B shows a pleated allergen inactivating filter according to Example 17 of the invention. The inactivating filter 21 comprises a rod-like member 31 having a circular cross section of the bundle of fibers immobilizing the enzyme 24, and the rod-like member 31 is connected to supporting members 32 and 33 at both ends of the rod.

Since the rod-like allergen inactivating filter according to Example 17 comprises a rod-like member 31 having a circular cross section of the bundle of fibers immobilizing the enzyme 24, and the rod-like member 31 is connected to supporting members 32 and 33 at both ends of the rod, the filter has a lower pressure loss than that of the filter in Example 1, while the inactivating ability is large due to an increased amount of the immobilized enzyme and the service life of the enzyme is prolonged.

While the cross section of the rod-like member was circular in Example 17, the shape is not restricted thereto, and a triangular, rectangular or ellipsoidal shapes as well as a hollow shape are available. The direction of the rod-like member is not particularly restricted, and the rods may be oriented in a vertical or horizontal direction, or may be aligned aslant or intersected. The filter according to Example 17 may be mounted on the air conditioner by attaching at the blow-out port, or at both the suction and blow-out port, or at the position where air flows rapidly.

### Example 18

Referring to FIG. 20C, FIG. 20C shows a sponge-shaped allergen inactivating filter according to Example 18 of the invention. The inactivating filter 21 comprises the enzyme 24 immobilized on the surface of a porous material 34 such as polyurethane.

The filter according to Example 18 can exhibit the same effect as in Example 13.

### Example 19

Referring to FIGS. 21A and 21B, FIG. 21A is provided for illustrating the water feed method to the allergen inactivating filter according to Example 19 of the invention, and FIG. 21B shows an enlarged cross section taken along the line 21B-21B in FIG. 21A. The heater, insulation material and water absorbing polymer are not shown in FIG. 21A for the convenience of descriptions.

A capillary type allergen inactivating filter 41 is used in the apparatus in Example 19. The filter 41 comprises a plurality of rod-like members 42 elongating in a horizontal direction, and the rods are supported with supporting members 43 and 44 as terminals at both ends. The water absorbing polymer 47 is provided with interposition of an insulation material 46 at the outer circumference of a heater (iron core) 45. The heater 45 of the rod-like member 42 is electrically connected to the supporting members 43 and 44 at both ends. The lower end of the filter 41 is immersed in water in a water tank 48 having a slit (not shown) along the longitudinal direction (right and left directions in the drawing) at the upper part, and water in the water tank 48 is soaked up into the filter 41 from the slit by a capillary action. A heater power source 49 is connected to the supporting members 43 and 44. The reference numeral 41a in the drawing shows a gap between the rod-like members 42.

Since the lower end of the allergen inactivating filter 41 is immersed in water in the water tank 48 according to Example 19, water in the water tank 48 is soaked up into the filter 41 from the slit to enable moisture required for exhibiting the effect of the enzyme on the filter 41 to be always replenished from the water tank 48.

### Example 20

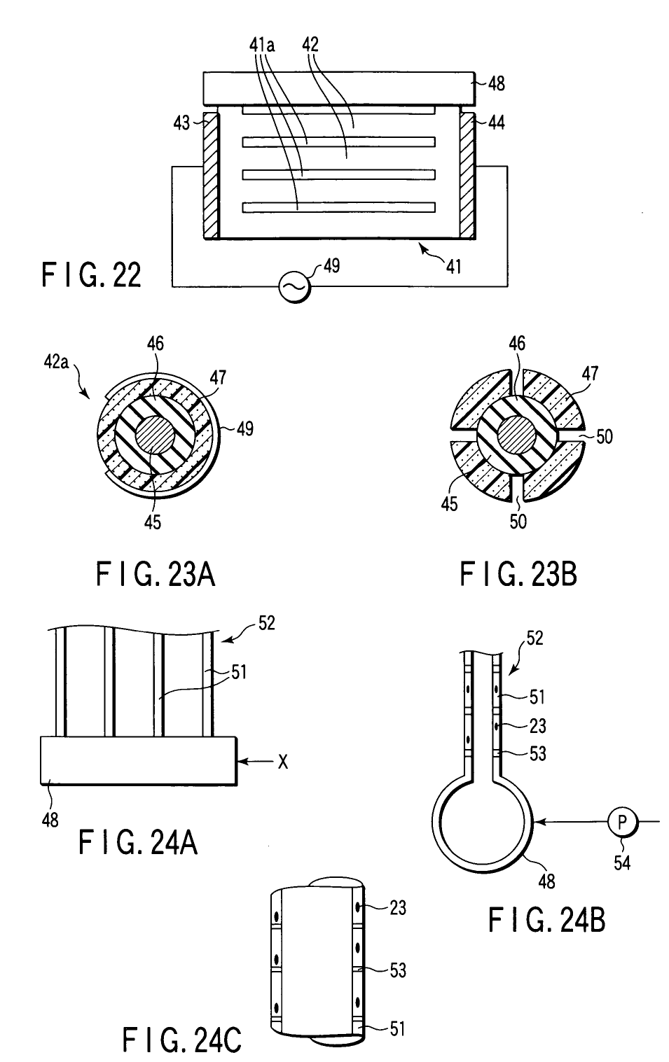

FIG. 22 shows a method for feeding water to the allergen inactivating filter according to Example 20 of the invention. The same members as in FIG. 21 are given the same reference numerals as in FIG. 21, and descriptions thereof are omitted.

The method in FIG. 22 in Example 20 has a similar structure as that in Example 19, except that the water tank 48 is disposed at the upper part of the filter 41.

Since the water tank 48 is disposed at the upper part of the filter 41 in Example 20, water in the water tank 48 is readily soaked down into the filter 41 by the water's own weight as compared with Example 19 to enable water to enable replenishment of water into the filter 41 to be more reliably effected.

While the water absorbing polymer was provided by aligning the rod-like members constituting the filter around the heater with interposition of an insulation material in Examples 19 and 20, the arrangement is not restricted thereto. For example, the rod-like member 42a may have a cover 49 having an opening at the air passage side at the outer circumference of the water absorbing polymer 27 as shown in FIG. 23A, or the rod-like member 42b may comprise many holes 50 in the water absorbing polymer 47 as shown in FIG. 23B. The cover 49 is provided for preventing excess moisture from being evaporated from the filter 41 in order to maintain the water retaining property for a long period of time. Otherwise, a hydrophilic polymer may be disposed around the water absorbing polymer in another rod-like member, and a cover having an opening at the air passage side at the outer circumference of the rod-like member, although this arrangement is not shown in the drawing.

While the water absorbing material is usually a polymer, it is needless to say that any water absorbing materials may be used except the polymers.

### Example 21

Referring to FIGS. 24A to 24C, FIG. 24A shows a method for feeding water to the allergen inactivating filter according to Example 21 of the invention, FIG. 24B is a perspective view taken along the line X in FIG. 24A, and FIG. 24C is a partially enlarged view of FIG. 24B. The same reference numerals are given to the same members as those in FIGS. 16 and 21, and descriptions thereof are omitted.

The reference numeral 51 in the drawing shows a moisture permeating tube reaching from an opening (not shown) of the water tank 48 filled with water to the water portion. The allergen inactivating polymer filter 52 comprises a plurality of moisture permeating tube 51 with an appropriate distance apart with each other. The material of the moisture permeating tube 51 is a hollow fiber made of cellulose acetate or regenerated cellulose. Polytetrafluoroethylene (registered trademark Goatex, manufactured by Goa and Associates Co.) is also available. The enzyme 23 is immobilized on the moisture permeating tube 51 as shown in FIG. 24B, while micron order many holes 53 are provided for permitting water in the water tank 48 to seep. A pump 54 as pressurizing means for pressurizing the space within the water tank 48 is connected to the water tank 48.

Since the pump 54 is connected to the water tank 48 according to Example 21, the amount of water sent to the moisture permeating tube 51 can be controlled by adjusting the pressure in the water tank 48.

While the pump is used as the pressurizing means in Example 21, the pressurizing means is not restricted to the pump. For example, a heater for heating water in the water tank may be provided around the water tank, and the water content in the moisture permeating tube may be controlled by evaporating water through the holes on the moisture permeating tube by heating water.

### Example 22

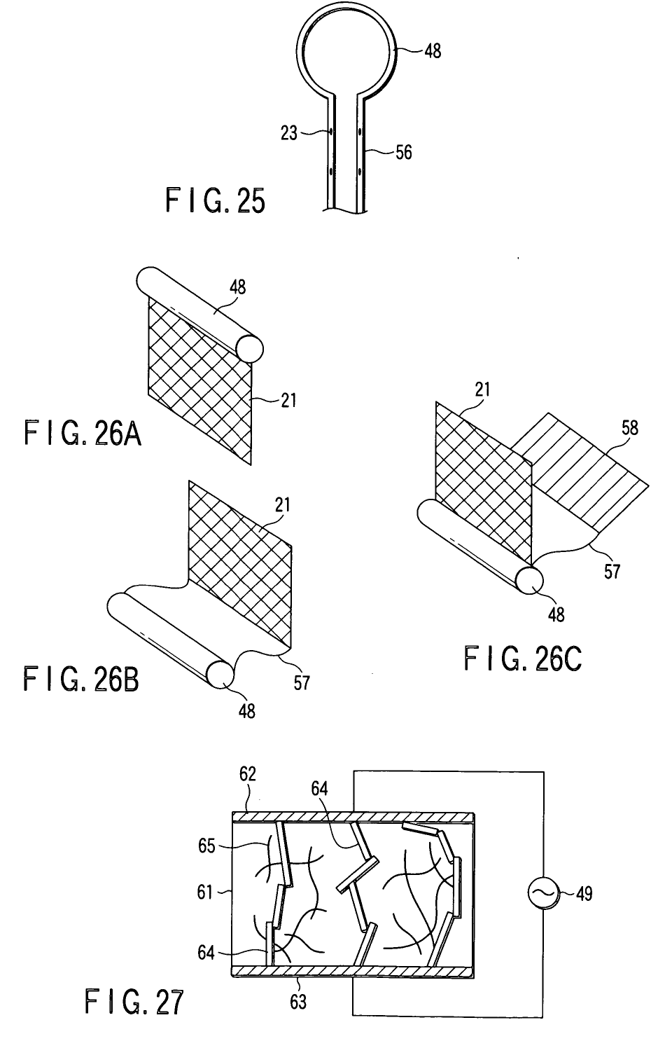

Referring to FIG. 25, FIG. 25 schematically shows a method for feeding water to the allergen inactivating filter according to Example 22 of the invention. The same embers as those in FIGS. 16 and 21 are given the same reference numerals, and descriptions thereof are omitted.

The water tank 48 is disposed above the allergen inactivating filter 55 in Example 22. The filter 55 comprises a plurality of hollow fibers 56 immobilizing the enzyme 23, and the hollow fiber is connected to the water tank 48.

According to Example 22, water in the water tank 48 is transferred on the surface of the fiber by water's own weight from the water tank 48 through the hollow fiber 56 to activate the enzyme. The service life of the filter 55 is prolonged by suppressing the amount of evaporation of water to its minimum.

### Example 23

FIGS. 26A to 26C are schematic views for describing a method of feeding water to the flat allergen inactivating filter according to Example 23 of the invention. The same members as those in FIGS. 16 and 21 are given the same reference numerals, and descriptions thereof are omitted.

While the methods for feeding water to the filter comprising the rod-like or hollow members were described in Examples 18 to 23, the method for feeding water to the flat filter is shown in Example 23. Practically, the methods are as shown in (1) to (3) below.

(1) As shown in FIG. 26A, a water tank 48 having a slit (not shown) in the longitudinal direction is used, the filter 21 is disposed so that the upper end thereof is inserted into the slit of the water tank 48, and water is fed from the water tank 48 to the filter 21 by the water's own weight.

(2) As shown in FIG. 26B, the slit of the water tank 48 is connected to the filter 21 via a nonwoven fabric 57, and water in the water tank 48 is fed to the filter 21 through the nonwoven fabric 57 by a capillary action.

(3) As shown in FIG. 26C, the water tank 48 is disposed under the filter 21, and an evaporator (or a drain pot) 38 is connected to water in the water tank 48 via the nonwoven fabric 57. Water from the evaporator 58 is fed to the filter 21 through the nonwoven fabric 57 and water tank 48.

While the nonwoven fabric 57 was used in FIGS. 26A and 26B, the method is not restricted thereto, and any materials may be used so long as they have the capillary action.

In the method described in (1) above, water in the water tank 48 is transferred from the water tank 48 to the filter 21 by the water's own weight, and activates the enzyme immobilized on the filter 21. In the method (2), water in the water tank 48 is transferred to the filter 21 from the water tank 48 through the nonwoven fabric 57, and activates the enzyme immobilized on the filter 21. In the method (3), water accumulated in the evaporator or drain pot is effectively utilized for activating the enzyme immobilized on the filter 21.

### Example 24

Example 24 describes a method for heating the allergen activating filter according to the invention. FIG. 27 shows the heating method for a mixed woven allergen inactivating filter 61, and supporting members 62 and 63 that serve as electrodes are disposed at the upper and lower parts of the filter 61, respectively. The filter 61 comprises a conductive filter 64, a non-conductive polymer 65, and the enzyme (not shown). Conductive particles may be used in place of the conductive fiber 64.

A voltage is applied to the conductive fiber 64 between the supporting members 62 and 63 for heating the filter 61 using an alternating current source 49 arranged as described above. The mixed woven allergen inactivating filter 61 is heated by the method as described above. The filter is heated because, since particles of the mites and allergens one trapped on the filter tend to be scattered into the air by passing through the filter with the lapse of time, the allergen is required to be inactivated by heating the filter.

### Example 25

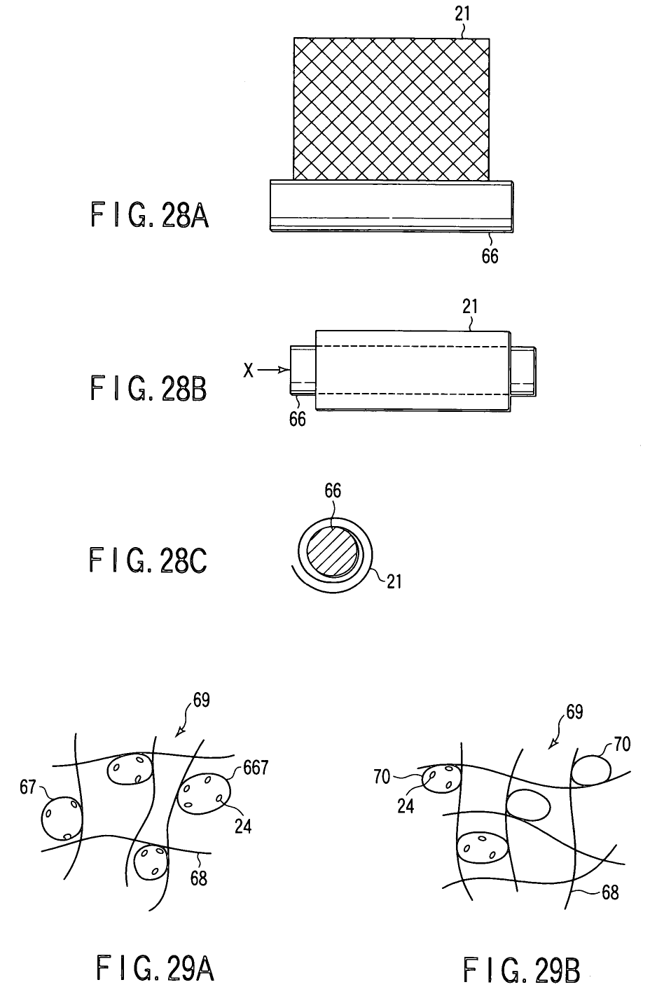

Referring to FIGS. 28A to 28C, FIG. 28A illustrates the filter in use, FIG. 28B illustrates the filter in waiting, and FIG. 28C shows a perspective view taken along the arrow X in FIG. 28B. FIGS. 28A, 2B and 28C show a method of heating a roll cake type (having a swirled cross section) allergen inactivating filter.

As shown in FIG. 28A, only the allergen is trapped on the filter 21 when the filter 21 is in use in, for example, an air conditioner. On the other hand, the filter 21 is rolled on the heater 66 when the operation of the air conditioner is stopped as shown in FIGS. 28B and 28C, and the filter is heated by turning the heater ON. Therefore, it is possible to effectively inactivate the allergen trapped on the filter 21. Since the filter 66 is wound around the heater in the roll cake type filter, the filter 21 can be effectively heated.

The heating method for the filter is not restricted to the methods described in FIGS. 27 and 28. For example, the heater may be disposed at the center of the rod-like water absorbing polymer constituting the filter as shown in FIG. 28A, and the filter is heated with the heater while a cover having a partial opening is attached at the outside of the water absorbing material (core type heating). Otherwise, a hydrophilic material is disposed around the rod-like water absorbing material, and the filter is heated while the heater is disposed at a part around the hydrophilic material (ecternal heating type, not shown). An infrared light or a microwave may be used for heating.

### Example 26

FIGS. 29A and 29B illustrates an allergen inactivating filter having added functions. FIG. 29A shows a filter integrated with an adsorbent, while FIG. 29B shows a filter mixed with the adsorbent. The same members as those in FIG. 16 are given the same reference numerals, and descriptions thereof are omitted.

In FIG. 29A, the reference numeral 67 denotes adsorbent particles immobilizing the enzyme 24. The materials of the adsorbent 67 are not particularly restricted, and examples thereof include inorganic porous materials such as activated charcoal, zeolite, sepiolite, kaolinite and montmorillonite. The adsorbent particles 67 are retained on the nonwoven fabric 68 as a filter main body to constitute the allergen inactivating filter 69.

In FIG. 29B, the allergen inactivating filter 69 is constructed by fixing a carrier 70 immobilizing the enzyme 24 and adsorbent particles 67 on the nonwoven fabric 68.

Since the adsorbent particles 67 is immobilized in addition to the allergen inactivating enzyme 24 according to Example 26, a deodorizing effect as well as the allergen inactivating effect may be obtained.

While the adsorbent particles are used in addition to the allergen inactivating enzyme in Example 26, the additive is not restricted thereto. Additives such as antibiotic and antifungal photocatalyst particles (such as TiO₂, ZnO and CdS), aroma particles and negative ion generating particles may be also used.

### Example 27

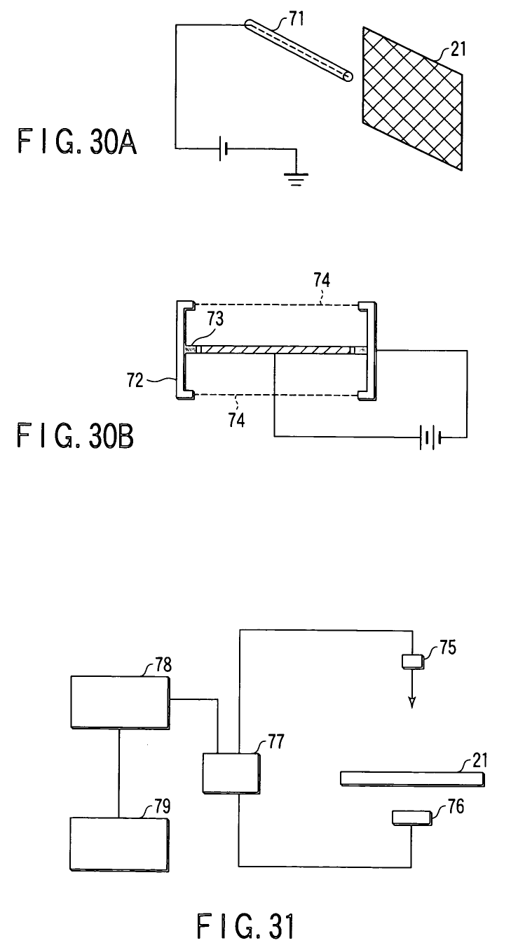

FIGS. 30A and 30B shows addition of a dust-preventive function to the allergen inactivating filter by applying a voltage.

In FIG. 30A, a corona discharge is generated using a wire electrode 71 on the filter 21 immobilizing the enzyme in order to electrify the particles in the vicinity of the wire electrode 71.

The filter 21 is supported with a supporting member 72 with interposition of an insulation material 73 in FIG. 30B, and metal meshes 74 are disposed above and below the filter 21 with a distance apart from the filter 21 to electrify the particles passing through the metal mesh 74.

According to Example 27, the particles can be trapped on the inversely electrified filter having an inverse electric potential.

### Example 28

FIG. 31 is a schematic diagram illustrating a device for monitoring the service life of the allergen inactivating filter.

A light emitting body 75 is disposed just above the filter 21. A light receiving body 76 is disposed just under the filter 21. The light emitting body 75 and light receiving body 76 are electrically connected to a controller 77. Output means 78 for exporting a filter exchange timing and display means 79 such as a lamp are put into sequential electric connected to the controller 77. The controller 77 is provided for retention, measurement, comparison, recognition and prescription as described below. For example, the input to the light receiving body 76 is measured while the output of the light emitting body 75 is maintained at a specified value. The controller recognizes that the fiber diameter is reduced when the input level is increased above a given level by comparing the output level with the input level, and instruct the exchange timing on display means 79.

The actual width of the fibers 23 constituting the filter 21 is detected in the apparatus in FIG. 31, and the measured width is compared with the width as a standard of exchange. The controller 77 informs of exchange of the filter 21 by sending a signal to the display means 79 from the output means 78 based on the result of measurements.

The service life of the filter 21 can be reliably recognized by the apparatus in Example 28 by providing the display means 79 for displaying the exchange timing by a comparison of the actual width of the fiber with the fiber as a measure of the exchange timing, even when the allergen inactivating filter 21 to an air conditioner.

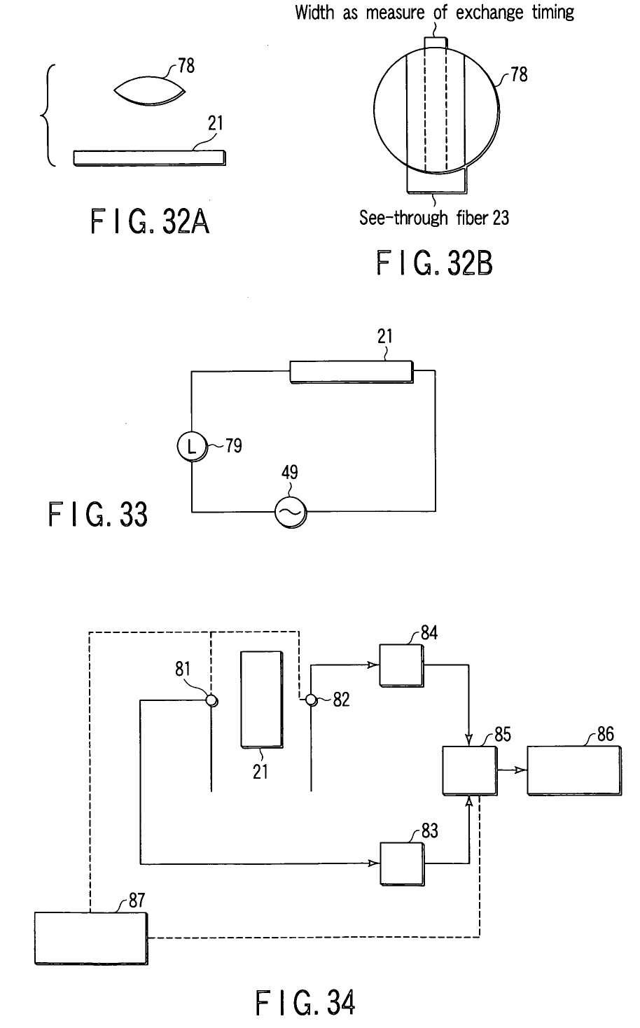

The methods shown in FIGS. 32A and 32B may be used in place of the method shown in FIG. 31. FIG. 32A shows the arrangement of a lens 78 relative to the filter 21, and FIG. 32B shows an example of the timing for replacing the filter 21. The width of the fiber may be readily recognized by the naked eye only by providing the lens 78. The fiber 23 is seen to be wide as shown in FIG. 32B when water is fed to the filter 21. However, since the width of the fiber 23 becomes small when the volume water replenished in the water tank is reduced, the filter 21 is exchanged when, for example, the fiber looks like the broken line in FIG. 32B.

While the service life of the filter was confirmed by monitoring the with of the fiber in Example 28, the method is not restricted thereto. For example, the service life of the filter may be confirmed by bonding a member that changes its color by wetting, by providing a thermometer, or by using an antibody kit.

For example, a closed circuit comprising a lamp 79 and an alternating current source 49 are provided on the filter 21 as shown in FIG. 33 so that the lamp 79 lights depending on the changes of the water content in the filter 21. A scale that displays the magnitude of an electric current may be provided in place of the lamp. Since conductivity of the filter changes depending on the water content.

Alternatively, the service life of the filter may be confirmed by a device shown in FIG. 34. The reference numerals 81 and 82 in FIG. 34 denote an inlet side sensor and outlet side sensor provided at the inlet and outlet sides, respectively, of the filter 21. Conversion means 83 for converting the output of the sensor 81 into the concentration (efficacy) of the allergen is connected to the inlet side sensor 81, and conversion means 84 for converting the output of the sensor 82 into the concentration (efficacy) of the allergen is connected to the outlet side sensor 82.

Determination means 85 for determining the allergen removing efficiency and filter service life at that time by comparing the inlet side sensor output with the outlet side sensor outlet is connected to the conversion means 83 and 84. The determination means 85 is connected to a display circuit 86 for displaying with optical means such as a lamp and liquid crystal panel. It is not needed to say that acoustic means such as a buzzer and speaker may be used in place of the optical means. The inlet side sensor 81 and outlet side sensor 82 are connected to operation mode control means 87, which is connected to the determination means 85.

Since the inlet side and outlet side sensors 81 and 82 are connected to the inlet side and outlet side of the filter 21 in the apparatus according to FIG. 34, respectively, the allergen removing efficiency and filter service life at the time are determined by comparing the outputs of these sensors to enable the service life of the filter 21 to be more reliably confirmed.

### Example 32

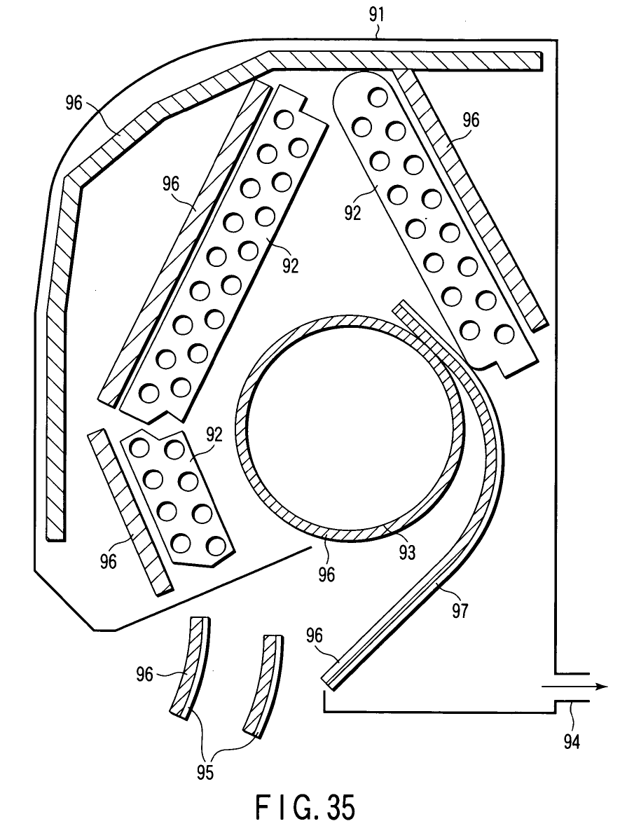

With reference to FIG. 35, Example 32 shows an allergen inactivating filter integrated into the air conditioner according to Example 13.

The reference numeral 91 denotes a casing in FIG. 25. A plurality of components such as a heat exchanger 92 and a fan 93 of the air conditioner cooling unit are disposed in the casing 91. Components such as a drain 94 are disposed from the inside to the outside of the casing 91, and a louver 95 is disposed at the air exhaust port. The allergen inactivating filter is disposed at the air flow passageway 96 of the air conditioner in Example 32. The air flow passageway 96 includes the front of the heat exchanger, inner wall of the casing, fan blade and louver. The reference numeral 97 in FIG. 35 denotes the casing.

Since the allergen inactivating filter is disposed in the air flow passageway 96 of the air conditioner in Example 32, pollen particles and mites can be reliably trapped to enable the amount of the allergen to be more reduced as compared with usual air conditioners.

### Example 33

A filter having no heating mechanism with a heater was described in Example 32. An allergen inactivating filter having a heating mechanism such as a heater as shown in FIG. 21 is applied to the air conditioner in Example 33. The heating mechanism involves a function of an allergen removing operation mode, which is actually comprises a heater, a supporting member as an electrode for supporting the heater, and an electric power source.

For effecting the allergen removing operation mode, the filter is constructed to be able to be wound. The contact area of the filter with air is usually increased by extending the filter to aggressively trap the allergen. When a considerable amount of the allergen has been trapped, on the other hand, the filter is wound up to reduce the volume in order to accelerate the allergen inactivating reaction by permitting heat to be efficiently transferred. The filter is extended again thereafter to set a cycle of allergen trapping cycle. However, the filter may remain extended when the heater is a radiation type instead of a conduction type.

In the case of Example 33, the filter is heated at, for example, about 70° C. by turning the heater ON when the air conditioner is OFF. Consequently, the allergen derived from pollen particles and mites are trapped with the filter while allergen-containing air flows through the filter, and the activity of the allergen is extinguished by effectively denaturing the allergen proteins by an enzyme having an enhanced activity by being heated with the heater. Accordingly, air containing less allergens flows through the filter. In other words, the allergen derived particles discharged from the filter again, if any, are harmless since the activity as the allergen has been extinguished.

Since the amount of generation of the allergen changes depending on seasons, the allergen removing operation mode may be appropriately selected. It is needless to say that the air conditioner is used in a usual operation mode in the seasons when the allergen is hardly generated.

While the allergen inactivating filter has been applied to the air conditioner in Examples 32 and 33, the application is not restricted thereto, and the same effect may be obtained by applying to the air cleaner, dehumidifier, dryer, humidifier and ventilator.

Detection of the allergen, allergen and inactivating method will be described hereinafter.

[Method and Material]

(1) Detection of Allergen

The inventors employed the measuring method using the antibody as described above.

(2) Allergen

The mite extract-Df was used in this test as in the foregoing example. Dissolved in 2.5 mL of PBS was 10 mg of the mite extract to obtain a solution with a concentration of 4 mg/mL. The solution obtained is called as a mite preparation, and was used by diluting to an appropriate concentration with the PBS solution.

[Inactivating Method]

### Example 34

### Measurement of Allergen

The relative amount of the mite allergen in the sample was measured using DaniScan. A calibration curve of the mite allergen in the mite extract was prepared at first for confirming availability of mite scanning for quantitative analysis.

The allergen in the solution containing the mite preparation diluted with the PBS solution was detected using DaniScan (manufactured by Asahi Food and Health Care Co.).

DaniScan is a kit that is able to simply detect the mite allergen by taking advantage of an antigen-antibody reaction. The test sample was added to a sampling part 2 of a sampler 1 provided in DaniScan shown in FIG. 6. A band 3 reflecting the amount of the mite allergen in the test sample is obtained by adding a developer in the sample. The bands of DaniScan are shown by “bold solid line”, “intermediately bold solid line”, “fine solid line”, “broken line” and “no indication”, and the luminous intensity was schematically confirmed in four grades in the order of “bold solid line”>“intermediately bold solid line”>“fine solid line”>“broken line”>“no indication”, but not shown in the figure.

The amount of the mite allergen contained in the sample was confirmed by calculating peak ratios between the luminous intensity peak value (T) ascribed to the mite antibody bonded to the mite antigen and the luminous intensity peak value (C) ascribed to the mite antibody not bonded to the mite antigen in the mite extract according to the following equation. FIG. 7 is a graph showing the position on DaniScan and luminous intensity. The peak value (T) and peal value (C) in the equation correspond to the peaks denoted by [C] and [T], respectively, in FIG. 7.

The equation for calculating the peak ratio from the luminous intensities of the peak C and peak T is:
Peak ratio (%)=[T/(T+C)]×100
The amount of the mite allergen is small when C>T, while the amount of the mite allergen is large in the sample when T>C.

FIG. 6 is a schematic illustration of DaniScan showing the pattern in which a mite allergen positive band 's shown (when the amount of the allergen is large). The position C shows a band derived from the mite antigen not bonded to the mite antigen, while the position T shows a band derived from the mite antigen bonded to the mite antigen. FIG. 8 shows the calibration curve of the mite allergen contained in the mite extract.

### Example 35

### Inactivation with Protease and Denaturing Agent 1

The following experiment was carried out for proving the mite allergen inactivating action when the protease and denaturing agent were used together.

Added in 18.0 μL of the mite preparation was 22.0 μL of sterilized water. This solution was used as a control.

### Example 36

### Inactivation with Protease and Denaturing Agent 2

Added in disposable tubes with a volume of 1.5 mL was 18.0 μL of the mite preparation, and an SDS solution was added in the tubes so that the final concentrations in the tubes were 0.05, 0.1, 0.3. 1.0 and 2.0% to prepare reaction solutions, respectively. Added to the solutions was 2 μL each of a pfu protease S solution to prepare reaction solutions with a total volume of 40 μL.

The reaction solutions prepared as described above were heat-treated on a block heater at 40° C. The tubes were transferred on ice at the timing 10, 30, 60, 120 and 240 minutes after the start of the heat treatment, and 2 μL each of phenylmehtylsulfonyl fluoride (PMSF) as an inhibitor of the protease was added to each reaction solution to stop the reaction.

A solution containing 18.0 μL of the mite preparation and 22.0 μL of sterilized water was used as a control in the experiment.

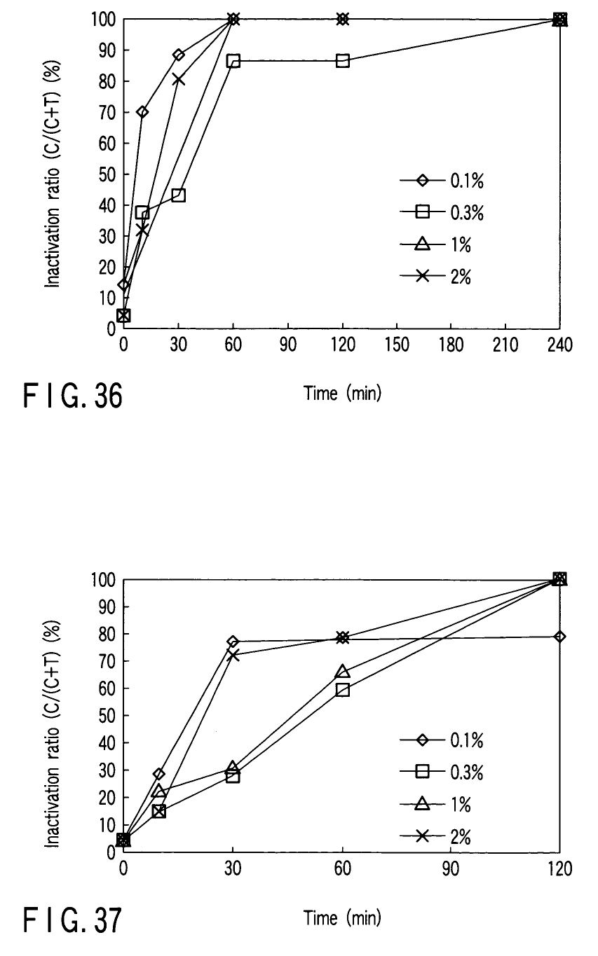

Spotted on DaniScan was 1 μL each of the sample solution after the reaction to detect the mite allergen. The bands appeared on DaniScan were investigated, and DaniScan was subjected to an image processing, and each peak was analyzed. The results are shown in FIG. 36.

The results showed that the mite allergen was inactivated by treating with SDS with a concentration of 0.1% or more. The reaction was the fastest when the SDS concentration is 0.1%, and about 90% of the allergen was inactivated by a heat treatment at 40° C. for 30 minutes. The mite allergen could be also completely inactivated by a prolonged heat treatment at 40° C. when the concentration of SDS is higher.

### Example 37

### Inactivation with Protease and Denaturing Agent 3

Added in disposable tubes with a volume of 1.5 mL was 18.0 μL of the mite preparation, and an SDS solution was added in the tubes so that the final concentrations in the tubes are 0.1, 0.3. 1.0 and 2.0%, respectively. Added to the solutions was 2 μL each of a pfu protease S solution to prepare reaction solutions with a total volume of 40 μL.

The reaction solutions prepared as described above were heat-treated on a block heater at 30° C. The tubes were transferred on ice at the timing 10, 30, 60, 120 and 240 minutes after the start of the heat treatment, and 2 μL each of phenylmehtylsulfonyl fluoride (PMSF) as an inhibitor of the protease was added to each reaction solution to stop the reaction.

A solution containing 18.0 μL of the mite preparation and 22.0 μL of sterilized water was used as a control in the experiment.

Spotted on DaniScan was 1 μL each of the sample solution after the reaction to detect the mite allergen. The band appeared on DaniScan was investigated. DaniScan was subjected to an image processing, and each peak was analyzed. The results are shown in FIG. 37.

While a little longer time was necessary before inactivation as compared with the results obtained by heating at 40° C., the mite allergen was completely inactivated by treating with SDS with a concentration of 0.3% or more at 30° C. for 120 minutes. Although progress of decomposition of the mite allergen was observed at an SDS concentration of 0.1%, the allergen was not completely inactivated by heating at 30° C. for 120 minutes.

### Example 38

### Inactivation with Protease and Denaturing Agent 4

Added in disposable tubes with a volume of 1.5 mL was 18.0 μL of the mite preparation, and an SDS solution was added in the tubes so that the final concentrations in the tubes are 0.1, 0.3. 1.0 and 2.0%, respectively. Added in the solution was 2 μL of a papain PBS solution prepared in a concentration of 0.2 g/mL to prepare reaction solutions with a total volume of 40 μL.

The reaction solutions prepared as described above were heat-treated on a block heater at 40° C. The tubes were transferred on ice at the timing 10, 30, 60, 120 and 240 minutes after the start of the heat treatment, and 2 μL each of antipapain (manufactured by Roche Co., 1 tablet/2 mL) as an inhibitor of the protease was added to each reaction solution to stop the reaction.

A solution containing 18.0 μL of the mite preparation and 22.0 μL of sterilized water was used as a control in the experiment.

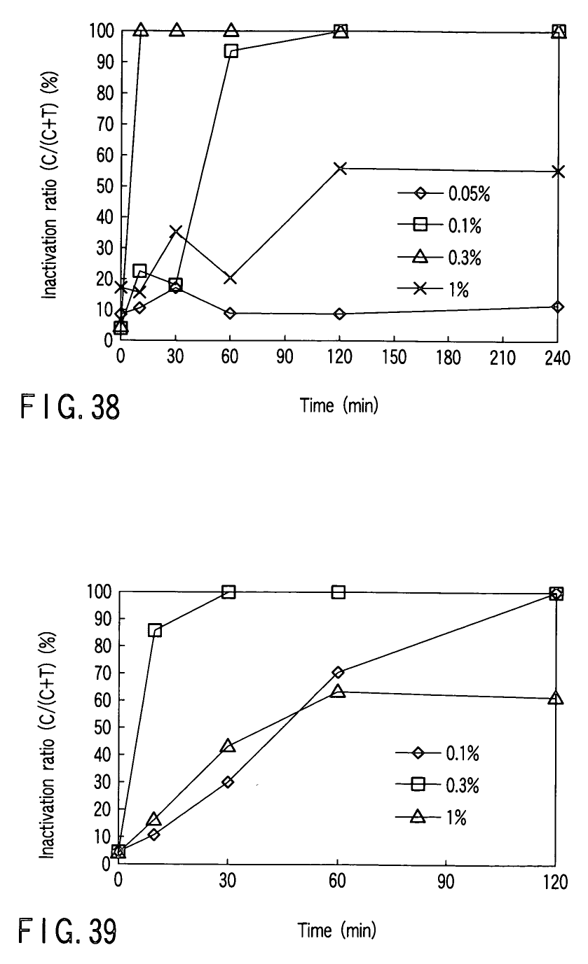

Spotted on DaniScan was 1 μL each of the sample solution after the reaction to detect the mite allergen. The band appeared on DaniScan was investigated. DaniScan was subjected to an image processing, and each peak was analyzed. The results are shown in FIG. 38.

The reaction was not advanced at an SDS concentration of 0.05% as in the example using pfu protease S, the mite allergen was inactivated at a concentration of 0.1% or more. The reaction was the fastest at an SDS concentration of 0.3%. The mite allergen was completely inactivated by heat treating at 40° C. for 10 minutes at an SDS concentration of 0.3%. However, the mite allergen was not decomposed at an SDS concentration of 1.0% or more. This is considered to be ascribed to denaturation of the enzyme (papain) itself.

### Example 39

### Inactivation with Protease and Denaturing Agent 5

Added in disposable tubes with a volume of 1.5 mL was 18.0 μL of the mite preparation, and an SDS solution was added in the tubes so that the final concentrations in the tubes are 0.1, 0.3 and 1.0%, respectively. Added in the solution was 2 μL of a papain PBS solution prepared in a concentration of 0.2 g/mL to prepare reaction solutions with a total volume of 40 μL.

The reaction solutions prepared as described above were heat-treated on a block heater at 30° C. The tubes were transferred on ice at the timing 10, 30, 60, 120 and 240 minutes after the start of the heat treatment, and 2 μL each of antipapain (manufactured by Roche Co., 1 tablet/2 mL) as an inhibitor of the protease was added to each reaction solution to stop the reaction.

A solution containing 18.0 μL of the mite preparation and 22.0 μL of sterilized water was used as a control in the experiment.

Spotted on DaniScan was 1 μL each of the sample solution after the reaction to detect the mite allergen. The band appeared on DaniScan was investigated. DaniScan was subjected to an image processing, and each peak was analyzed. The results are shown in FIG. 39.

No inactivation of the mite allergen was observed at an SDS concentration of 1.0% as in the results shown in Example 38. This is considered to be ascribed to denaturation of the enzyme itself. It took a little longer time before inactivation in this example as compared with the results heat treated at 40° C., and complete inactivation was confirmed by heat treating at 30° C. at an SDS concentration of 0.3%.

The results above showed that inactivation of the allergen is possible within a short period of time at a temperature close to the room temperature by using the method according to the invention. While mite was used as the allergen in the examples above, the antigen is not restricted to the mite allergen, and any allergens comprising proteins as epitopes may be inactivated at a temperature close to the room temperature according to the method of the invention.

[Examples of Filter and Air Conditioning]

Examples of the filter and air conditioner according to the invention will be described with reference to the drawings.

### Example 40

Example 40 is described with reference to FIG. 3. However, the inactivating filter in this example is a little different from the filter in Example 11, it will be described again. In Example 40, the first supporting member 10 has a mesh (>50 μm) with a size not allowing the water absorbing polymer 11 to move and not permitting pollen particles and mites to permeate. The second supporting member 12 also has a mesh (>50 μm) with a size not allowing the water absorbing polymer 11 to move and not permitting pollen particles and mites to permeate. The water absorbing polymer layer 13 comprises the water absorbing polymer 11. The water absorbing polymer 11 contains the protease with a 500,000 unit/filter and the denaturing agent. The water absorbing polymer layer 13 is formed, for example, by impregnating the polymer with a solution containing the protease and denaturing agent.

The allergen inactivating filter 14 according to Example 40 has a structure in which the water absorbing polymer layer 13 is sandwiched between the first supporting ember 10 made of resin and the second supporting member 12 each made of resin. The allergen is decomposed with a protein decomposing enzyme merely by allowing allergen-containing air A to flow through the water absorbing polymer layer 13. Accordingly, since no harmful insect exterminating chemicals are used in the allergen inactivating filter 14 in Example 40, adverse effects on human bodies may be avoided while the amount of the allergen is automatically reduced by merely allowing allergen-containing air A to flow through the water absorbing polymer layer 13.

### Example 41

The allergen inactivating filter 14 according to Example 41 is attached to the air suction port of the air conditioner as described in Example 12. Members such as the air conditioner cooling unit 15, casing 16 and fan 17 are as described in Example 12.

The allergen inactivating filter 14 is disposed at the air suction port of the air conditioner according to Example 41. Accordingly, the pollen particles and mites are trapped on a flat heating body maintained at a high temperature by heating the flat heating body by turning an electric heater of the filter ON when the air conditioner is OFF. The allergen protein is denatured there to extinguish the activity as the allergen. Allergen-reduced air flows through the stainless flat heating body. The electric heater uses the electric power source of the air conditioner.

Living environments may be remarkably improved by adding the allergen decomposing function of the invention to the air conditioning apparatus such as the air conditioner and air cleaner.

The invention provides a method for specifically excluding the allergen at a temperature near the room temperature within a shorter period of time.

[Method for Inactivating Allergen as Pollen Disease Causing Substance]

The invention will be described in more detail with reference to examples.

(Material)

The experiment was carried out in the examples of the invention using the cedar pollen as the allergen that is considered to be most prevailing causes of the pollen disease patients for describing the effect of the invention. Specifically, the cedar extract and cedar antigen were used as the allergens of cedar.

(Cedar Extract)

A commercially available cedar extract was used as the cedar pollen extract, which was cedar extract-Cj manufactured by Cosmo Bio Co. This cedar extract was homogenized by stirring the cedar pollen with sodium hydrogen carbonate (pH 8) followed by dialyzing against a borate buffer solution. The cedar extract was used as a cedar preparation by suspending in purified water at a concentration of 1 mg/mL.

(Cedar Antigen)

Cedar antigens Cryj-1 (abbreviated as Cj-1 hereinafter) and Cryj-2 (abbreviated as Cj-2 hereinafter) were manufactured by Hayashibara Biochemical laboratories, Inc., and was used in a concentration of 100 μg/mL.

Analysis by electrophoresis is shown in Examples 42 to 45 below.

### Example 42

### Inactivation of Cedar Extract by Heat

(1) Sample 1 (Control)

Added in a 1.5 mL volume disposable tube were 22.5 μL of the cedar preparation and 2 μL of water, followed by adding 0.5 μL of a phenylmethylsulfonyl fluoride solution (abbreviated as PMSF solution hereinafter as a protease inhibitor; final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 1.

(2) Sample 2 (Heat Treatment of Cedar Extract 1)

Added in a 1.5 mL volume disposable tube were 22.5 mL of the cedar preparation and 2 μL of water, followed by heating the solution at 80° C. for 20 minutes. A PMSF solution (0.5 μL) was added therein (a final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 2.

(3) Sample 3 (Heat Treatment of Cedar Extract 2)

Added in a 1.5 mL volume disposable tube were 22.5 μL of the cedar preparation and 2 μL of water, followed by heating the solution at 65° C. for 120 minutes. A PMSF solution (0.5 μL) was added therein (a final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 3.

(4) Electrophoresis

The electrophoresis samples 1 to 3 prepared by the methods in (1) to (3), respectively, were analyzed by electrophoresis. NuPage™ MOPS buffer kit for Bis-tris gel was used as an electrophoresis buffer solution, and 15 mL each of samples 1 to 3 prepared above were added in each lane. The protein was stained after electrophoresis using simply Blue™ SafeStain manufactured by Invitrogen Co. The gel was decolorized according to the prescription of the manufacturer.

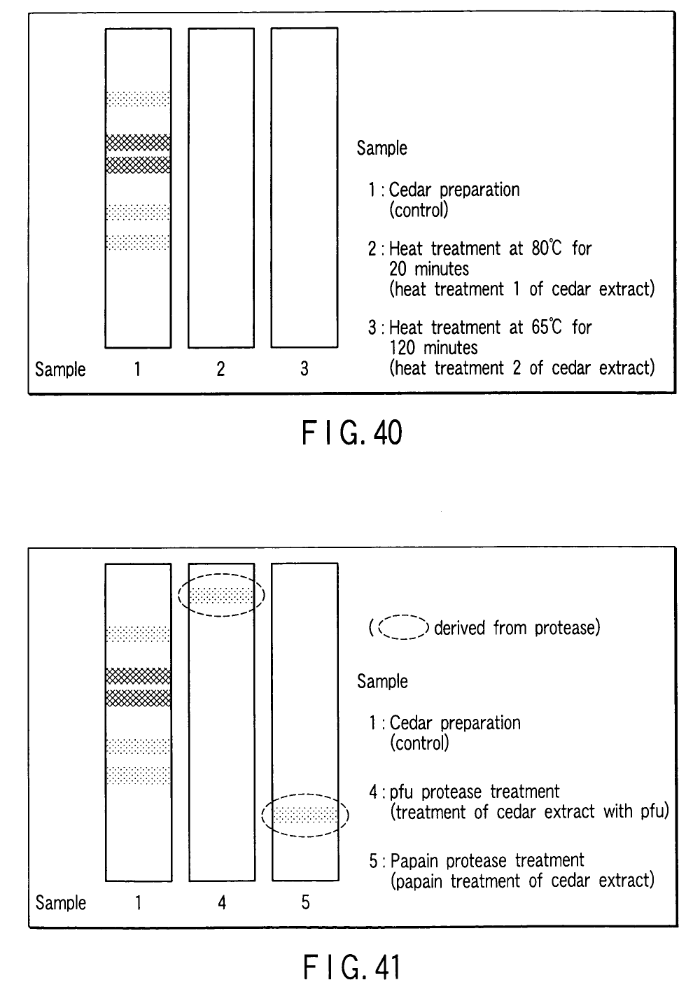

The results are shown in FIG. 40. FIG. 40 shows that a plurality of bands observed in sample 1 were not observes in samples 2 and 3 after heating. This means that most of the proteins derived from the cedar were denatured by heating at 80° C. for 20 minutes and at 65° C. for 120 minutes (FIG. 40).

### Example 43

### Inactivation of Cedar Extract by Protease

(1) Sample 1 (Control)

Added in a 1.5 mL volume disposable tube were 22.5 μL of the cedar preparation and 2 μL of water, followed by adding 0.5 μL of a PMSF solution (a final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 1.

(2) Sample 4 (Treatment of Cedar Extract with pfu)

Added in a 1.5 mL volume disposable tube were 22.5 μL of the cedar preparation and 2 μL of a pfu protease S solution (manufactured by Takara Bio Co.), followed by heating the solution at 80° C. for 20 minutes. A PMSF solution (0.5 μL) was added therein (a final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 4.

(3) Sample 5 (Papain Treatment of Cedar Extract)

Added in a 1.5 mL volume disposable tube were 27.0 μL of the cedar preparation and 3 μL (0.2 g/mL) of an edible papain solution (manufactured by Nagase Chemtex Co.; abbreviated as papain hereinafter), followed by heating the solution at 40° C. for 15 minutes. 3 μL of an inhibitor cocktail solution (a protease inhibitor, manufactured by Rocje Co.; 1 tablet/ml) was added therein (a final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 5.

(4) Electrophoresis

The electrophoresis samples 1, 4 and 5 prepared by the methods in (1) to (3), respectively, were analyzed by electrophoresis. NuPage™ MOPS buffer kit for Bis-tris gel was used as an electrophoresis buffer solution, and 15 μL each of samples 1, 4 and 5 prepared above were added in each lane. The protein was stained after electrophoresis using simply Blue™ SafeStain manufactured by Invitrogen Co. The gel was decolorized according to the prescription of the manufacturer.

The results are shown in FIG. 41. FIG. 41 shows that a plurality of bands observed in sample 1 by the two kinds of the proteases were not observed in samples 4 and 5. Only the band originating from the protease (the band surrounded by a intermediately bold broken line in FIG. 41) was observed in samples 4 and 5 at the position where no band had been observed in sample 1. Accordingly, it was shown that most of the cedar proteins had been decomposed by the treatment using the two kinds of the proteases (FIG. 41).

### Example 44

### Inactivation of Cedar Antigen Cryj-1 with Protease

(1) Sample 6 (Control)

Added in a 1.5 mL volume disposable tube were 22.5 μL of the Cj-1 solution and 2 μL of water, followed by adding 0.5 μL of a PMSF solution (a final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 6.

(2) Sample 7 (Treatment of Cedar Antigen with pfu)

Added in a 1.5 mL volume disposable tube were 22.5 μL of the Cj-1 solution and 2 μL of water, and the solution was heated at 80° C. for 20 minutes, followed by adding 0.5 μL of the PMSF solution (final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 7.

(3) Electrophoresis

The electrophoresis samples 6 and 7 prepared by the methods in (1) and (2), respectively, were analyzed by electrophoresis. NuPage™ MOPS buffer kit for Bis-tris gel was used as an electrophoresis buffer solution, and 15 μL each of samples 6 and 7 prepared above were added in each lane. The protein was stained after electrophoresis using simply Blue™ SafeStain manufactured by Invitrogen Co. The gel was decolorized according to the prescription of the manufacturer.

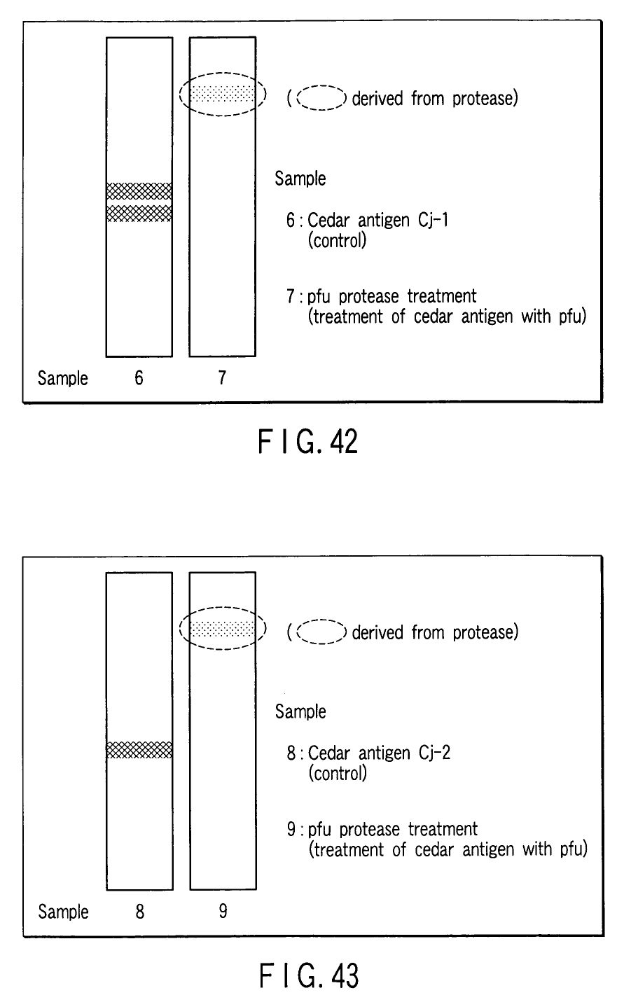

The results are shown in FIG. 42. FIG. 42 shows that two bands observed in sample 6 by the proteases treatment were not observed in sample 7. Only the band originating from the protease (the band surrounded by a intermediately bold broken line in FIG. 42) was observed. Accordingly, it was shown that most of the cedar antigen Cj-1 protein had been decomposed by the protease treatment (FIG. 42).

### Example 45

### Inactivation of Cedar Antigen Cryj-2 with Protease

(1) Sample 8 (Control)

Added in a 1.5 mL volume disposable tube were 22.5 μL of the Cj-2 solution and 2 μL of water, followed by adding 0.5 μL of a PMSF solution (a final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 8.

(2) Sample 9 (Treatment of Cedar Antigen with pfu)

Added in a 1.5 mL volume disposable tube were 22.5 μL of the Cj-2 solution and 2 μL of the pfu splution, and the solution was heated at 80° C. for 20 minutes, followed by adding 0.5 μL of the PMSF solution (final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 9.

(3) Electrophoresis

The electrophoresis samples 8 and 9 prepared by the methods in (1) and (2), respectively, were analyzed by electrophoresis. NuPage™ MOPS buffer kit for Bis-tris gel was used as an electrophoresis buffer solution, and 15 μL each of samples 8 and 9 prepared above were added in each lane. The protein was stained after electrophoresis using simply Blue™ SafeStain manufactured by Invitrogen Co. The gel was decolorized according to the prescription of the manufacturer.

The results are shown in FIG. 43. FIG. 43 shows that the band observed in sample 8 was disappeared by the proteases treatment, and only the band originating from the protease (the band surrounded by a intermediately bold broken line in FIG. 43) was observed in sample 9 (FIG. 43). Accordingly, it was shown that most of the cedar antigen Cj-2 protein had been decomposed by the protease treatment (FIG. 43).

Examples 46 to 51 below show analysis by electrophoresis and Western blotting.

Inactivation of the allergen by a combination of electrolysis and Western blotting after treating the allergen by the same methods as in Examples 42 to 45 were measured, and the results are shown below.

The allergen is a protein as described above. Accordingly, an ability as an antigen may be extinguished by decomposition and/or denaturation of the protein. The effective inactivating method of the allergen being able to understand by biochemical knowledge is as described above.

Simplified measurement methods (methods by taking advantage of dot blotting and DaniScan™) had been used in these methods. Accordingly, details of inactivation of the allergen were not clear. Allergens contained in one causing substance, for example, the allergens contained in mites or pollens comprises allergens classified into a number of classes. Accordingly, it is crucial to confirm the possibility of inactivation with respect to individual allergens.

The inventors have disclosed a method for measuring the activities of individual allergens in Japanese Patent Application No. 2002-106834. Electrophoresis of the protein and Western blotting are combined in this method, by which allergens that serve as epitopes are separated by electrophoresis of the protein. Decomposition and/or denaturation of the protein, if any, can be determined using the concentration of the band for detecting the presence of the protein as indices. For more detailed analysis, the sample is subjected to Western blotting by taking advantage of an antigen-antibody reaction to determine whether or not the existing substance has allergenic property. Elucidation of inactivation of the allergen treated by the method according to the invention by the method as described above will be described below.

### Example 46

### Inactivation of Cedar Extract by Heat

(1) Sample 1 (Control)

Added in a 1.5 mL volume disposable tube were 22.5 μL of the cedar preparation and 2 μL of water, followed by adding 0.5 μL of a PMSF solution (a final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 1.

(2) Sample 2 (Heat Treatment of Cedar Extract 1)

Added in a 1.5 mL volume disposable tube were 22.5 μL of the cedar preparation and 2 μL of water, and the solution was heated at 80° C. for 20 minutes, followed by adding 0.5 μL of the PMSF solution (final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 2.

(3) Sample 3 (Heat Treatment of Cedar Extract 2)

Added in a 1.5 mL volume disposable tube were 22.5 μL of the cedar preparation and 2 μL of water, and the solution was heated at 65° C. for 120 minutes, followed by adding 0.5 μL of the PMSF solution (final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 3.

(4) Electrophoresis

The electrophoresis samples 1 to 3 prepared by the methods in (1) to (3), respectively, were analyzed by electrophoresis. NuPage™ MOPS buffer kit for Bis-tris gel was used as an electrophoresis buffer solution, and 15 μL each of samples 1 to 3 prepared above were added in each lane for electrophoresis.

(5) Western Blotting

After the electrophoresis in (4) above, the proteins were electrically transferred on a pre-treated PVDF membrane. XcellII™ Blot Module manufactured by Invitrogen Co. was used for pre-treatment and transfer of the PVDF membrane according to the prescription of the manufacturer.

Cj-1 antibody (manufactured by Cosmo Bio Co.; rabbit whole serum) as a primary antibody was used by diluting 5,000 times for detecting the allergen by Western blotting in this example. WesternBreeze Chemiluminescent Detection System, Anti Rabbit (registered trademark, manufactured by Invitrogen Co.) was used as a secondary antibody, and blocking and detection reagent according to the prescription of the manufacturer. A luminescent reagent was added to the PVDF membrane after treating as described above, and the membrane was photographed on a Polaroid film using an instant ECL mini-camera (registered trademark, manufactured by Amersham Pharmacia) to detect the protein.

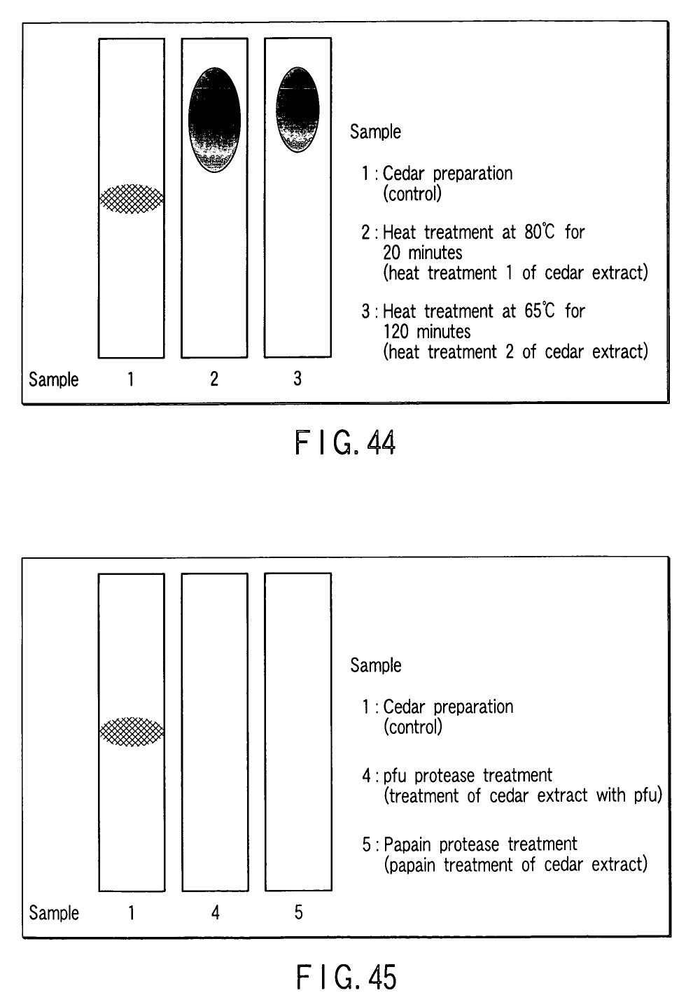

The results are shown in FIG. 44. FIG. 44 shows that the band observed in sample 1 was disappeared in samples 2 and 3, instead a broad band spreading in a large area above the band described above was observed. This band may be ascribed to denaturation of the cedar protein. Accordingly, it was suggested from the result that Cj-1 allergen was inactivated by heating (FIG. 44).

### Example 47

### Inactivation of Cedar Extract with Protease

(1) Sample 1 (Control)

Added in a 1.5 mL volume disposable tube were 22.5 μL of the cedar preparation and 2 μL of water, followed by adding 0.5 μL of a PMSF solution (a final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 1.

(2) Sample 4 (Treatment of Cedar Extract with pfu)

Added in a 1.5 mL volume disposable tube were 22.5 μL of the cedar preparation and 2 μL of the pfu solution, and the solution was heated at 80° C. for 20 minutes, followed by adding 0.5 μL of the PMSF solution (final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 4.

(3) Sample 5 (Treatment of Cedar Extract with Papain)

Added in a 1.5 mL volume disposable tube were 27.0 μL of the cedar preparation and 3 μL (0.2 g/mL) of edible purified papain (manufactured by Nagase Chemtex Co.; abbreviated as papain hereinafter), and the solution was heated at 40° C. for 15 minutes, followed by adding 3 μL of an inhibitor cocktail solution. NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 5.

(4) Electrophoresis

The electrophoresis samples 1, 4 and 5 prepared by the methods in (1) to (3), respectively, were analyzed by electrophoresis. NuPage™ MOPS buffer kit for Bis-tris gel was used as an electrophoresis buffer solution, and 15 μL each of samples 1, 4 and 5 prepared above were added in each lane for electrophoresis.

(5) Western Blotting

After the electrophoresis in (4) above, the proteins were electrically transferred on a pre-treated PVDF membrane. XcellII™ Blot Module manufactured by Invitrogen Co. was used for pre-treatment and transfer of the PVDF membrane according to the prescription of the manufacturer.

Cj-1 antibody as a primary antibody was used by diluting 5,000 times for detecting the allergen by Western blotting in this example. WesternBreeze Chemiluminescent Detection System, Anti Rabbit (registered trademark, manufactured by Invitrogen Co.) was used as a secondary antibody, and blocking and detection reagent according to the prescription of the manufacturer. A luminescent reagent was added to the PVDF membrane after treating as described above, and the membrane was photographed on a Polaroid film using an instant ECL mini-camera (registered trademark) to detect the protein.

The results are shown in FIG. 45. FIG. 45 shows that the band observed in sample 1 was disappeared in samples 4 and 5. Accordingly, it was suggested from the result that Cj-1 allergen was completely inactivated by the protease (FIG. 45).

### Example 48

### Inactivation of Cedar Extract by Heat

(1) Sample 1 (Control)

Added in a 1.5 mL volume disposable tube were 22.5 μL of the cedar preparation and 2 μL of water, followed by adding 0.5 μL of a PMSF solution (a final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 1.

(2) Sample 2 (Treatment of Cedar Extract by Heat)

Added in a 1.5 mL volume disposable tube were 22.5 μL of the cedar preparation and 2 μL of water, and the solution was heated at 80° C. for 20 minutes, followed by adding 0.5 μL of the PMSF solution (final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 2.

(3) Electrophoresis

The electrophoresis samples 1 and 2 prepared by the methods in (1) and (2), respectively, were analyzed by electrophoresis. NuPage™ MOPS buffer kit for Bis-tris gel was used as an electrophoresis buffer solution, and 15 μL each of samples 1 and 2 prepared above were added in each lane. The protein was stained after electrophoresis using simply Blue™ SafeStain manufactured by Invitrogen Co. The gel was decolorized according to the prescription of the manufacturer.

(4) Western Blotting

After the electrophoresis in (3) above, the proteins were electrically transferred on a pre-treated PVDF membrane. XcellII™ Blot Module manufactured by Invitrogen Co. was used for pre-treatment and transfer of the PVDF membrane according to the prescription of the manufacturer.

Cj-2 antibody as a primary antibody was used by diluting 5,000 times for detecting the allergen by Western blotting in this example. WesternBreeze Chemiluminescent Detection System, Anti Rabbit (registered trademark, manufactured by Invitrogen Co.) was used as a secondary antibody, and blocking and detection reagent according to the prescription of the manufacturer. A luminescent reagent was added to the PVDF membrane after treating as described above, and the membrane was photographed on a Polaroid film using an instant ECL mini-camera (registered trademark) to detect the protein.

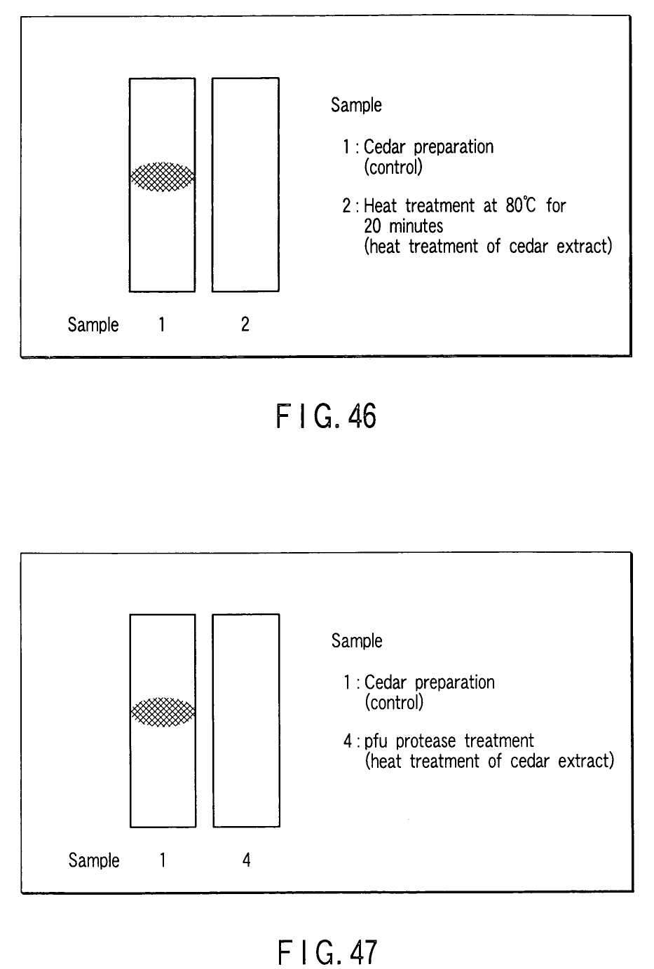

The results are shown in FIG. 46. FIG. 46 shows that the band observed in sample 1 was disappeared in sample 2. Accordingly, it was suggested from the result that Cj-2 allergen was completely inactivated by the protease (FIG. 46).

### Example 49

### Inactivation of Cedar Extract with Protease

(1) Sample 1 (Control)

Added in a 1.5 mL volume disposable tube were 22.5 μL of the cedar preparation and 2 μL of water, followed by adding 0.5 μL of a PMSF solution (a final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 1.

(2) Sample 4 (Treatment of Cedar Extract with pfu)

Added in a 1.5 mL volume disposable tube were 22.5 μL of the cedar preparation and 2 μL (0.2 unit) of the pfu solution, and the solution was heated at 80° C. for 20 minutes, followed by adding 0.5 μL of the PMSF solution (final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 4.

(3) Electrophoresis

The electrophoresis samples 1 and 4 prepared by the methods in (1) and (2), respectively, were analyzed by electrophoresis. NuPage™ MOPS buffer kit for Bis-tris gel was used as an electrophoresis buffer solution, and 15 μL each of samples 1 and 4 prepared above were added in each lane.

(4) Western Blotting

After the electrophoresis in (3) above, the proteins were electrically transferred on a pre-treated PVDF membrane. XcellII™ Blot Module manufactured by Invitrogen Co. was used for pre-treatment and transfer of the PVDF membrane according to the prescription of the manufacturer.

Cj-2 antibody as a primary antibody was used by diluting 5,000 times for detecting the allergen by Western blotting in this example. WesternBreeze Chemiluminescent Detection System, Anti Rabbit (registered trademark, manufactured by Invitrogen Co.) was used as a secondary antibody, and blocking and detection reagent according to the prescription of the manufacturer. A luminescent reagent was added to the PVDF membrane after treating as described above, and the membrane was photographed on a Polaroid film using an instant ECL mini-camera (registered trademark) to detect the protein.

The results are shown in FIG. 47. FIG. 47 shows that the band observed in sample 1 was disappeared in sample 4. Accordingly, it was suggested from the result that Cj-2 allergen was completely inactivated by the protease treatment (FIG. 47).

### Example 50

### Inactivation of Cedar Antigen Cryj-1 with Protease

(1) Sample 6 (Control)

Added in a 1.5 mL volume disposable tube were 22.5 μL of the Cj-1 solution and 2 μL of water, followed by adding 0.5 μL of a PMSF solution (a final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 6.

(2) Sample 7 (Treatment of Cedar Antigen with pfu)

Added in a 1.5 mL volume disposable tube were 22.5 μL of the Cj-1 solution and 2 μL (0.2 unit) of the pfu solution, and the solution was heated at 80° C. for 20 minutes, followed by adding 0.5 μL of the PMSF solution (final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 7.

(3) Electrophoresis

The electrophoresis samples 6 and 7 prepared by the methods in (1) and (2), respectively, were analyzed by electrophoresis. NuPage™ MOPS buffer kit for Bis-tris gel was used as an electrophoresis buffer solution, and 15 μL each of samples 6 and 7 prepared above were added in each lane.

(4) Western Blotting

After the electrophoresis in (3) above, the proteins were electrically transferred on a pre-treated PVDF membrane. XcellII™ Blot Module manufactured by Invitrogen Co. was used for pre-treatment and transfer of the PVDF membrane according to the prescription of the manufacturer.

Cj-1 antibody as a primary antibody was used by diluting 5,000 times for detecting the allergen by Western blotting in this example. WesternBreeze Chemiluminescent Detection System, Anti Rabbit (registered trademark, manufactured by Invitrogen Co.) was used as a secondary antibody, and blocking and detection reagent according to the prescription of the manufacturer. A luminescent reagent was added to the PVDF membrane after treating as described above, and the membrane was photographed on a Polaroid film using an instant ECL mini-camera (registered trademark) to detect the protein.

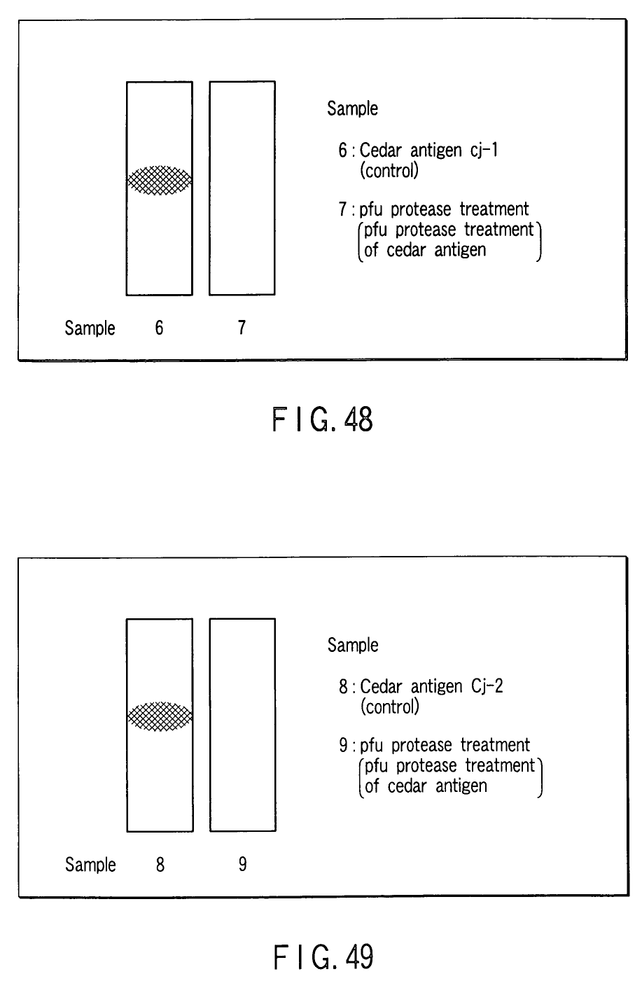

The results are shown in FIG. 48. FIG. 48 shows that the band observed in sample 6 was disappeared in sample 7. Accordingly, it was suggested from the result that Cj-1 allergen was completely inactivated by the protease treatment (FIG. 48).

### Example 51

### Inactivation of Cedar Antigen Cryj-2 with Protease

(1) Sample 8 (Control)

Added in a 1.5 mL volume disposable tube were 22.5 μL of the Cj-2 solution and 2 μL of water, followed by adding 0.5 μL of a PMSF solution (a final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 8.

(2) Sample 9 (Treatment of Cedar Antigen with pfu)

Added in a 1.5 mL volume disposable tube were 22.5 μL of the Cj-2 solution and 2 μL of the pfu solution, and the solution was heated at 80° C. for 20 minutes, followed by adding 0.5 μL of a PMSF solution (a final concentration of PMSF was 4 mM). NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 9.

(3) Electrophoresis

The electrophoresis samples 8 and 9 prepared by the methods in (1) and (2), respectively, were analyzed by electrophoresis. NuPage™ MOPS buffer kit for Bis-tris gel was used as an electrophoresis buffer solution, and 15 mL each of samples 8 and 9 prepared above were added in each lane.

(4) Western Blotting

After the electrophoresis in (3) above, the proteins were electrically transferred on a pre-treated PVDF membrane. XcellII™ Blot Module manufactured by Invitrogen Co. was used for pre-treatment and transfer of the PVDF membrane according to the prescription of the manufacturer.

Cj-2 antibody as a primary antibody was used by diluting 5,000 times for detecting the allergen by Western blotting in this example. WesternBreeze Chemiluminescent Detection System, Anti Rabbit (registered trademark, manufactured by Invitrogen Co.) was used as a secondary antibody, and blocking and detection reagent according to the prescription of the manufacturer. A luminescent reagent was added to the PVDF membrane after treating as described above, and the membrane was photographed on a Polaroid film using an instant ECL mini-camera (registered trademark) to detect the protein.

The results are shown in FIG. 49. FIG. 49 shows that the band observed in sample 8 was disappeared in sample 9. Accordingly, it was suggested from the result that Cj-2 allergen was completely inactivated by the protease treatment (FIG. 49).

Examples of the air conditioner comprising a pollen allergen inactivating part are described in Examples 52 to 55 below.

### Example 52

Example 52 is described with reference to FIG. 1. The allergen inactivating filter according to Example 52 comprises, as described in Example 9, a nonwoven fabric filter 4 for trapping the allergen, and a stainless steel flat heating element 5 disposed under the nonwoven fabric 4 and having a mesh with a size smaller than the diameter of pollen particles and mites, and an electronic heater 6 disposed under the flat heating element 5 for heating the flat heating element 5.

The allergen inactivating filter 7 is used by being attached at a given position of the air conditioner (see FIG. 15) with a seize (for example, 5 cm×10 cm) as a size capable of mounting on the sir conditioner. When the allergen inactivating filter 7 is attached on the air suction port of the air conditioner, the nonwoven fabric 4 is disposed at the inlet side of allergen-containing air (the arrow A). The flat heating element 5 is heated, for example, at about 70° C. by turning the electric heater 6 of the filter 70N when the air conditioner is OFF.

While allergen-containing air flows through the nonwoven fabric filter 4, the pollen particles and mites are trapped on the flat heating element 5 maintained at a high temperature. The allergen protein is denatured to extinguish an activity as the allergen, and allergen-free air (arrow B) flows through the stainless steel flat heating element 5.

Since no harmful insect exterminating chemicals are used according to the allergen inactivating filter 7 in Example 52, adverse effects on human bodies may be avoided while the amount of the allergen is automatically reduced by merely heating the heating element at a predetermined temperature.

### Example 53

with reference FIG. 2, the allergen inactivating filter 8 according to Example 53 comprises, as described in Example 10, a strongly acidic cation exchange resin (not shown) retained on the nonwoven fabric filter 9. The strongly acidic cation exchange resin is represented by R—SO₃.H (R represents a polymer frame), and is reproducible using an acid. The strongly acidic cation exchange resin is activated before use.

According to Example 53, the allergen protein is denatured by the effect of pH of the strongly acidic cation exchange resin retained on the nonwoven fabric filter 9 merely by allowing allergen-containing air A to flow through the nonwoven fabric filter 9. Consequently, allergen-free air B is exhausted from the nonwoven fabric filter 9. Accordingly, since no harmful insect exterminating chemicals are used according to the allergen inactivating filter 8 in Example 53, adverse effects on human bodies may be avoided while the amount of the allergen is automatically reduced by merely allowing allergen-containing air A to flow through the nonwoven fabric filter 9 retaining the strongly acidic cation exchange resin.

While use of the strongly acidic cation exchange resin was described in Example 53, a strongly basic anion exchange resin shown in the compound 1 may be used. The strongly basic anion exchange resin is also preferably activated before use.

### Example 54

Example 54 will be described with reference to FIG. 3. The reference numeral 10 in the drawing denotes a first resin supporting member having a mesh (>50 μm) that is a size not permitting the water absorbing polymer 11 to move and not allowing the pollen particles and mites to pass through. The water absorbing polymer layer 13 comprising the water absorbing polymer 11 is disposed by being sandwiched between the first supporting member 10 and the second supporting member 12. The second supporting member 12, also having a mesh (>50 μm) with a size that does not permit the water absorbing polymer 11 to move and permits the pollen particles and mites to pass through, is disposed under the first supporting member 10. The water absorbing polymer 11 contains, for example, the protease with a concentration of 500,000 units/filter. The water absorbing polymer layer 13 can be formed by impregnating the polymer with an enzyme solution containing the protease.

The allergen inactivating filter 14 according to Example 54 comprises the water absorbing polymer layer 13 sandwiched between the first supporting member 10 and the second supporting member 12 each made of a resin. Allergen is decomposed with the protein decomposing enzyme by merely allowing allergen-containing air A to flow through the water absorbing polymer layer 13. Accordingly, since no harmful insect exterminating chemicals are used according to the allergen inactivating filter 14 in Example 54, adverse effects on human bodies may be avoided while the amount of the allergen is automatically reduced by merely allowing allergen-containing air A to flow through the water absorbing polymer layer 13.

### Example 55

The allergen inactivating filter 7 according to Example 55 is attached at the air suction port of the air conditioner as described in Example 12. The members such as the air conditioner cooling unit 15, casing 16 and fan 17 are as described in Example 12.

The air conditioner according to Example 55 comprises the allergen inactivating filter 7 attached at the air suction port of the air conditioner. Accordingly, the pollen particles and mites are trapped on a flat heating element 12 maintained at a high temperature by heating the flat heating element 12 by turning an electric heater 13 of the filter 70N when the air conditioner is OFF. The allergen is denatured there to extinguish the activity as the allergen. Allergen-free air flows through the stainless flat heating element 12. The electric heater 13 uses the electric power source of the air conditioner.

Living environments may be remarkably improved by adding the allergen decomposition mechanism of the invention to the air conditioning apparatus such as the air conditioner and air cleaner. The invention provides a method for specifically excluding the allergen at a temperature near the room temperature within a shorter period of time.

Examples 56 to 63 below show the effect of an acid and alkali.

### Example 56

### Inactivation of Cedar Extract with Acid

(1) Sample 10 (Control)

Added in a 1.5 mL volume disposable tube were 10 μL of the cedar preparation and 20 μL of water. NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 10.

(2) Sample 11 (Treatment of Cedar Extract with HCl)

Added in a 1.5 mL volume disposable tube were 10 μL of the cedar preparation and 10 μL of 5 N HCL solution, followed by incubating at 60° C. for 60 minutes. Incubation was stopped by neutralizing the reaction solution by adding 10 μL of 5 N sodium hydroxide solution.

NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 11.

(3) Electrophoresis

The electrophoresis samples 10 and 11 prepared by the methods in (1) and (2), respectively, were analyzed by electrophoresis. NuPage™ MOPS buffer kit for Bis-tris gel was used as an electrophoresis buffer solution, and 15 μL each of samples 10 and 11 prepared above were added in each lane. The protein was stained after electrophoresis using simply Blue™ SafeStain manufactured by Invitrogen Co. The gel was decolorized according to the prescription of the manufacturer.

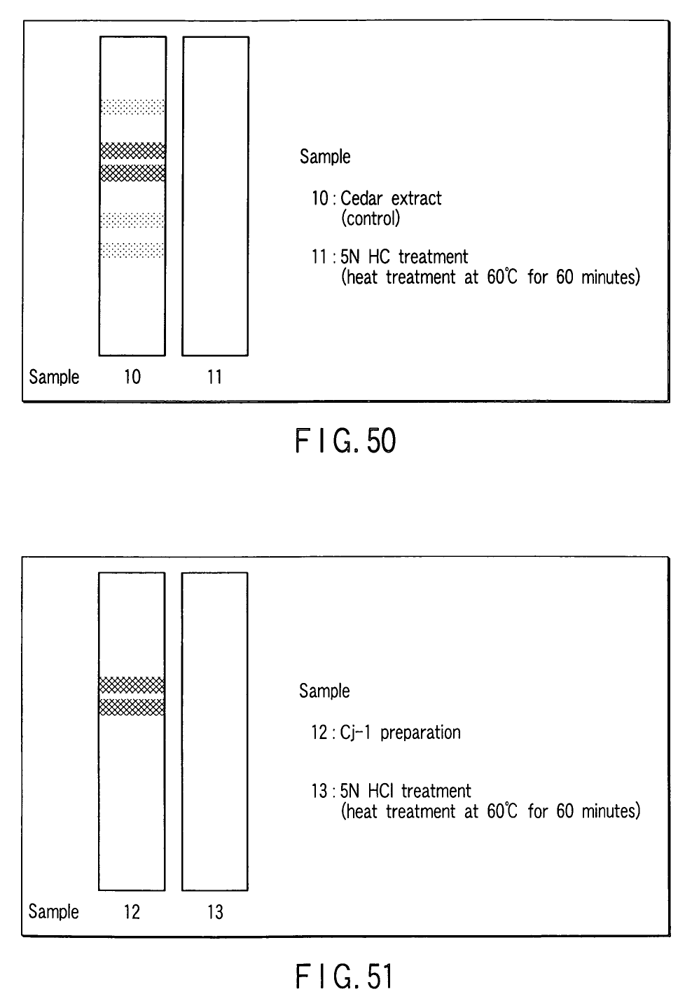

The results are shown in FIG. 50. FIG. 50 shows that the band observed in sample 10 and ascribed to the cedar pollen allergen was disappeared in Sample 11. Accordingly, It was shown that most of the proteins derived from cedar was denatured (FIG. 50).

### Example 57

### Inactivation of Cedar Antigen Crj-1 with Acid

(1) Sample 12 (Control)

Added in a 1.5 mL volume disposable tube were 10 μL of the Cj-1 solution and 20 μL of water. NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 12.

(2) Sample 13 (Treatment of Cedar Antigen with HCl)

Added in a 1.5 mL volume disposable tube were 10 μL of the Cj-1 solution and 10 μL of 5 N HCL solution, followed by incubating at 60° C. for 60 minutes. Incubation was stopped by neutralizing the reaction solution by adding 10 μL of 5 N sodium hydroxide solution. NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 13.

(3) Electrophoresis

The electrophoresis samples 12 and 13 prepared by the methods in (1) and (2), respectively, were analyzed by electrophoresis. NuPage™ MOPS buffer kit for Bis-tris gel was used as an electrophoresis buffer solution, and 15 μL each of samples 12 and 13 prepared above were added in each lane. The protein was stained after electrophoresis using simply Blue™ SafeStain manufactured by Invitrogen Co. The gel was decolorized according to the prescription of the manufacturer.

The results are shown in FIG. 51. FIG. 51 shows that the band observed in sample 12 and ascribed to the cedar allergen Cj-1 was disappeared in Sample 13. Accordingly, it was shown that most of the cedar allergen Cj-1 protein was denatured by the acid treatment (FIG. 51).

### Example 58

### Inactivation of Cedar Antigen Crj-2 with Acid

(1) Sample 14 (Control)

Added in a 1.5 mL volume disposable tube were 10 μL of the Cj-2 solution and 20 μL of water. NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 14.

(2) Sample 15 (Treatment of Cedar Allergen with HCl)

Added in a 1.5 mL volume disposable tube were 10 μL of the Cj-2 solution and 10 μL of 5 N HCL solution, followed by incubating at 60° C. for 60 minutes. Incubation was stopped by neutralizing the reaction solution by adding 10 μL of 5 N sodium hydroxide solution.

NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 15.

(3) Electrophoresis

The electrophoresis samples 14 and 15 prepared by the methods in (1) and (2), respectively, were analyzed by electrophoresis. NuPage™ MOPS buffer kit for Bis-tris gel was used as an electrophoresis buffer solution, and 15 μL each of samples 14 and 15 prepared above were added in each lane. The protein was stained after electrophoresis using simply Blue™ SafeStain manufactured by Invitrogen Co. The gel was decolorized according to the prescription of the manufacturer.

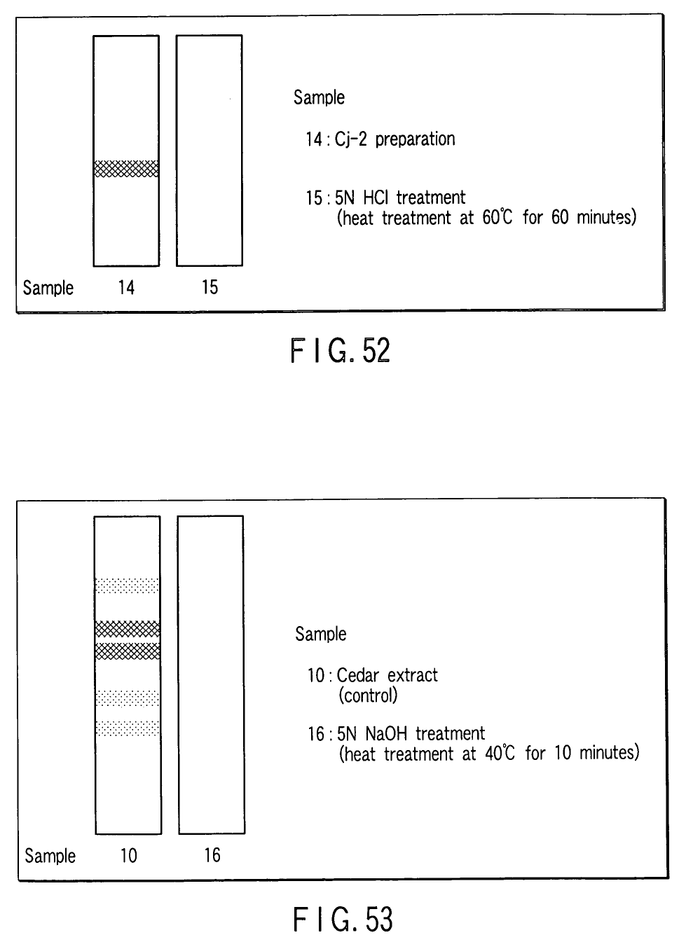

The results are shown in FIG. 52. FIG. 52 shows that the band observed in sample 14 and ascribed to the cedar allergen Cj-2 was disappeared in Sample 15. Accordingly, it was shown that most of the cedar allergen Cj-2 protein was denatured by the acid treatment (FIG. 51).

### Example 59

### Inactivation of Cedar Extract with Alkali

(1) Sample 10 (Control)

Added in a 1.5 mL volume disposable tube were 10 μL of the cedar preparation and 20 μL of water. NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 10.

(2) Sample 16 (Treatment of Cedar Extract with NaOH)

Added in a 1.5 mL volume disposable tube were 10 μL of the cedar preparation and 10 μL of 5 N sodium hydroxide solution, followed by incubating at 40° C. for 10 minutes. Incubation was stopped by neutralizing the reaction solution by adding 10 μL of 5 N hydrochloric acid solution.

NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 16.

(3) Electrophoresis

The electrophoresis samples 10 and 16 prepared by the methods in (1) and (2), respectively, were analyzed by electrophoresis. NuPage™ MOPS buffer kit for Bis-tris gel was used as an electrophoresis buffer solution, and 15 μL each of samples 10 and 16 prepared above were added in each lane. The protein was stained after electrophoresis using simply Blue™ SafeStain manufactured by Invitrogen Co. The gel was decolorized according to the prescription of the manufacturer.

The results are shown in FIG. 52. FIG. 52 shows that the band observed in sample 10 and ascribed to the cedar pollen allergen was disappeared in Sample 16. Accordingly, it was shown that most of the protein derived from the cedar was denatured by the alkali treatment (FIG. 52).

### Example 60

### Inactivation of Cedar Antigen Crj-1 with Alkali

(1) Sample 12 (Control)

Added in a 1.5 mL volume disposable tube were 10 μL of the Cj-1 solution and 20 μL of water. NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 12.

(2) Sample 17 (Treatment of Cedar Antigen with NaOH)

Added in a 1.5 mL volume disposable tube were 10 μL of the Cj-1 solution and 10 μL of 5 N sodium hydroxide solution, followed by incubating at 40° C. for 10 minutes. Incubation was stopped by neutralizing the reaction solution by adding 10 μL of 5 N hydrochloric acid solution.

NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 17.

(3) Electrophoresis

The electrophoresis samples 12 and 17 prepared by the methods in (1) and (2), respectively, were analyzed by electrophoresis. NuPage™ MOPS buffer kit for Bis-tris gel was used as an electrophoresis buffer solution, and 15 μL each of samples 12 and 17 prepared above were added in each lane. The protein was stained after electrophoresis using simply Blue™ SafeStain manufactured by Invitrogen Co. The gel was decolorized according to the prescription of the manufacturer.

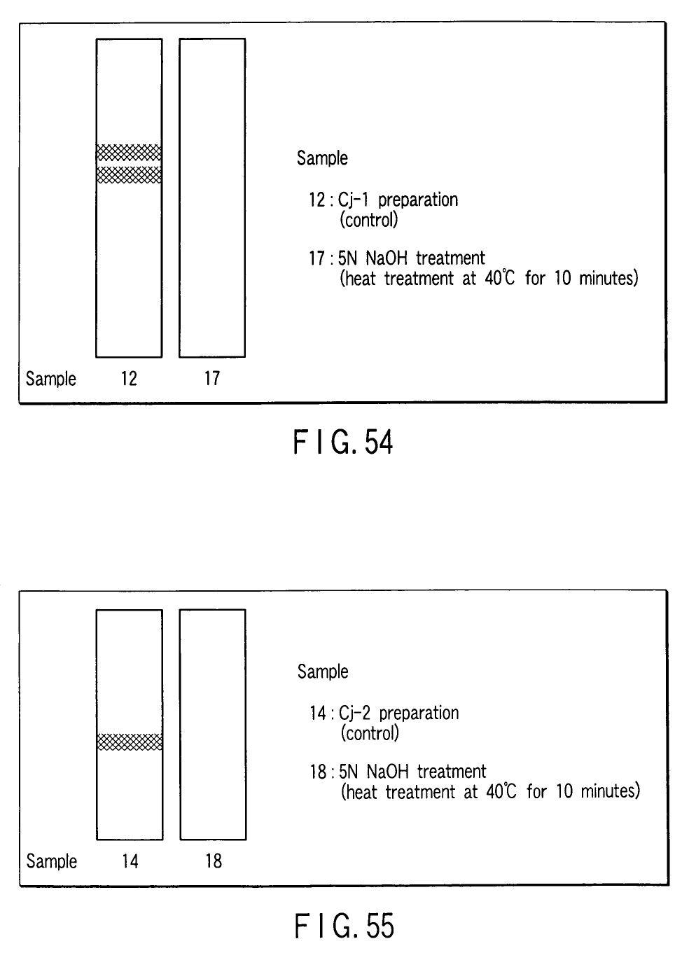

The results are shown in FIG. 54. FIG. 54 shows that the band observed in sample 12 and ascribed to the cedar allergen Cj-1 was disappeared in Sample 17. Accordingly, it was shown that most of the cedar antigen Cj-1 protein was denatured by the alkali treatment (FIG. 54).

### Example 61

### Inactivation of Cedar Antigen Crj-2 with Alkali

(1) Sample 14 (Control)

Added in a 1.5 mL volume disposable tube were 10 μL of the Cj-2 solution and 20 μL of water. NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 14.

(2) Sample 18 (Treatment of Cedar Antigen with NaOH)

Added in a 1.5 mL volume disposable tube were 10 μL of the Cj-2 solution and 10 μL of 5 N sodium hydroxide solution, followed by incubating at 40° C. for 10 minutes. Incubation was stopped by neutralizing the reaction solution by adding 10 μL of 5 N hydrochloric acid solution.

NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 18.

(3) Electrophoresis

The electrophoresis samples 14 and 18 prepared by the methods in (1) and (2), respectively, were analyzed by electrophoresis. NuPage™ MOPS buffer kit for Bis-tris gel was used as an electrophoresis buffer solution, and 15 μL each of samples 14 and 18 prepared above were added in each lane. The protein was stained after electrophoresis using simply Blue™ SafeStain manufactured by Invitrogen Co. The gel was decolorized according to the prescription of the manufacturer.

The results are shown in FIG. 55. FIG. 55 shows that the band observed in sample 14 and ascribed to the cedar allergen Cj-2 was disappeared in Sample 18. Accordingly, it was shown that most of the cedar antigen Cj-2 protein was denatured by the alkali treatment (FIG. 55).

### Example 62

### Electrophoresis and Analysis by Western Blotting of Inactivation of Allergen with Acid and Alkali (1)

(1) Sample 10 (Control)

Added in a 1.5 mL volume disposable tube were 10 μL of the cedar preparation and 20 μL of water. NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 10.

(2) Sample 11 (Treatment of Cedar Extract with HCl)

Added in a 1.5 mL volume disposable tube were 10 μL of the cedar preparation and 10 μL of 5 N HCl solution, followed by incubating at 60° C. for 60 minutes. Incubation was stopped by neutralizing the reaction solution by adding 10 μL of 5 N sodium hydroxide solution.

NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 11.

(3) Sample 16 (Treatment of Cedar Extract with NaOH)

Added in a 1.5 mL volume disposable tube were 10 μL of the cedar preparation and 10 μL of 5 N sodium hydroxide solution, followed by incubating at 40° C. for 10 minutes. Incubation was stopped by neutralizing the reaction solution by adding 10 μL of 5 N HCl solution.

NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 16.

(4) Electrophoresis

The electrophoresis samples 10, 11 and 16 prepared by the methods in (1), (2) and (3), respectively, were analyzed by electrophoresis. NuPage™ MOPS buffer kit for Bis-tris gel was used as an electrophoresis buffer solution, and 15 μL each of samples 10, 11 and 16 prepared above were added in each lane. The protein was stained after electrophoresis using simply Blue™ SafeStain manufactured by Invitrogen Co. The gel was decolorized according to the prescription of the manufacturer.

(5) Western Blotting

After the electrophoresis in (4) above, the proteins were electrically transferred on a pre-treated PVDF membrane. XcellII™ Blot Module manufactured by Invitrogen Co. was used for pre-treatment and transfer of the PVDF membrane according to the prescription of the manufacturer.

Cj-1 antibody as a primary antibody was used by diluting 5,000 times for detecting the allergen by Western blotting in this example. WesternBreeze Chemiluminescent Detection System, Anti Rabbit (registered trademark, manufactured by Invitrogen Co.) was used as a secondary antibody, and blocking and detection reagent according to the prescription of the manufacturer. A luminescent reagent was added to the PVDF membrane after treating as described above, and the membrane was photographed on a Polaroid film using an instant ECL mini-camera (registered trademark) to detect the protein.

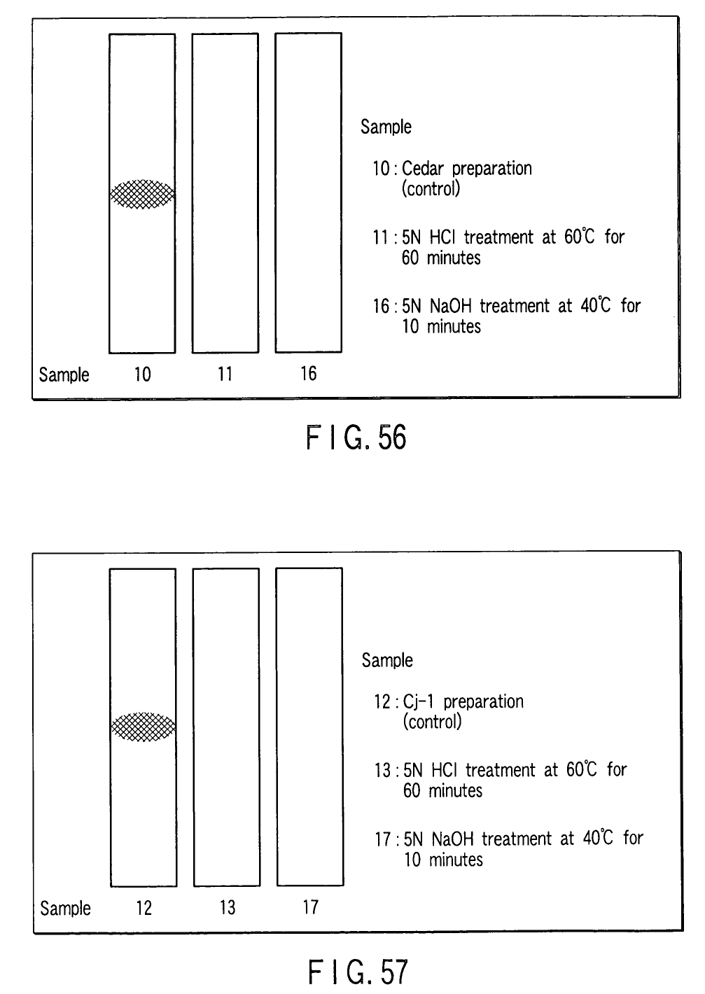

The results are shown in FIG. 56. FIG. 56 shows that the band observed in sample 10 was disappeared in Sample 16. Accordingly, it was shown that most of the cedar antigen Cj-1 protein was denatured by treatment with either an acid or an alkali (FIG. 56).

### Example 62

### Electrophoresis and Analysis by Western Blotting of Inactivation of Allergen with Acid and Alkali (2)

(1) Sample 12 (Control)

Added in a 1.5 mL volume disposable tube were 10 μL of the Cj-1 solution and 20 μL of water. NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 12.

(2) Sample 13 (Treatment of Cedar Antigen with HCl)

Added in a 1.5 mL volume disposable tube were 10 μL of the Cj-1 solution and 10 μL of 5 N HCl solution, followed by incubating at 60° C. for 60 minutes. Incubation was stopped by neutralizing the reaction solution by adding 10 μL of 5 N sodium hydroxide solution.

NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 13.

(3) Sample 17 (Treatment of Cedar Antigen with NaOH)

Added in a 1.5 mL volume disposable tube were 10 μL of the Cj-1 solution and 10 μL of 5 N sodium hydroxide solution, followed by incubating at 40° C. for 10 minutes. Incubation was stopped by neutralizing the reaction solution by adding 10 μL of 5 N HCl solution.

NuPage™ LDS sample buffer, NuPage™ sample reducing agent, and NuPage™ sample antioxidant, each manufactured by Invitrogen Co, were appropriately added according to the prescription of the manufacturer to prepare electrophoresis sample 17.

(4) Electrophoresis

The electrophoresis samples 12, 13 and 17 prepared by the methods in (1), (2) and (3), respectively, were analyzed by electrophoresis. NuPage™ MOPS buffer kit for Bis-tris gel was used as an electrophoresis buffer solution, and 15 μL each of samples 12, 13 and 17 prepared above were added in each lane. The protein was stained after electrophoresis using simply Blue™ SafeStain manufactured by Invitrogen Co. The gel was decolorized according to the prescription of the manufacturer.

(5) Western Blotting

After the electrophoresis in (4) above, the proteins were electrically transferred on a pre-treated PVDF membrane. XcellII™ Blot Module manufactured by Invitrogen Co. was used for pre-treatment and transfer of the PVDF membrane according to the prescription of the manufacturer.

Cj-1 antibody as a primary antibody was used by diluting 5,000 times for detecting the allergen by Western blotting in this example. WesternBreeze Chemiluminescent Detection System, Anti Rabbit (registered trademark, manufactured by Invitrogen Co.) was used as a secondary antibody, and blocking and detection reagent according to the prescription of the manufacturer. A luminescent reagent was added to the PVDF membrane after treating as described above, and the membrane was photographed on a Polaroid film using an instant ECL mini-camera (registered trademark) to detect the protein.

The results are shown in FIG. 57. FIG. 57 shows that the band observed in sample 12 was disappeared in samples 13 and 17. Accordingly, it was shown that most of the cedar antigen Cj-1 protein was denatured by treatment with either an acid or an alkali (FIG. 57).

The invention provides a method for specifically excluding the allergen.

The inventors have reported the inactivating method measuring method of the thick allergen (see Japanese Patent Application No. 2002-106834). The patent reports that allergens derived from living bodies as allergy causing substances could be inactivated irrespective of their species, and activities thereof could be measured. The invention provided an apparatus by which living environments could be largely improved by combining this invention with the art disclosed in Japanese Patent Application No. 2001-224928.

Basic researches for confirming the effect of the virus inactivating agent were carried out in this example using viruses and bacteria capable of manipulating in the laboratory. Inactivation tests of the viruses, bacteria and fungi also carried out using a filter comprising the inactivating agent.

[Inactivating Agent and Inactivating Method]

(1) Material

Bacteriophage that is a virus utilizing bacteria as hosts was used as a representative of the virus capable of manipulating in the laboratory. An experimental system comprising the bacteriophage and E. coli may be considered to be a model of infection and proliferation of the virus in, for example, human and influenza virus. Inactivation of bacteriophage was confirmed by allowing bacteriophage to contact an enzyme or denaturing agent in the liquid in a test tube in the experiment using the bacteriophage.

λ-Phage and M13 phage were used as the bacteriophage. These phages are most frequently studied in molecular biology, and are frequently used for construction of cDNA libraries and analysis of expression of genes today. λ-Phage is a template phage. The phage has two life cycles of killing bacteria cells by proliferation upon infection (bacteriolytic infection), and being transformed into a prophage to survive together with the host bacteria (lysogeny). M13 phage is a single strand DNA phage having a fibrous DNA. The phage is discharged out of the cells without killing the host after proliferation in the host cells. The two phages utilize E. coli XL1-Blue strain and JM109 strain, respectively. Both phages do not infect the bacteria strains that do not have similar properties to respective hosts. Applicability to wide ranges of viruses may be evaluated by using the phages having different properties and hosts in the test. E. coli was cultivated in an LB culture medium (Table 1 below) by shaking cultivation at 37° C. overnight in a conical flask.

TABLE 1
Composition of LB medium

| | | |
|---|---|---|
| Yeast extract | 5 | g |
| NaCl | 5 | g |
| Tripton | 10 | g |
| Water | 1000 | mL |
| Agar (solid medium only) | 15 | g |

(2) Method

### Example 64

### Preparation of λ-Phage Solution

λZAP II vector kit and Gigapack III Packaging kit (registered trademarks, manufactured by Toyobo Co.) were used, and λ-phage was prepared by packaging λ-phage DNA according to the attached manual.

E. coli XL1-blue MRF′ was used as the host cell. Previously cultivated XL1-blue MRF′ was infected with λ-phage, and cultivation was continued. The cultivation medium was centrifuged thereafter to obtain a λ-phage preparation. A solution (10 μL) prepared by appropriately diluting the λ-phage preparation was mixed with 100 μL of XL1-Blue MRF′ (after washing with a SM buffer solution, indicated below in Table 2, to OD of about 1), and the solution was incubated at 37° C. for 15 minutes using the LB culture medium. A LB culture medium (4 mL; preserved in a constant temperature bath at 45° C. after sterilization in a autoclave, named as “top agar hereinafter”) containing 0.7% agarose (manufactured by Takara Bio Co.) was added to this culture medium with stirring, and the mixed solution was poured on a previously prepared solid LB medium containing 1.5% of agar. The culture medium was incubated at 37° C. overnight after confirming that top agar was sufficiently cooled and solidified.

After confirming that plaques had appeared on the solid medium after incubation, 5 mL of the SM buffer solution was added, and the culture medium was allowed to stand in a refrigerator for one day. The solid culture medium was taken out of the refrigerator, the SM buffer solution was withdrawn, and the solution was centrifuged (6000 g×20 minutes, 2° C.). The supernatant obtained was used as a λ-phage solution for the inactivation test hereinafter. The λ-phage solution (0.5 ml each) was dispensed in an Eppendorf tube with a volume of 1.5 mL (named as 1.5 mL volume tube hereinafter), and stored at 4° C. by adding one drop of chloroform.

TABLE 2
SM buffer solution

| Tris-HCl (pH 7.5) | 50 mM |
|---|---|
| NaCl | 100 mM |
| MgSO₄ · 7H₂O | 10 mM |
| Gelatin | 0.01% |

### Sterilized with Autoclave

### Example 65

### Activity Test of λ-Phage

The λ-phage activity test was performed for proving that the experimental system of λ-phage used in the tests hereinafter had been established, and recovery ratios were calculated.

A mixed solution (360 mL) of a physiological saline phosphate buffer solution (PBS) and 10 mM MgSO₄.7H₂O solution were added to 40 μL of the λ-phage solution prepared in Example 64. PBS was used for suppressing possible pH changes during the test, and 10 mM MgSO₄ solution was added together for minimizing the decrease of infection ability of λ-phage not ascribed to the inactivating agent.

The solution was injected into the 1.5 mL volume tube, and 600 μL of a PEG solution (20% PEG6000, 2.5 M NaCl) was added thereafter to allow the tube to stand in ice for 3 hours. The solution was centrifuged at 15,000 rpm for 20 minutes thereafter, and the supernatant was discarded. The SM buffer solution (200 μL) was added to the precipitate obtained (a composite of the λ-phage and PEG), and the precipitate was thoroughly suspended. The λ-phage solution after treating with the PEG solution was used as a PEG precipitation treatment solution.

A solution (10 μL) prepared by diluting the PEG precipitation treatment solution with the SM buffer solution, and a SM suspension solution of XL1-Blue MRF′ (100 μL) were mixed, and the mixed solution was incubated at 37° C. for 15 minutes. After the incubation, 3 mL of top-agar was added to the solution with stirring, and the mixed solution was poured onto the previously prepared solid LB culture medium containing 1.5% of agar. After incubation at 37° C. for 5 hours, the number of the plaques appeared was counted, and pfu (plaque forming unit, pfu/ml) was determined from the number of the plaques.

As a reference, the λ-phage solution prepared in Example 64 was infected with XL1-Blue MRF′ without applying the PEG precipitation treatment, and pfu was determined by counting the number of the plaques. The results are shown in Table 3 below.

TABLE 3
Activity of λ-phage

| | Infection ability |
|---|---|
| Test condition | (pfu/mL) |
| PEG precipitation treatment | 1.9 × 10⁸ |
| Untreated | 9.2 × 10⁸ |

The PEG precipitation treatment solution has a concentration of 1/5 of the concentration of the λ-phase solution prepared in Example 1 since the solution was suspended in the SM buffer solution in the PEG precipitation treatment. The recovery ratio of the λ-phage after the PEG precipitation treatment was calculated as [(1.9×10⁸×5)/(9.2×10⁸)]×100=103%. It was confirmed from this result that λ-phage was almost completely recovered even after the PEG precipitation treatment. It was also confirmed that using PBS and MgSO₄ did not affect on the stability of λ-phage.

### Example 65

### Inactivation Test of λ-Phage 1

Inactivation of λ-phage was tested using the denaturing agent or enzyme. Urea was used as the denaturing agent, and pfu protease S (manufactured by Takara Bio Co.) was used as the enzyme.

Added in 40 μL of the λ-phage solution prepared in Example 1 was 352 μL of PBS+10 mM MgSO₄.7H₂O solution, followed by adding 8 μL of pfu protease S (final concentration of 2%). This solution was added in the 1.5 mL volume tube to prepare a protease treatment solution.

Added in 40 μL of the λ-phage solution was 360 μL of PBS+10 mM MgSO₄.7H₂O solution containing 9 M urea. This solution was added in the 1.5 mL volume tube to prepare an urea treatment solution.

Added in 40 μL of the λ-phage solution prepared in Example 1 was 352 μL of PBS+10 mM MgSO₄.7H₂O solution containing 9 M urea, followed by adding 8 μL of pfu protease S (final concentration of 2%). This solution was added in the 1.5 mL volume tube to prepare an urea/protease treatment solution.

Added in 40 μL of the λ-phage solution prepared in Example 1 was 360 μL of PBS+10 mM MgSO₄.7H₂O solution as a reference to prepare a non-treated solution.

Each solution was incubated at 37° C. for 1 hour. After the incubation, 600 μL of PEG solution (20% PEG6000, 2.5M NaCl) was added, and the solution was allowed to stand in ice for 3 hours. The solution was centrifuged at 15,000 rpm for 20 minutes, and the supernatant was discarded. The SM buffer solution (200 μL) was added to the precipitate obtained, and the precipitate was thoroughly suspended. This suspension solution was diluted with the SM buffer solution, and 10 μL of the diluted solution and the SM suspension solution (100 μL) of E. coli XL1-Bluw MRF′ were mixed followed by incubating at 37° C. for 15 minutes. After the incubation, 3 mL of top-agar was added to the solution with stirring, and the solution was poured onto the previously prepared solid LB culture medium containing 1.5% of agar. After incubating the medium at 37° C. for 5 hours, the number of the plaques appeared was counted to determine pfu. The results are shown in Table 4 below.

TABLE 4
Infection ability of λ-phage in each test condition

| | Infection ability |
|---|---|
| Test condition | (pfu/mL) |
| Protease treatment | 1.8 × 10⁸ |
| Urea treatment | 0 |
| Urea/protease treatment | 0 |
| Untreated | 1.9 × 10⁸ |
| λ-phage solution | 9.2 × 10⁸ |

It was confirmed from the results that λ-phage was not inactivated in the protease treatment solution. However, λ-phage was inactivated in the urea treatment solution in which 9 M urea was added, irrespective of the presence or absence of the protease. Accordingly, it was shown that λ-phage could be inactivated with high concentration urea.

### Example 67

### Inactivation Test of λ-Phage 2

Inactivation of λ-phage was tested by changing the concentration of added urea and urea treatment time.

Mixed with 40 μL of the λ-phage solution prepared in Example 1 was 360 μL of PBS+10 mM MgSO₄.7H₂O solutions containing 0, 3 and 9 M urea, respectively, and this solution was added in the 1.5 mL volume tube. Each mixed solution, and the λ-phage solution as a reference were incubated at 37° C. for 0, 15 and 60 minutes. After the incubation, 600 μL of PEG solution (20% PEG6000, 2.5M NaCl) was added, and the solution was allowed to stand in ice for 3 hours. The solution was centrifuged at 15,000 rpm for 20 minutes, and the supernatant was discarded. The SM buffer solution (200 μL) was added to the precipitate obtained, and the precipitate was thoroughly suspended. This suspension solution was diluted with the SM buffer solution, and 10 μL of the diluted solution and the SM suspension solution (100 μL) of E. coli XL1-Bluw MRF′ were mixed followed by incubating at 37° C. for 15 minutes. After the incubation, 3 mL of top-agar was added to the solution with stirring, and the solution was poured onto the previously prepared solid LB culture medium containing 1.5% of agar. After incubating the medium at 37° C. for 5 hours, the number of the plaques appeared was counted to determine pfu. The results are shown in Table 5 below.

TABLE 5
Infection ability of λ-phage in each test condition (pfu/ml)

| | |
|---|---|
| Treatment | Test condition |

| time | | | | λ-phage |
|---|---|---|---|---|
| (min) | 9 M urea | 3 M urea | 0 M urea | solution |
| 0 | 4 × 10⁶ | 9.4 × 10⁷ | 1.1 × 10⁸ | 8 × 10⁸ |
| 15 | 0 | 1.5 × 10⁸ | 1.1 × 10⁸ | |
| 60 | 0 | 1.1 × 10⁸ | 1.4 × 10⁸ | |

The λ-phage solution has a λ-phage concentration 5 times as large as the concentrations of other treatment solutions. It was confirmed from the results that λ-phage was not inactivated at all in the solution containing no urea, since the plaques with a number of 1/5 as small as the number of the plaques of the λ-page solution was observed. It was also confirmed that λ-phage was not substantially inactivated irrespective of the treatment time when 3 M urea was added. However, λ-phage was completely inactivated at a treatment time of 15 minutes or more when 9 M urea was added. λ-Phage was confirmed to be inactivated to some extent even immediately after adding urea. Accordingly, it was suggested that λ-phage could be quite promptly inactivated by adding 9 M urea.

### Example 68

### Inactivation Test of λ-Phage 3

It was suggested in Example 67 that high concentration urea was effective for inactivating λ-phage. Accordingly, inactivation of λ-phage was further tested by changing the treatment time with 9 M urea.

Mixed with 40 μL of the λ-phage solution prepared in Example 64 was 360 μL of PBS+10 mM MgSO₄.7H₂O solutions containing 9 M urea, and this solution was added in the 1.5 mL volume tube. This mixed solution was incubated at 37° C. for 0, 7.5, 15, 30 and 60 minutes. After the incubation, 600 μL of PEG solution (20% PEG6000, 2.5M NaCl) was added, and the solution was allowed to stand in ice for 3 hours. The solution was centrifuged at 15,000 rpm for 20 minutes, and the supernatant was discarded.

The SM buffer solution (200 μL) was added to the precipitate obtained, and the precipitate was thoroughly suspended.

This suspension solution was diluted with the SM buffer solution of E. coli XL1-Blue MRF′, and 10 μL of the diluted solution and the SM suspension solution (100 μL) of E. coli XL1-Bluw MRF′ were mixed followed by incubating at 37° C. for 15 minutes. After the incubation, 3 mL of top-agar was added to the solution with stirring, and the solution was poured onto the previously prepared solid LB culture medium containing 1.5% of agar. After incubating the medium at 37° C. for 5 hours, the number of the plaques appeared was counted to determine pfu. As a reference, pfu of the untreated solution was determined by the same procedure. The results are shown in Table 6 below.

TABLE 6
Infection ability of λ-phage by adding 9 M urea

| Treatment time | Infection |
|---|---|
| (min) | ability (pfu/mL) |
| 0 | 8.4 × 10⁵ |
| 7.5 | 0 |
| 15 | 0 |
| 30 | 0 |
| 60 | 0 |
| λ-phage solution | 8.4 × 10⁵ |

It was confirmed from the results that λ-phage was inactivated immediately after contacting 9 M urea. The inactivation ratio thereof reached about 99.96%. The presence of 9 M urea seems not to affect the recovery ratio of λ-phage, since λ-phage could be recovered immediately after adding urea.

The inactivation ratio of λ-phage was calculated from the results in Tables 5 and 6 above, and the results are shown in the graph in FIG. 58.
Inactivation ratio=[(1−pfu of each solution)/(pfu of untreated solution/5)]×100

### Example 69

### Preparation of M13 Phage Solution

M13 mp 18RFI (manufactured by Toyobo Co.) was used as M13 phage DNA. E. coli JM109 was used as the host cell. A M13 phage DNA solution (1 μL) and 9 μL of sterilized water were mixed, the cells were transformed to competent cells (E. coli JM109) according to the attached manual. The transformed E. coli was inoculated to a separately prepared LB medium (10 mL), which was cultivated by gently shaking at 37° C. overnight.

The cultivation medium was centrifuged (6,000 g×20 minutes, 4° C.) thereafter to obtain a supernatant. This solution is named as M13 phage preparation. This preparation (10 μL) was added to an SM suspension solution (washed to OD of about 1) of a separately cultivated JM109, and was incubated at 37° C. for 15 minutes. Top-agar (4 mL) was added to the cultivation solution with stirring, the solution was poured onto the previously prepared solid LB culture medium containing 1.5% of agar. The culture medium was incubated at 37° C. overnight after confirming that top agar was sufficiently cooled and solidified.

After confirming that plaques had appeared on the solid medium after incubation, 5 mL of the SM buffer solution was added, and the culture medium was allowed to stand in a refrigerator for one day. The solid culture medium was taken out of the refrigerator, the SM buffer solution was withdrawn, and the solution was centrifuged (6000 g×20 minutes, 2° C.). The supernatant obtained was used as a M13 phage solution for the inactivation test hereinafter. The λ-phage solution (0.5 ml each) was dispensed in an Eppendorf tube with a volume of 1.5 mL (named as 1.5 mL volume tube hereinafter), and stored at 4° C. by adding one drop of chloroform.

### Example 70

### Activity Test of M13 Phage

The M13 phage activity test used in the examples below was performed for proving that the experimental system of M13 phage had been established, and recovery ratios were calculated.

Added in 40 μL of the M13 phage solution prepared in Example 69 was a mixed solution (360 μL) of a physiological saline phosphate buffer solution (PBS) and 10 mM MgSO₄.7H₂O solution. PBS was used for suppressing possible pH changes during the test, and 10 mM MgSO₄.7H₂O solution was added together for minimizing the decrease of infection ability of M13 phage not ascribed to the inactivating agent.

The solution was injected into the 1.5 mL volume tube, and 600 μL of a PEG solution (2% PEG6000, 2.5 M NaCl) was added thereafter to allow the tube to stand in ice for 3 hours. The solution was centrifuged at 15,000 rpm for 20 minutes thereafter, and the supernatant was discarded. An SM suspension solution (200 μL) was added to the precipitate obtained, which was thoroughly suspended in the SM buffer solution. The M13 phage solution after treating with the PEG solution was used as a PEG precipitation treatment solution.

A solution (10 μL) prepared by diluting the PEG precipitation treatment solution with the SM buffer solution, and a SM suspension solution of JM109 (100 μL) were mixed, and the mixed solution was incubated at 37° C. for 15 minutes. After the incubation, 3 mL of top-agar was added to the solution with stirring, and the mixed solution was poured onto the previously prepared solid LB culture medium containing 1.5% of agar. After incubation at 37° C. for 5 hours, the number of the plaques appeared was counted, and pfu (plaque forming unit, pfu/ml) was determined from the number of the plaques obtained.

As a reference, the M13 phage solution prepared in Example 69 was infected with JM109 without applying the PEG precipitation treatment, and pfu was determined by counting the number of the plaques. The results are shown in Table 7 below.

TABLE 7
Activity of M13 phage

| | Infection ability |
|---|---|
| Test condition | (pfu/mL) |
| PEG precipitation treatment | 2.0 × 10¹⁰ |
| Untreated | 1.1 × 10¹¹ |

The PEG precipitation treatment solution has a concentration of 1/5 of the concentration of the M13 phase solution prepared in Example 69 since the solution was suspended in the SM buffer solution in the PEG precipitation treatment. The recovery ratio of the M13 phage after the PEG precipitation treatment was 91% from TABLE 7. It was confirmed from this result that M13 phage was almost completely recovered even after the PEG precipitation treatment. It was also confirmed that using PBS and MgSO₄ did not affect on the stability of M13 phage.

### Example 71

### Inactivation Test of M13 Phage

Inactivation of M13 phage was tested using the denaturing agent or enzyme. Urea was used as the denaturing agent, and pfu protease S (manufactured by Takara Bio Co.) was used as the enzyme.

Added in 40 μL of the M13 phage solution prepared in Example 69 was 352 μL of PBS+10 mM MgSO₄.7H₂O solution, followed by adding 8 μL of pfu protease S (final concentration of 2%). This solution was added in the 1.5 mL volume tube to prepare a protease treatment solution.

Added in 40 μL of the M13 phage solution was 360 μL of PBS+10 mM MgSO₄.7H₂O solution containing 9 M urea. This solution was added in the 1.5 mL volume tube to prepare an urea treatment solution.

Added in 40 μL of the M13 phage solution prepared in Example 1 was 352 μL of PBS+10 mM MgSO₄.7H₂O solution containing 9 M urea, followed by adding 8 μL of pfu protease S (final concentration of 2%). This solution was added in the 1.5 mL volume tube to prepare an urea/protease treatment solution.

Added in 40 μL of the M13 phage solution prepared in Example 69 was 360 μL of PBS+10 mM MgSO₄.7H₂O solution as a reference to prepare a non-treated solution.

Each solution was incubated at 37° C. for 1 hour. After the incubation, 600 μL of PEG solution (20% PEG6000, 2.5M NaCl) was added, and the solution was allowed to stand in ice for 3 hours. The solution was centrifuged at 15,000 rpm for 20 minutes, and the supernatant was discarded. The SM buffer solution (200 μL) was added to the precipitate obtained, and the precipitate was thoroughly suspended. This suspension solution was diluted with the SM buffer solution, and 10 μL of the diluted solution and the SM suspension solution (100 μL) of E. coli JM109 were mixed followed by incubating at 37° C. for 15 minutes. After the incubation, 3 mL of top-agar was added to the solution with stirring, and the solution was poured onto the previously prepared solid LB culture medium containing 1.5% of agar. After incubating the medium at 37° C. for 5 hours, the number of the plaques appeared was counted to determine pfu. The results are shown in Table 8 below.

TABLE 8
Infection ability of M13 phage in each test condition

| | Infection ability |
|---|---|
| Test condition | (pfu/mL) |
| Protease treatment | 0 |
| Urea treatment | 1.1 × 10¹⁰ |
| Urea/protease treatment | 0 |
| M13 phage solution | 1.1 × 10¹¹ |

It was confirmed from the results that M13 phage was completely inactivated with the protease at a final concentration of 2%. The inactivation ratio of M13 phage with 9 M urea was about 50%. M13 phage was completely inactivated in a system containing both the protease and urea. These results suggest that the protease is effective for inactivating M13 phage, and using urea together is also effective.

### Example 72

### Inactivation Test of M13 Phage 2

Inactivation of M13 phage was tested by changing the protease concentration.

Mixed with 40 μL of the M13 phage solution prepared in Example 69 was 352 μL of PBS+10 mM MgSO₄.7H₂O solution. Into this solution in the 1.5 mL volume tube, 0, 0.08, 0.8 and 8 μL each of pfu protease (manufactured by Takara Bio Co.) solution was added. The final concentrations of the added enzyme in the solutions were 0. 0.02. 0.2 and 2%, respectively.

Added into 40 μL of M13 phage solution prepared in Example 69 was 352 μL of PBS+10 mM MgSO₄.7H₂O solution containing 9 M urea. Into this solution in the 1.5 mL volume tube, 0, 0.08, 0.8 and 8 μL each of pfu protease (manufactured by Takara Bio Co.) solution was added. The final concentrations of the added enzyme in the solutions were 0. 0.02. 0.2 and 2%, respectively.

Each treatment solution was incubated at 37° C. for 1 hour. After the incubation, 600 μL of PEG solution (20% PEG6000, 2.5M NaCl) was added, and the solution was allowed to stand in ice for 3 hours. The solution was centrifuged at 15,000 rpm for 20 minutes, and the supernatant was discarded. The SM buffer solution (200 μL) was added to the precipitate obtained, and the precipitate was thoroughly suspended. This suspension solution was diluted with the SM buffer solution, and 10 μL of the diluted solution and the SM suspension solution (100 μL) of E. coli JM109 were mixed followed by incubating at 37° C. for 15 minutes. After the incubation, 3 mL of top-agar was added to the solution with stirring, and the solution was poured onto the previously prepared solid LB culture medium containing 1.5% of agar. After incubating the medium at 37° C. for 5 hours, the number of the plaques appeared was counted to determine pfu. The results are shown in Table 9 below.

TABLE 9
Infection ability of M13 phage at each
concentration of urea and protease

| | Test condition | | |
|---|---|---|---|
| Protease | | | M13 phage |
| concentration | 0 M urea | 9 M urea | solution |
| 0 | 1.0 × 10¹⁰ | 6.3 × 10⁹ | 2.2 × 10¹⁰ |
| 0.02 | 3.9 × 10¹⁰ | 0 | |
| 0.2 | 5 × 10¹⁰ | 0 | |
| 2 | 0 | 0 | |

The number of the plaques was decreased as the protease concentration is increased, and it was confirmed that the inactivation of M13 phage was dependent on the enzyme concentration. The inactivation ratio of M13 phage was increased when 9 M urea was added together, and it was evident that a high inactivation ratio was obtained at a lower protease concentration in the presence of 9 M urea.

### Example 73

### Inactivation Test of M13 Phage 3

Inactivation of M13 phage in the presence of the protease and urea was tested in more detail. The concentration of the protease was fixed at 0.2% from the results in Example 72, and inactivation of M13 phage was tested by changing the urea concentration and treatment time.

Mixed with 40 μL of the M13 phage solution prepared in Example 69 were 359 μL of PBS+10 mM MgSO₄.7H₂O solutions containing 0, 3 and 9 M urea. Into this solution in the 1.5 mL volume tube, 0.8 μL of pfu protease S (manufactured by Takara Bio Co.) solution was added (final concentration of 2%).

The treatment solutions were incubated at 37° C. for 0, 7.5, 15 and 60 minutes, respectively. After the incubation, 600 μL of PEG solution (20% PEG, 2.5M NaCl) was added, and the solution was allowed to stand in ice for 3 hours. The solution was centrifuged at 15,000 rpm for 20 minutes, and the supernatant was discarded. The SM buffer solution (200 μL) was added to the precipitate obtained, and the precipitate was thoroughly suspended. This suspension solution was diluted with the SM buffer solution, and 10 μL of the diluted solution and the SM suspension solution (100 μL) of E. coli JM109 were mixed followed by incubating at 37° C. for 15 minutes. After the incubation, 3 mL of top-agar was added to the solution with stirring, and the solution was poured onto the previously prepared solid LB culture medium containing 1.5% of agar. After incubating the medium at 37° C. for 5 hours, the number of the plaques appeared was counted to determine pfu for evaluating the infection ability. The results are shown in Table 10 below.

TABLE 10
Infection ability (pfu/mL) of M13 phage at each urea concentration
by adding 0.2% protease (test condition)

| Treatment | Test condition | | | |
|---|---|---|---|---|
| time | | | | M13 phage |
| (min) | 9 M urea | 3 M urea | 0 M urea | solution |
| 0 | 1 × 10⁸ | 1.1 × 10¹⁰ | 1.7 × 10¹⁰ | 1.1 × 10¹¹ |
| 7.5 | 0 | 0 | 2.0 × 10¹⁰ | |
| 15 | 0 | 0 | 1.6 × 10¹⁰ | |
| 60 | 0 | 0 | 1.1 × 10⁹ | |

It was confirmed that most of M13 phage is inactivated immediately after contacting these active components when the protease and 9 M urea were added together. The inactivation ratio was only 50% at the time when the phage contacts the active components when the concentration of urea is 3 M. However, the inactivation was almost 100% when 7.5 minutes elapses after the start of the treatment. The phage is not substantially inactivated after adding the protease without urea, and a period of about 1 hour was necessary before the inactivation ratio reached 90%.

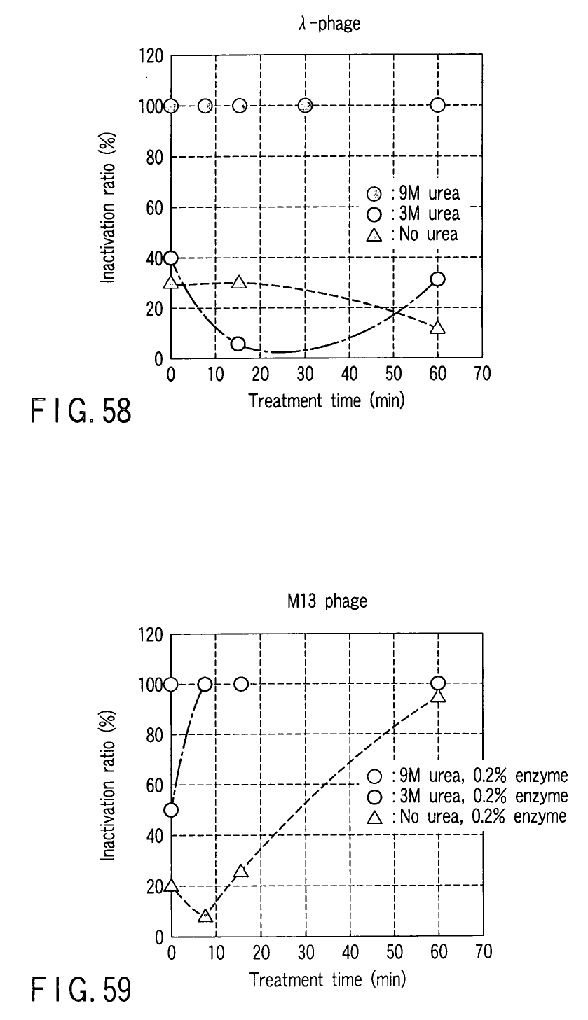

The inactivation ratio of M13 phage was calculated from the results in Table 10, and is shown in the graph in FIG. 59. The inactivation ratio is represented by:
Inactivation ratio=[(1−pfu of each solution)/(pfu of untreated solution/5)]×100

It was shown from the examples above that the virus inactivating agent of the invention is effective for inactivating the virus. The protein denaturing agent and protein decomposing enzyme are effective for inactivating the virus even by using them alone. However, the virus inactivation ratio may be enhanced by using them together, and the invention provides a virus inactivating agent effective for a variety of viruses.

### Example 74

### Disinfection Test Against E. coli 1

The disinfection effect of the inactivating agent of the invention was tested against Escherichia coli JM109 (abbreviated as E. coli hereinafter).

E. coli was cultivated with shaking in the LB culture medium at 37° C. for 10 hours, and was withdrawn by centrifugation (5,000 g×20 minutes, 37° C.) after confirming that the cells were in a logarithmic growth phase. E. coli precipitated was suspended in PBS, and was withdrawn again by centrifugation (5,000 g×20 minutes, 37° C.). The cells were suspended in PBS to prepare an E. coli suspension solution with an OD₆₀₀ of about 15.

Mixed with 32 μL of this E. coli suspension solution were 8 μL of pfu protease S (registered trademark, manufactured by Takara Bio Co.; final concentration of 2%) and 160 μL of PBS solution of 10 M urea. Also prepared were a mixed solution comprising 32 μL of the E. coli suspension solution, 8 μL of the pfu protease S solution and 160 μL of the PBS solution; and a mixed solution comprising 32 μL of the E. coli suspension solution, 8 μL of the PBS solution and 160 μL of the PBS solution containing 10 M urea. A mixed solution comprising 32 μL of the E. coli suspension solution and 168 μL of the PBS solution was also prepared as a reference. Each solution was injected into a 1.5 mL volume tube, and was incubated at 37° C. for 1 hour.

After the incubation, each solution in the tube was centrifuged (10,000 g×20 minutes), and the supernatant was discarded. The precipitate containing the cells was suspended in 400 μL of the PBS solution, and E. coli was cultivated by seeding on an LB solid culture medium. The number of colonies was counted after incubation at 37° C. for 12 hours. The lethal rate of E. coli was calculated by comparing the number of the colonies obtained with the result obtained from E. coli not subjected to the treatment above. The results are sown in Table 11 below.

TABLE 11
Lethal rate of E. coli in each test condition

| Inactivating component | Lethal rate (%) |
|---|---|
| Protease + urea | 100 |
| Protease | 2.3 |
| None | 1.2 |

It was shown that the treatment of this example does not substantially affect E. coli, since the lethal rate of E. coli treated with only the PBS solution as the reference was only about 1%. E. coli was not substantially destroyed by the treatment only with pfu protease S in this example. On the other hand, it was confirmed that E. coli is completely destroyed by a combination of 10 M urea and protease.

### Example 75

### Disinfection Test Against E. coli 2

The disinfection effect of the inactivating agent of the invention was tested by measuring the absorbance of the bacteria suspension solution.

A suspension solution of E. coli with an OD₆₀₀ of about 15 was prepared by the same method as in Example 74. Mixed with 160 μL of this E. coli suspension solution were 40 μL of the pfu protease solution and 800 μL of PBS solution of 10 M urea, and the mixed solution was injected into the 1.5 mL volume tube. Also prepared were a mixed solution comprising 160 μL of the E. coli suspension solution, 40 μL of the pfu protease solution and 800 μL of the PBS solution, and the mixed solution was injected into the 1.5 mL volume tube. A mixed solution comprising 160 μL of the E. coli suspension solution and 840 μL of the PBS solution was also prepared as a reference. Each solution was incubated at 37° C.

A test solution was sampled from each tube at the incubation time of 0, 15, 35 and 60 minutes, and the solution was diluted 1/20 times with PBS to measure the turbidity at 600 nm using a spectrophotometer.

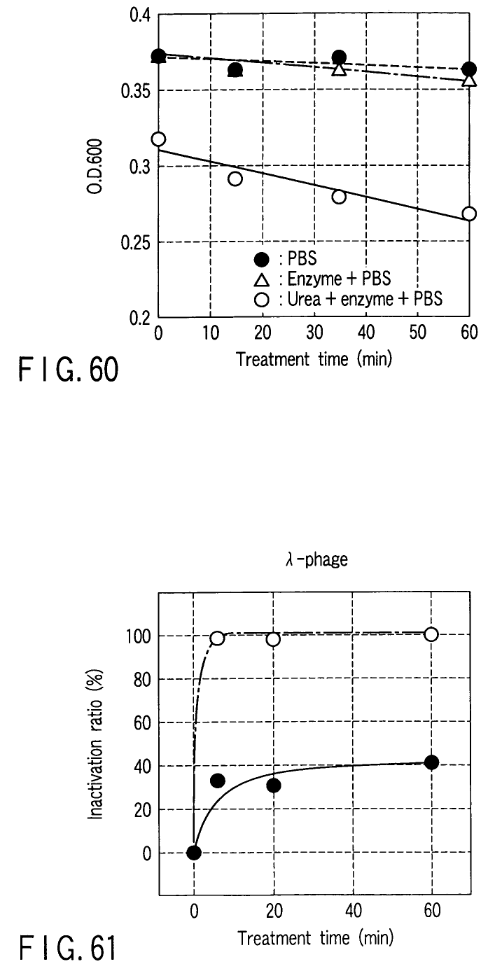

The results obtained are shown in FIG. 60. A decrease of the absorbance shows the decrease of the concentration of the bacteria. The absorbance of the reference was not substantially changed during the experiment period. The absorbance was slightly decreased by treating with the protease only. On the other hand, the absorbance was largely decreased by treating with the protease and 10 M urea. The results suggest that a large disinfection effect against E. coli can be obtained by the inactivating agent containing the protease and urea.

Examples of the filter and air conditioner will be described below.

[Inactivating Filter]

Examples using the virus inactivating filter according to the invention will be described hereinafter.

### Example 76

### Method for Preparing Virus Inactivating Filter

Used as the protein decomposing enzyme was pfu protease S (manufactured by Takara Vio Co.), and an enzyme solution with a protein conversion concentration of 2.1 mg/ml was prepared using PBS. An aqueous urea solution (10 M) was used as a protein denaturing agent. These enzyme solution and urea solution were mixed to prepare an virus inactivating agent.

The composition of the virus inactivating agent adhered on the filter in this example was 1.75 ml of the enzyme solution and 50 ml of the 10 M urea solution, and the proportion of the inactivating agent immobilized on the filter was 1.69. The proportion of the inactivating agent immobilized on the filter described herein was determined from the following equation:
Proportion of immobilized inactivating agent=(dry weight of inactivating agent immobilizing filter)/(dry weight of filter before immobilizing inactivating agent)

The inactivating agent prepared above was adhered on Cellfine N (manufactured by Toyobo Co.), excess agent was removed, and dried at room temperature in a clean bench to adjust to the immobilizing proportion above.

### Example 77

### Inactivation Test Against λ-Phage Using Inactivating Filter 1

The inactivating filter prepared in Example 76 was cut into pieces with a size of 1.5 cm×1.5 cm, and the λ-phage solution (40 μl) prepared in Example 64 was dripped on the filter piece to allow the solution to uniformly permeate in the filter. This filter piece adhering the phage solution was placed in a sterilized 1.5 mL volume tube, and the tube was incubated with a cover at 37° C. The incubation times were 5, 20 and 60 minutes, respectively. After the incubation, the 1.5 mL volume tube containing the filter piece was maintained for 5 minutes in ice.

An SM buffer solution (250 μL) previously cooled at 0° C. was injected into each 1.5 mL volume tube to recover the phage. The phage was recovered by adding 250 μL of the SM buffer solution again for improving the recovery ratio.

The total volume of the recovered phage solution was measured, and a 400 μl of the solution was transferred into a sterilized 1.5 mL volume tube. A PEG solution (600 mL) was added to this tube followed by incubating for 1 hour in ice.

After completing the incubation, the tube was centrifuged at 15,000 rpm for 20 minutes to remove the supernatant. The SM buffer solution (200 μL) was added to the tube containing the remaining precipitate to disperse the precipitate (a composite of the phage and PEG). The infection ability of the phage was evaluated using the dispersion solution of the phage-PEG composite by the same procedure as in the inactivation test above.

The same measurement was performed as a reference using an enzyme immobilized filter “Bio-free” manufactured by Nikki Universal Co. This filter “Bio-free” comprises only the enzyme without providing the protein denaturing agent.

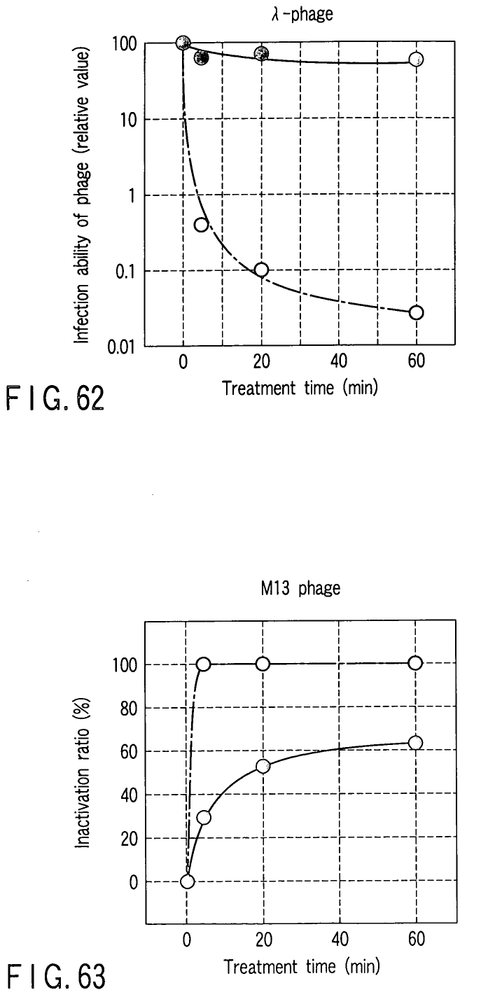

The results obtained are shown in FIGS. 61 and 62. The white circle in the graph shows the results of measurements using the filter according to the invention, while the black circle represents the results of measurements using the reference filter. FIG. 61 shows the inactivation ratio of λ-phage. The untreated inactivation ratio was represented by zero, and the completely inactivated ratio was represented by 100. The inactivation ratio was calculated by: inactivation ratio (%)=100−[(infection ability×(total amount of phage/40/2))/(infection ability of λ-phage solution)]. The infection ability was obtained by the results of measurements in this example. FIG. 62 shows the relative infection ability by taking the infection ability of the original phage solution as 100.

The results obtained showed that λ-phage is almost completely inactivated within a short period of time with the inactivating filter of this example.

### Example 78

### Inactivation Test Against λ-phage Using Inactivating Filter 2

The phage adhered on the filter was withdrawn with the SM buffer solution in Example 78. Since the amount of the recovered phage decreases when the phage is left on the filter, the apparent inactivation ratio of the phage as a result of the measurement is considered to be increased. Accordingly, the phage inactivation ratio due to only the action of the inactivating agent was evaluated in this example.

Specifically, the filter from which the inactivating agent was removed by washing with water was used for the test. Furthermore, the incubation temperature was reduced to 0° C. in the test for suppressing the action of the enzyme contained in the inactivating agent.

As a test under a normal condition, the inactivating filter prepared in Example 76 was cut into pieces with a size of 1.5 cm×1.5 cm, and the λ-phage solution (40 μl) prepared was dripped on the filter piece to allow the solution to uniformly permeate in the filter. This filter piece adhering the phage solution was placed in a sterilized 1.5 mL volume tube, and the tube was incubated at 37° C. for 60 minutes with a cover. The tube was kept in ice for 5 minutes after the incubation.

On the other hand, the filter pieces by cutting the inactivating filter prepared in Example 76 into pieces with a size of 1.5 cm×1.5 cm were washed with distilled water for removing the immobilized inactivating agent. The washed filter was placed at room temperature overnight to thoroughly dry the filter. The phage solution was adhered on the cleaned and dried filter as described above. This filter was placed in a sterilized 1.5 ml volume tube, and was incubated in ice for 60 minutes with a cover.

The SM buffer solution (250 μL each) previously cooled at 0° C. was injected into the tube containing each filter to withdraw the phage. The phage was recovered by adding 250 μL of the SM buffer solution again for improving the recovery ratio.

The total volume of the recovered phage solution was measured, and a 400 μL fraction of the solution was transferred into a sterilized 1.5 mL volume tube. A PEG solution (600 mL) was added to this tube followed by incubating for 1 hour in ice.

After completing the incubation, the tube was centrifuged at 15,000 rpm for 20 minutes to remove the supernatant. The SM buffer solution (200 μL) was added to the tube containing the remaining precipitate to disperse the precipitate (a composite of the phage and PEG). The infection ability of the phage was evaluated by the same procedure as in the inactivation test above using the dispersion solution of the phage-PEG composite.

The same measurement was performed as a reference using an enzyme immobilized filter “Bio-free” manufactured by Nikki Universal Co. This filter “Bio-free” comprises only the enzyme without providing the protein denaturing agent. The results are shown in Table 12.

TABLE 12

| | | 0° C., washed |
|---|---|---|
| Test condition | 37° C. | filter |
| Filter in example | 97.7% (±2.7%) | 15.3% (±9.7%) |
| | n = 5 | n = 4 |
| Reference filter | 42.1% (±10.5%) | 42.5% (±5.2%) |
| | n = 3 | n = 3 |

The enzyme usually has no activity at 0° C. Accordingly, the inactivation ratio obtained at an incubation temperature of 0° C. was not due to the results of the action of the enzyme, rather the inactivation ratio is considered to be the result of the phage remained on the filter and not recovered.

A large difference was observed between the inactivation ratio at 37° C. and inactivation ratio at 0° C. in the reference filter. This result suggests that the inactivation ratio obtained by the reference filter is not ascribed to the enzymatic reaction.

On the other hand, no large difference was observed between the inactivation ratios obtained by incubating at 0° C. and 37° C., respectively. This suggests that the inactivation ratio obtained in the filter of the invention is due to inactivation of the phage by the action of the inactivating agent of the invention.

### Example 79

### Inactivation Test Against M13 Phage Using Inactivating Filter 1

The test was performed by the same method as in Example 14, except that the M13 phage solution prepared in Example 69 was used in place of the λ-phage solution.

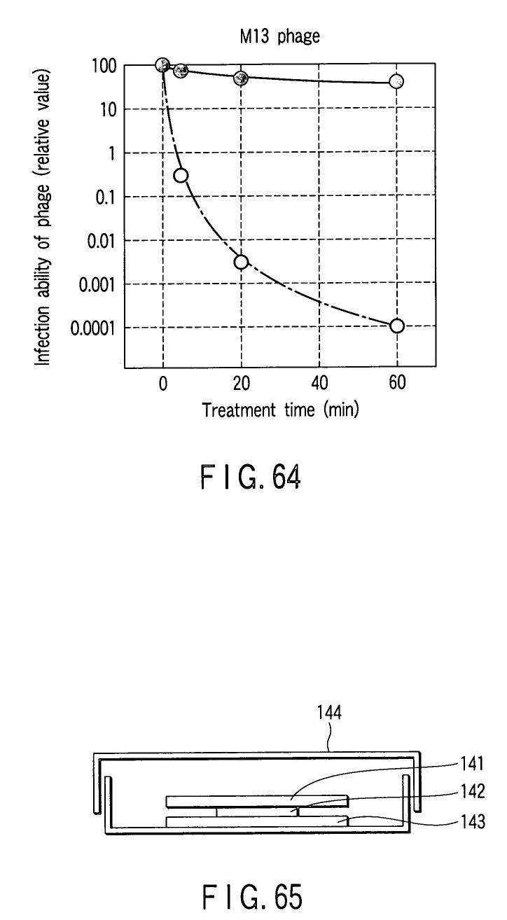

The results obtained were shown in FIGS. 63 and 64. The white circle in the graph shows the results of measurements using the filter according to the invention, while the black circle represents the results of measurements using the reference filter. FIG. 63 shows the inactivation ratio of M13 phage. FIG. 64 shows the relative infection ability of the results of measurements when the infection ability of the original phage was represented by 100.

The results obtained shows that M13 phage is almost completely inactivated by the inactivating filter of the invention within a short period of time.

### Example 80

### Inactivation Test Against M13 Phage Using Inactivating Filter 2

The test was performed by the same method as in Example 77, except that the M13 phage solution prepared in Example 69 was used in place of the λ-phage solution.

The results obtained are shown in Table 13 below.

TABLE 13

| | | 0° C., washed |
|---|---|---|
| Test condition | 37° C. | filter |
| Filter in example | 99.96% (±0.14%) | 50.2% (±16.5%) |
| | n = 13 | n = 6 |
| Reference filter | 62.969% (±23.5%) | 57.4% (±20.2%) |
| | n = 11 | n = 4 |

Generally, the enzyme hardly has activity at 0° C. Accordingly, the inactivation ratio obtained at an incubation temperature of 0° C. is not due to the results of the action of the enzyme, rather the inactivation ratio is considered to be the result of the phage remained on the filter and not recovered.

No large difference was observed between the inactivation ratio at 37° C. and inactivation ratio at 0° C. in the reference filter. This result suggests that the inactivation ratio obtained by the reference filter is not ascribed to the enzymatic reaction.

On the other hand, a large difference was observed between the inactivation ratios obtained by incubating at 0° C. and 37° C., respectively, in the filter in this example. This suggests that the inactivation ratio obtained in the filter of the invention is due to inactivation of the phage by the action of the inactivating agent of the invention.

### Example 81

### Inactivation Test Against Influenza Virus Using Inactivating Filter

Inactivation of the virus by the inactivating filter was tested using influenza virus as a virus having envelop membranes.

The inactivating filter prepared in Example 76 was used by cutting into pieces with a size of 10 mm×10 mm. The virus used was influenza virus A/H1N1 (ATCC No. VR-95).

Influenza virus was inoculated into the allantoic membrane of the embryonated egg, and the egg was cultivated in an incubator at 37° C. The chorioallantoic liquid was harvested and centrifuged (3000 rpm, 30 minutes), and the supernatant was collected. The supernatant obtained was used as a virus preparation.

The virus preparation was adhered on the inactivating filter using an atomizer. The amount of the virus preparation adhered on the filter was 20 μL as converted from the weight difference before and after adhering the preparation onto the filter. The virus preparation was adhered on an untreated filter as a reference that does not carry the inactivating agent by the same method as described above.

Each filter was placed in a desiccator with a relative humidity of 90%, and was incubated at 35° C. for 1 hour. After the incubation, the filter was placed in a disinfected 1.8 mL volume tube, and a 1 ml disinfected PBS solution was added with stirring for 5 minutes to extract the virus. The virus extract solution is named as an evaluation virus solution.

The evaluation virus solution was appropriately diluted. The virus was inoculated in MDCK cells derived from the canine kidney, and the degree of transformation (CPE, five days) was observed using an inverse microscope to calculate TCDI₅₀ (50% infection). As a result, TCDISO of the test using the inactivating filter was 1.77±0.15 (n=3), while TCDI₅₀ of the test using the untreated filter was 4.83±0.06 (n=3).

### Example 82

### Inactivation Test of Polio Virus Using Inactivating Filter

Inactivation of the virus using the inactivating filter was tested using polio virus as a virus having no envelope membranes.

The inactivating filter prepared in Example 76 was used by cutting into pieces with a size of 10 mm×10 mm. Polio virus I Sabin strain (LSc, 2ab) as an weakly toxic polio virus was used.

Polio virus was inoculated to cells (BMG cells) derived from the Vervet monkey kidney, and the cells were cultivated at 37° C. for 4 days. The culture medium was centrifuged (3000 rpm, 30 minutes) to collect the supernatant. The supernatant obtained was named as a virus preparation.

The virus preparation was adhered on the inactivating filter using an atomizer. The amount of the virus preparation adhered on the filter was 20 μL as converted from the weight difference before and after adhering the preparation onto the filter. The virus preparation was adhered on an untreated filter as a reference that does not carry the inactivating agent by the same method as described above.

Each filter was placed in a desiccator with a relative humidity of 90%, and was incubated at 35° C. for 1 hour. After the incubation, the filter was placed in a disinfected 1.8 mL volume tube, and a disinfected PBS solution was added with stirring for 5 minutes to extract the virus. The virus extract solution is named as an evaluation virus solution.

The evaluation virus solution was appropriately diluted. The virus was inoculated in BGM cells derived from the Vervet monkey kidney, and the degree of transformation (CPE, five days) was observed using an inverse microscope to calculate TCDI₅₀ (50% infection). As a result, TCDI₅₀ of the test using the inactivating filter was 3.03±0.15 (n=3), while TCDI₅₀ of the test using the untreated filter was 5.03±0.06 (n=3).

### Example 83

### Disinfection Test of E. coli Using Inactivating Filter

A suspension solution of E. coli with an OD₆₀₀ of about 15 was prepared by the same method as in Example 75. The E. coli suspension solution (40 μL) was uniformly dripped on the inactivating filter prepared in Example 13. The E. coli suspension solution was also dripped on a filter not subjected to the inactivating treatment (untreated filter) as a reference.

Each filter was placed in a 1.5 mL volume tube, and was incubated at 37° C. for 1 hour. After the incubation, each tube was centrifuged by adding 200 μL of PBS. The operation for withdrawing E. coli adhered on the filter was repeated twice.

The solution after withdrawing E. coli was centrifuged (10,000 g×20 minutes), and the supernatant was discarded. The precipitate containing the cells was suspended in 400 μL of PBS, and this suspension solution was seeded on an LB solid culture medium to cultivate E. coli. The culture medium was incubated at 37° C. for 12 hours, and the number of the colonies was counted.

A lethal rate was calculated by comparing the number of the colonies with the number of the colonies obtained from the E. coli suspension solution before the treatment. The lethal rate of the E. coli suspension solution treated with the inactivating filter was 99.1% from the result. On the contrary, the lethal rate of the E. coli suspension solution treated with the untreated filter was 57.5%. A lethal rate of 97.9% was obtained from the comparison between the result obtained by the inactivating filter and the result obtained by the untreated filter.

### Example 84

### Disinfection Test of Fungi Using Inactivating Filter (Dry Test)

The disinfection effect of fungi using the inactivating filter of the invention was tested with reference to the fungus resistance test method (JIS Z 2911:2000) in the JIS standard. Aspergillus niger was used as the fungus.

Five platinum full of the spores of the fungi were suspended in 10 mL of sterilized water containing 0.1% Tween80. This solution was used as a spore suspension solution. A nonwoven fabric (a thickness of 1.5 mm) comprising dry-heat sterilized glass fibers (a diameter of 12 mm) was immersed in the spore suspension solution, and the fabric was dried in a clean bench to prepare a spore carrier.

The spore carrier was placed on the inactivating filter prepared in Example 76 in a sterilized petri dish, and the dish was covered by placing a glass plate (a thickness of 5 mm and an area of 50 mm×50 mm; FIG. 65). The dish was incubated under in a constant temperature/constant humidity environment with a relative humidity of 80% at 35° C. The reference numeral 141 denotes the glass plate, the reference numeral 142 denotes the spore carrier, the reference numeral 143 denotes the filter, and the reference numeral 144 denotes the sterilized petri dish in FIG. 65.

An untreated filter not immobilizing the inactivating agent was also used for the test as a reference.

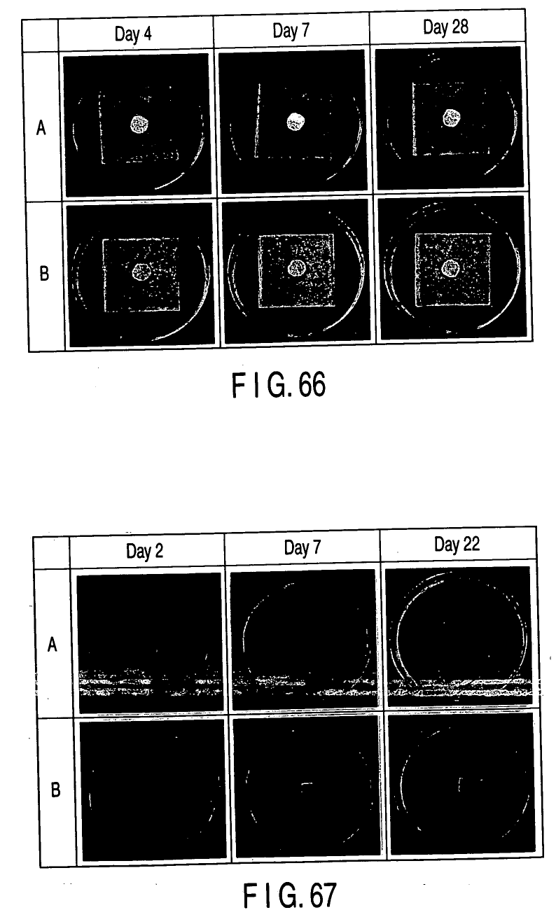

No growth of the fungi was observed on both the inactivating filter and untreated filter as a result of observation of the growth of the fungi (FIG. 66). In FIG. 66, the reference mark A denotes the case when the untreated filter is used, and the reference mark B denotes the case when inactivating treatment filter is used.

Growth of the fungi was observed when the spore suspension solution was cultivated on a potato dextrose agar culture medium concurrently with this test (no data is shown). Therefore, there seems to be no problem on growth of the fungus spore itself used in this test.

### Example 85

### Disinfection Test of Fungi Using Inactivating Filter (Wet Test)

The spore suspension solution was prepared as in Example 84 using Aspergillus niger.

The spore suspension solution (20 μL) was dripped on the inactivating filter (10 mm×10 mm) prepared in Example 76, and the filter was dried in a clean bench. The spore suspension solution was also dripped on the untreated filter and the filter was dried as a reference.

Each filter adhered with the fungus spore was placed on a potato agarose solid culture medium, and the filter was incubated in a constant temperature/constant humidity environment with a temperature of 35° C. and relative humidity of 80%.

While growth of the fungi was confirmed on the untreated filter from the observation of growth of fungi, no growth of the fungi was observed on the activating filter throughout 22 days' cultivation (FIG. 67). In FIG. 67, the reference mark A denotes the case when the untreated filter is used, and the reference mark B denotes the case when inactivating treatment filter is used.

Growth of the fungi was observed when the spore suspension solution was cultivated on a potato dextrose agar culture medium concurrently with this test (no data is shown). Therefore, there seems to be no problem on growth of the fungus spore itself used in this test.

It was shown from the result above that the inactivating filter of the invention has a fungus disinfection effect. Since growth of the fungi was observed on the untreated filter, the filter itself was not considered to have the fungus disinfection ability. Actually, growth of the fungi was suppressed in the presence of the inactivating agent of the invention. While the inactivating agent immobilized on the inactivating filter was worried to be nutrients of the fungi, it was shown that such phenomena does not actually occur.

[Air Conditioner]

One embodiment of the air conditioning indoor unit and air conditioner will be described with reference to the drawings.

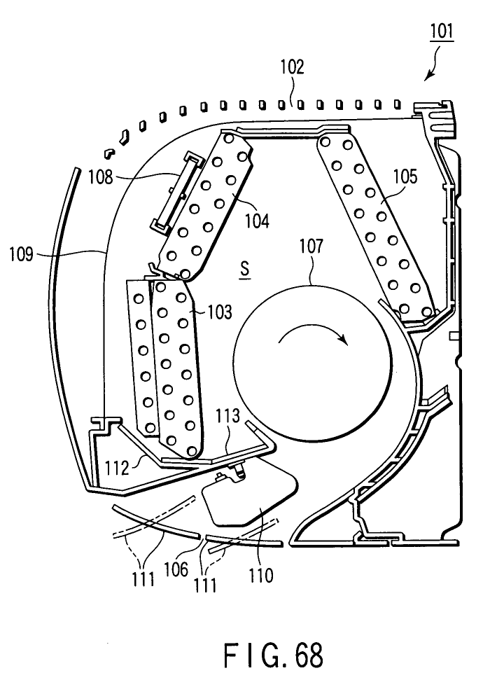

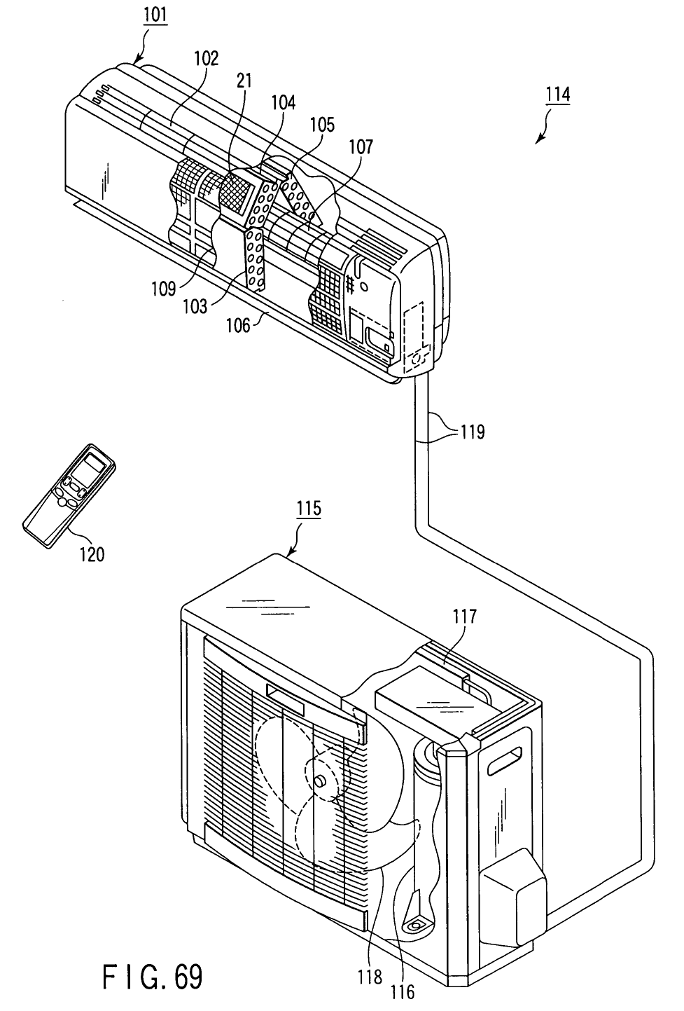

FIG. 68 is a cross section of an air conditioning indoor unit 101, and FIG. 69 is a perspective view showing a schematic configuration of an air conditioner 114 comprising the air conditioning indoor unit 101 and air conditioning outdoor unit 115.

As shown in FIG. 68 or FIG. 69, the air conditioning indoor unit 101 comprises, as principal elements, a suction grill (suction port) 102 for sucking indoor air; indoor heat exchangers 103, 104 and 105 for heating or cooling indoor air sucked from the suction grill 102; a blow-out port 106 for recycling heat-exchange air at the indoor heat exchangers 103 to 105; a cross-flow fan (indoor ventilation means) 107 for sucking air from the suction grill 102 while heat-exchange air from the blow-out port 106 into the room; and a virus inactivating filter 108 disposed at the position above the vicinity at the upstream side of the air flow passageway of the indoor heat exchanger 104. A pre-filter 109 is provided by being disposed from the inner front face to the inner upper face for removing impurities such as dusts from air introduced into the indoor heat exchangers 103 to 105 after flowing through the suction grill 102.

Since the suction grill 102, indoor heat exchangers 103 to 105, blow-out port 106, cross-flow fan 107, and pre-filter 109 in the air conditioning indoor unit 101 are the same as those known in the art, explanations thereof are omitted in the specification of the invention. A blow-out louver 110 and blow out flap 111 known in the art are provided at the blow-out port 106 for controlling the blow direction. The blow-out port 106 is able to open and close by operating a blow-out flap 111.

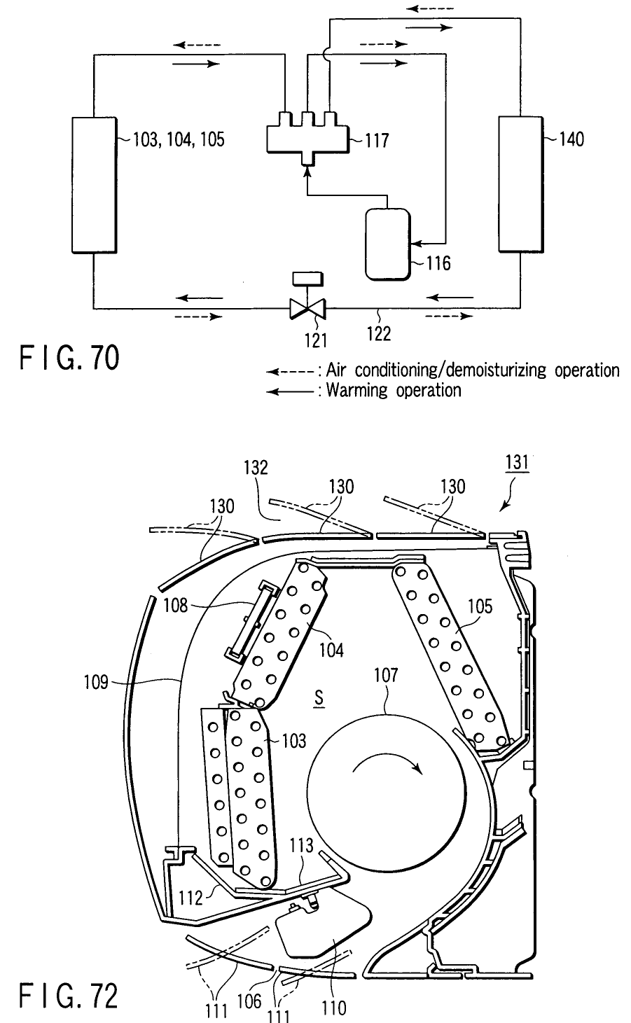

FIG. 69 shows a schematic configuration of the air conditioner 114 comprising the air conditioning indoor unit 101. The reference numeral 115 denotes the air conditioning outdoor unit in FIG. 69. The air conditioning outdoor unit 115 comprises a compressor 116 for compressing the refrigerant, an outdoor heat exchanger 117 for heat-exchange between the refrigerant and outdoor air, and an outdoor fan 118 for heat-exchange between the refrigerator and outdoor air in the outdoor heat exchanger 117. The four-way valve 117 and electronic expansion valve 121 to be described with reference to FIG. 70 hereinafter are also disposed in this air conditioning outdoor unit 115.

The reference numeral 122 denotes a refrigerant piping, whereby the air conditioning indoor unit 101 is connected to the air conditioning outdoor unit 115, and the refrigerator is circulated between the air conditioning indoor unit 101 and the air conditioning outdoor unit 115. The reference numeral 120 denote a remote controller, by which operation conditions of the air conditioner 114 is determined.

The virus inactivating filter (first configuration example) according to the invention will be described below. The filter is graphically the same as those in FIGS. 16a and 16B described in Example 13. However, the “allergen inactivating filter” described in Example 13 is comprehended as the “virus inactivating filter” in this example, and the “enzyme” is recognized as the “virus inactivating agent” (the same in second to seventh configuration examples hereinafter).

The virus inactivating filter comprises the filter main body 22 and the virus inactivating agent (simply called as “inactivating agent” hereinafter) 24 directly immobilized on the fiber 23 constricting the filter main body 22. Examples of the fiber 23 include glass, rayon, cellulose, polypropylene, polyethylene terephthalate, polyacrylic acid and polyacrylamide fibers. Ultra-moisture absorbing fibers such as Cellfine N (manufactured by Toyobo Co.) may be also used.

The inactivating agent may be physically as well as chemically immobilized on the fiber 23. For example, the carboxyl group on the base material is converted into azide groups, and the active components can be immobilized on the base material by chemical bonds between the amide group and inactivating agent to be contained. Functional groups such as hydroxyl groups and amino groups other than the carboxyl groups may be utilized for the chemical bond. The chemical immobilization methods have been known in the art (Sin Jikken Kagaku Koza Seibutu Kagaku (1), p.363-409, Maruzen Co., 1978).

Since the inactivating agent 24 having a function for inactivating the viruses is immobilized on the filter main body 22 according to the virus inactivating filter of the first configuration example, the quantity of the virus capable of being inactivated may be largely increased.

A second configuration example of the virus inactivating filter will be then described. The filter is the same as the filter in FIG. 17 described in Example 14 in the drawing. The inactivating agent 24 is immobilized on the carrier 25 having a water absorbing and/or moisture absorbing property as shown in FIG. 17, and the carrier 25 is fixed on the fiber 26 using a binder (not shown). Examples of the material of the carrier 25 include synthetic materials such as polyacrylic acid, polyacrylamide and polyvinyl alcohol, natural materials such as cotton, wool, sodium alginate, mannan and agar, and regenerated materials such as rayon. Examples of the materials of the fiber 26 include polymer materials such as polyethylene (PE), polypropylene (PP), polyethylene terephthalate (PET) and polyamide (PA).

Since the inactivating agent 24 is immobilized on the water absorbing and/or moisture absorbing carrier 25 according to the second configuration example of the virus inactivating filter, and the carrier 25 is fixed to the fiber 26 using a binder, the agent has the same effect as the first configuration example described above.

A third configuration example of the virus inactivating filter will be described below. The filter is the same as the filter in FIG. 18A described in Example 15 in the drawing. The filter in the third configuration example comprises the carrier 25 immobilizing a plurality of inactivating agents 24, and two base materials 27 and 28 sandwiching the carrier from above and below the carrier.

Examples of the material of the carrier 25 include polyacrylic acid, polyacrylamide, polyvinyl alcohol, cotton, wool, rayon, sodium alginate, mannan and agar. The base materials 27 and 28 are made of the fiber 26 comprising a nonwoven fabric. The base material 28 located under the carrier 25 is preferably made of a nonwoven fabric having a mesh smaller than the diameter of the virus particles (a diameter of several tens to several hundreds microns).

Since the flat virus inactivating flat filer 21 according to the third configuration example comprises the carrier 25 immobilizing the inactivating agent 24 sandwiched between the base materials 27 and 28, the filter has the same effect as the constitution described above.

A fourth configuration example of the virus inactivating filter will be described below. The filter is the same as the filter in FIG. 18B described in Example 15 in the drawing. The filter in the fourth configuration example is an open sandwich type virus inactivating flat filter as shown in FIG. 18B. This filter also exhibits the same effect as in the first configuration example described above.

The virus inactivating filters 21 in the first to fourth configuration examples as described above may be used by disposing in the ventilation passageway of the air conditioning indoor unit 101 or the like by being housed in a case 29 as shown in FIG. 19.

A fifth configuration example of the virus inactivating filter will be described below. The filter is the same as the filter in FIG. 20A described in Example 16 in the drawing. The virus inactivating filter 21 in the fifth configuration example comprises the filter main body 22 made of the fiber directly immobilizing the inactivating agent as shown in FIG. 20A, and the filter is constructed by pleating the filter main body 22.

According to the pleated virus inactivating filter 21 in the fifth configuration example, the filter main body is constructed with fibers directly immobilizing the inactivating agent, and the filter main body 22 is pleated. Accordingly, the filter has a lower pressure loss as compared with the filter having the constitutions described above. In addition, the trapping ratio may be increased due to increases opportunity for making contact with the virus while evaporation of moisture is suppressed.

A sixth configuration example of the virus inactivating filter will be described below. The filter is the same as the filter in FIG. 20B described in Example 17 in the drawing. The virus inactivating filter comprises rod-like members 31 comprising bundles of fibers having a circular cross section and immobilizing the inactivating agent 24 in this sixth configuration example as shown in FIG. 20B. The rod-like member 31 is connected to supporting members 32 and 33 at both ends.

The rod-like member 31 is formed by the fibers immobilizing the inactivating agent 24 in the rod-like virus inactivating filter according to the sixth configuration example, and both ends of the rod-like member 31 are connected to the supporting members 32 and 33. Accordingly, the filter has a lower pressure loss as compared with the filters in the first to fourth configuration examples, and the inactivating ability is large since the quantity of the immobilized inactivating agent is increased while the service life is prolonged.

While the cross-section of the rod-like member was circular in the sixth configuration example, the shape is not particularly restricted, and may be triangle, rectangle, elliptical or hollow shape. The direction of alignment of the rod-like member is not particularly restricted, and the members may be aligned in a vertical or horizontal direction, or may be aligned aslant. Alternatively, the members may be crossed with each other. When the virus inactivating filter in this configuration example is mounted in the air conditioning indoor unit 101, it is preferably attached at a position where ventilation is rapid such as the blow-out port 106, or at both the suction grill 102 and blow-out port 106.

A seventh configuration example of the virus inactivating filter will be described below. The filter is the same as the filter in FIG. 20C described in Example 18 in the drawing. The virus inactivating filter comprises the inactivating agent 24 immobilized on the surface of the porous member 34 such as urethane in the seventh constituting example shown in FIG. 20C.

The sponge-like virus inactivating filter according to the seventh configuration example can exhibit the same effect as in the first to fourth configuration examples.

The materials of the filter main body available include natural fibers such as cotton and wool; regenerated fibers such as rayon and cellulose acetate fibers; nonwoven or woven fabrics of synthetic fibers such as polyethylene, polyethylene terephthalate and polyamide fibers; glass fiber mats; metal fiber mats; synthetic resins such as acrylic, acrylamide and polyvinyl alcohol resins; and water absorbing and/or moisture absorbing materials as natural and regenerated materials of sodium alginate, mannan and agar. The enzyme is immobilized directly or via a carrier on the filter main body comprising these materials.

Since the virus inactivating agent of the invention involves in the reaction in a liquid phase, fine liquid phases may be formed on the surface of the fiber or within the carrier by using a water absorbing and/or moisture absorbing material for the fiber of the filter or for the carrier in order to activate the inactivating agent.

While the inactivating agent is active at room temperature in the constitution of the filter as described above, it is more activated in a high temperature-high humidity atmosphere since the enzyme contained in the virus inactivating agent becomes more active at higher temperatures. The preferable temperature is in the range not exceeding the optimum temperature of the enzyme, and not higher than the durable temperatures of the air conditioner and filter. For example, when pfu protease S (manufactured by Takara Bio Co.) used in the foregoing examples is used, the preferable temperature is in the range of 30 to 80° C.

The air conditioning indoor unit 101 provided with the virus inactivating filter 108 as described above comprises inactivating agent activating means for maintaining the inner space S in an inactivating agent activating atmosphere with high temperature and high humidity. The inner space S means the ventilation passageway (space) from the suction grill 102 to the air blow-out port 106 of sucked air. The inactivating agent activating means in the first embodiment may be operated by effectively utilizing the constitution elements commonly provided in the air conditioner 114 except the virus inactivating element, without adding any special constitution elements.

A virus inactivating operation mode is provided in the air conditioner 114 in order to permit the refrigerant circuit comprising an existing heat exchanger to function as inactivating agent activating means. The inner space S of the air conditioning indoor unit 101 is maintained in a high temperature-high humidity atmosphere for activating the virus inactivating agent by allowing the control means of the air conditioner 114 to execute this operation mode. Viruses previously trapped on the virus inactivating filter 21 is destroyed to irreversibly inactivate the virus in the inactivation treatment process by the action of the activated virus inactivating agent.

Moisture is required in order to maintain a high temperature and high humidity atmosphere in the virus inactivating operation mode. Accordingly, cooling operation of the indoor heat exchangers 103, 104 and 105 provided in the air conditioning indoor unit 101 is continued for a given period of time, and condensed water formed on the surface of the heat exchangers is used for moisturizing. The refrigerant may be circulated in the cooling operation of the heat exchangers 103 to 105 as in the cooling operation and demoisturizing operation using the heat exchanger as an evaporator. This cooling operation is called as “condensed water forming operation” hereinafter.

The refrigerant is circulated by operating the compressor 116 at the air conditioning outdoor unit 115 side and outdoor fan 118 in the condensed water forming operation as shown in the refrigerant circuit diagram in FIG. 70, and the cross-flow fan 107 is operated by opening the flap 111 provided at the blow-out port 106 at the air conditioning indoor unit 101 side.

The refrigerant circulation passageway is formed so that the circulation direction of the refrigerant is selectively switched with the four-way valve 117 after being discharged from the compressor 116 as shown in the solid line arrow in FIG. 70, and the refrigerant returns the compressor 116 after flowing in the order of the outdoor heat exchanger 140, electronic expansion valve 121, indoor heat exchangers 103, 104 and 105 and four-way valve 117 clockwise. The refrigerant as an air-liquid two phase flows in the indoor heat exchangers 103, 104 and 105 for heat exchange between the refrigerant and air when such refrigerant flow passageway is formed as described above. Air after releasing the heat of evaporation is cooled, and moisture in air is condensed and adheres on the surface of the heat exchanger by decreasing the temperature. Condensed water thus formed drips in the drain receiver 112 from the surface of the indoor heat exchangers 103, 104 and 105, and is discharged to the outside of the air conditioning indoor unit 101 through a drain flow passageway (not shown).

The operation mode is transferred to a heating operation mode for making the inner space S high temperature-high humidity by vaporizing condensed water by heating.

The refrigerant discharged from the compressor 116 flows in a counterclockwise direction as an inverse direction to the direction in the condensed water forming operation by switching the four-way valve 117 in the heating operation, as shown by the broken line arrow in the refrigerant flow circuit diagram in FIG. 70. In other words, the refrigerant discharged from the compressor 116 flows out of the four-way valve 117, and returns to the compressor 115 after flowing in the order of the indoor heat exchanger 103, 104 and 105, electronic expansion valve 121, outdoor heat exchanger 140 and four-way valve 117.

The high temperature-high pressure gas refrigerant fed to the indoor heat exchanger 103, 104 and 105 is condensed by heat exchange with air, by allowing the refrigerant to circulate in the heating operation as in the worming operation. Since the indoor heat exchanger exhibits a function for radiating the heat as a condenser, condensed water adhered on the surface of the heat exchanger can be vaporized by using the radiated heat as a heating medium.

Since the compressor 116 and outdoor fan 118 of the air conditioning outdoor unit 115 are operated in the heating operation different from the warming operation in order to accelerate evaporation of condensed water, operation of the cross-flow fan 107 at the air conditioning indoor unit 101 side is stopped and the blow-out port 106 is closed by operating the blow-out flap 111. Consequently, the inner space S of the air conditioning indoor unit 101 becomes semi-hermetic by closing the blow-out port 106. Since the vapor of condensed water vaporized by heat radiation (heating) of the indoor heat exchangers 103, 104 and 105 stays in the inner space S to increase the temperature thereof, the inactivating agent activating atmosphere in a high temperature-high humidity state of the inner space S may be readily achieved.

However, since the vapor formed by vaporization of condensed water flows through a passageway for allowing the vapor to vertically ascend, the virus inactivating filter 21 is placed above the indoor heat exchangers 103, 104 and 105, preferably just above the indoor heat exchanger, in order to permit the virus inactivating filter 21 to reliably absorb moisture.

The virus inactivating filter 21 may be disposed at least in the ventilation passageway in the usual cooling operation and warming operation, and at the site where the filter is able to contact evaporated air formed by the heating operation within the indoor apparatus. The installation position of the inactivating filter is not always restricted at above the indoor heat exchanger.

Since the inactivating agent 24 immobilized on the virus inactivating filter 21 is activated when the inner space S is controlled to be an inactivating agent activating atmosphere, the virus trapped on the filter 21 is inactivated by the action of the inactivating agent 24. The heating operation time for inactivating the virus may be appropriately determined depending on the desired virus inactivating ratio.

Since the inside space S of the air conditioning indoor unit 101 can be maintained in the virus inactivating atmosphere for a required time period by the condensed water forming operation and heating operation, the inactivating agent 24 immobilized on the virus inactivating filter 21 is activated in this atmosphere, and the trapped virus can be efficiently inactivated.

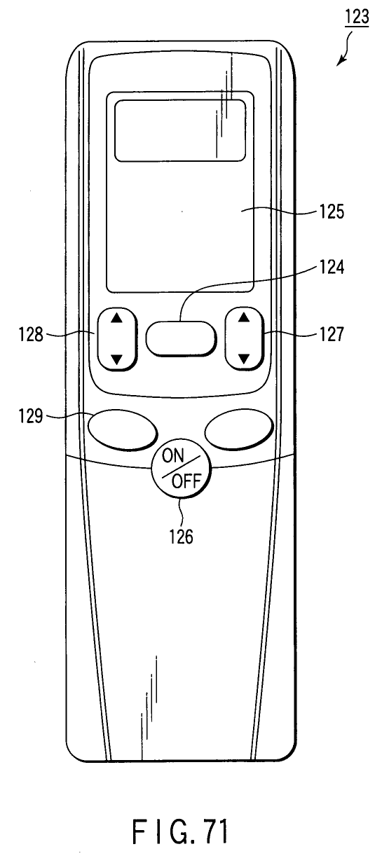

The virus inactivating operation mode can be immediately implemented by operating a switch provided at an appropriate location such as an operation panel. This switching operation may be implemented, for example, by pressing a previously provided virus clear button 124 on a remote controller 123 as shown in FIG. 71. In other word, a specified control signal for executing the virus inactivating operation mode is generated by pressing the virus clear button 124. When the virus clear button 124 of the remote controller 123 is pressed, the control signal such as an infrared light signal is transmitted to a receiver (not shown) of the air conditioning indoor unit 101. The remote controller 123 shown in the drawing also comprises, for example, a display 125, a start/stop operation button 126, a temperature set switch 127, a humidity set switch 129 and an operation mode switch button 129 in addition to the virus clear button 124 described above.

The control signal is sent from the receiver to a controller (not shown) of the air conditioner 114. The controller after receiving the signal inactivates the virus by executing the condensed water forming operation and heating operation based on predetermined control steps. Such virus inactivating operation mode is preferentially executed over other operation modes when the control signal generated by pressing the virus clear button 124 is sent to the controller. In other words, the cooling operation or warming operation under implementation is suspended to switch the operation to the virus inactivating operation mode.

The virus inactivating operation mode may be appropriately interrupted if necessary. The control signal for interrupting the virus inactivating operation mode may be generated either by pressing the virus clear button again, or by providing an exclusive use interruption button on the remote controller 123. Since implementation and interruption of the virus inactivating operation mode is made to be immediately selectable by switching operation of the remote controller 123, the virus can be readily inactivated by a simple operation. The virus inactivating operation mode may be operated in communication with a cooling/warming operation timer function that has been provided in the air conditioner 114.

While a high temperature-high humidity atmosphere is formed in the virus inactivating operation mode, the time required for attaining the desired level varies depending on the indoor and outdoor environment (temperature and humidity). In other words, the time required for obtaining a desired volume of condensed water formed by the condensed water forming operation, and the time required for attaining a desired temperature and humidity are different depending on the environment as described above. Accordingly, it is preferable to control the operation condition so that condensed water is readily formed on the surface of the indoor heat exchangers 103, 104 and 105, when the apparatus is operated for forming condensed water.

Examples of the operation condition by which condensed water is readily formed will be described below. In a first example, the aperture of the electronic expansion valve 35 provided as a restriction mechanism is reduced to be smaller in this operation mode relative to the aperture in a usual cooling operation. As a result, since the surface temperature of the indoor heat exchangers 103, 104 and 105 is more reduced by increasing the amount of heat absorption by the refrigerant, the volume of condensed water dewed on the surface of the heat exchanger can be increased. The aperture of the electronic expansion valve 121 may be adjusted depending on a detected value (room temperature) of room temperature detection means provided in the air conditioning indoor unit 101, and the aperture of the electronic expansion valve 121 is reduced as the room temperature is higher.

In a second example, the ventilation rate flowing through the indoor heat exchangers 103, 104 and 105 may be reduced by reducing the ventilation rate by a low speed operation in which the rotation speed of the cross-flow fan 107 is lowered from the rotation speed in the cooling operation. Since the surface temperature of the indoor heat exchangers 103, 104 and 105 is reduced by reducing the amount of heat absorption of air by this operation, the volume of condensed water dewed on the surface of the heat exchanger can be increased.

In a third example, the rotation speed of the outdoor fan 118 provided in the air conditioning indoor unit may be controlled by detecting the outdoor temperature. Since the volume of the refrigerant condensed in the outdoor heat exchanger 117 increases by setting the rotation speed of the outdoor fan 118 higher as the outdoor temperature is higher, the volume of the refrigerant in the gas-liquid two phase fed to the indoor heat exchangers 103, 104 and 105 are also increased. Accordingly, since the surface temperature of the indoor heat exchangers 103, 104 and 105 are more reduced, the volume of condensed water dewed on the surface of the heat exchanger also increases.

The first to third examples may be employed alone, or plural examples may be employed in combination, or all the examples may be employed together.

However, it is not always restricted to the control for executing the condensed water forming operation in advance to the heating operation. Instead, the usual air conditioning operation may be defined to be a cooling operation when condensed water is formed by the usual air conditioning operation, and the virus inactivating operation mode may be executed by the heating operation after the air conditioning operation.

The original object for inactivating the virus by activating the inactivating agent 24 can be attained by executing the condensed water forming operation and heating operation in the virus inactivating operation mode. However, the efficiency of the virus inactivating operation may be improved and the service life of the inactivating agent 24 may be prolonged by adding the following operations before and after the virus inactivating operation.

A trapping operation executed before the condensed water forming operation will be described at first. This trapping operation means an operation for trapping the virus in the room on the virus inactivating filter 21. Indoor air is sucked through the suction grill 102 by operating the cross-flow fan 107, and is returned in the room from the blow-out pot 106 after allowing air to flow through the virus inactivating filter 21. Since the object of the trapping operation is to trap the virus in air with the virus inactivating filter 21, indoor air may flow through the virus inactivating filter 21, or air may be merely circulated by a ventilating operation. Naturally, since indoor air is circulated through the virus inactivating filter 21 by the usual air conditioning, demoisturizing and warming operation, the operation conditions may be appropriately selected from the ventilation, air conditioning, demoisturizing and warming operations depending on indoor conditions and preference of users.

While circulation of air as described above permits air to flow through the filter main body 22 or the like when air flows through the virus inactivating filter 21, a large proportion of the virus circulating together with air is trapped without passing through the filter. Accordingly, most of the viruses in the room are trapped on the inactivating filter 21 by a continuous trapping operation for a proper period of time considering the size of the room, the anticipated amount of the virus and trapping ability of the virus inactivating filter 21. Since many viruses are inactivated by one virus inactivating operation when the virus inactivating operation mode for trapping most of the virus is executed, the virus in the room is efficiently inactivated to enable the indoor environment containing few viruses to be attained.

It is desirable to promptly improve the high temperature-high humidity environment in the inner space S after completing the heating operation in the virus inactivating operation mode. Since the inactivating agent 24 is hydrolyzed with moisture remaining in the virus inactivating filter 21, it is preferable to resume an environment for activating the inactivating agent 24 in a usual environment, or in a low temperature-low humidity atmosphere, for suppressing autolysis of the inactivating agent considering the service life of the inactivating agent 24. This is also preferable for suppressing time-dependent deterioration of the inactivating agent.

Consequently, the ventilating operation is executed within an appropriate operation time after completing the heating operation after a time lapse of a predetermined inactivating agent activating time for maintaining the inactivating agent in an activated state, when the air conditioning indoor unit is provided with a ventilator (not shown) for exhausting indoor air to open air. High temperature-high humidity air in the inner space S can be exhausted to open air by operating a ventilation fan (not shown) while the inner space S becomes semi-hermetic by closing the blow-out flap 111 in this ventilating operation, in order to prevent air conditioning feeling from being deteriorated due to direct efflux of high temperature-high humidity air into the room.

A ventilating operation using the cross-flow fan 17 as well as the ventilating fan is started after exhausting high temperature-high humidity air to open air by the ventilating operation for a predetermined period of time. The inner space S is maintained to be semi-hermetic by closing the blow-out flap 111. The virus inactivating filter 21 can be dried by demoisturizing by air flow arising from ventilation and air circulation in the inner space S. An appropriate operation time is determined depending on the volume of the inner space S for the deterioration preventing operation by taking advantage of ventilation and air circulation.

The high temperature-high humidity environment of the inner space S is promptly improved and the time period when the inactivating agent 24 is uselessly activated is shortened, when the control means of air conditioner 114 executes the deterioration preventive operation mode for switching to the ventilating operation and air blowing operation after completing the virus inactivating operation mode. Consequently, the service life of the inactivating agent 24 can be prolonged, or the period before exchange of the virus inactivating filter 21 is prolonged, by suppressing the agent from being deteriorated.

While the air conditioning indoor unit is assumed to be provided with the ventilator, the ventilating operation is impossible in the air conditioning indoor unit not provided with the ventilator. Accordingly, an air blow operation is performed with the cross-flow fan 107 in the semi-hermetic inner space S after completing the heating operation, and the virus inactivating filter 21 may be dried by the air flow generated by this operation.

The deterioration preventive operation, in which the air blow operation and ventilating operation have been used together when the ventilator is provided, is executed by using the air blow operation only when no ventilator is provided. However, the deterioration preventive operation for removing moisture from the inactivating agent carrier may be selected from the use of the air blow operation only and concomitant use of the air blow operation and ventilating operation when the ventilator is provided.

Since the suction grill 102 is always open in the air conditioning indoor unit 101 that has been described above, the inner space S becomes semi-hermetic during the heating operation when the blow-out flap 111 is closed.

Accordingly, the air conditioning indoor unit 131 that is operated by closing the inner space S will be described as a modification of the air conditioning indoor unit described above with reference to FIG. 72. Suction port open-close means such as the suction port flap 130 is provided at the suction grill 132, and the suction grill 132 is closed, if necessary, during the heating operation, for example. Consequently, the inner space S is becomes hermetic by closing the suction grill 132 and blow-out port 106 together with the flap during the heating operation, and high temperature-high humidity air for inactivating the virus is hardly leaked to the outside.

The temperature and humidity in the inner space S may be readily maintained with no leakage of air to the outside by executing such heating operation in a hermetic state, and the inactivating agent 24 can be efficiently activated. In other word, a virus inactivating atmosphere may be formed within a shorter period of time than in the heating operation in a semi-hermetic state, and the heating energy consumed for maintaining the high temperature-high humidity atmosphere as well as the volume of condensed water can be reduced.

It is preferable to agitate the inner space S by rotating the cross-flow fan 107 during the heating operation in the hermetic inner space S. Such agitation permits the high temperature-high humidity atmosphere in the hermetic inner space S to be homogeneous.

The inactivating agent 24 is activated in the entire region of the virus inactivating filter 21. Since the inactivating agent 24 functions over the entire region of the virus inactivating filter 21 and can inactivate the virus efficiently, the ability as the filter may be efficiently utilized in maximum.

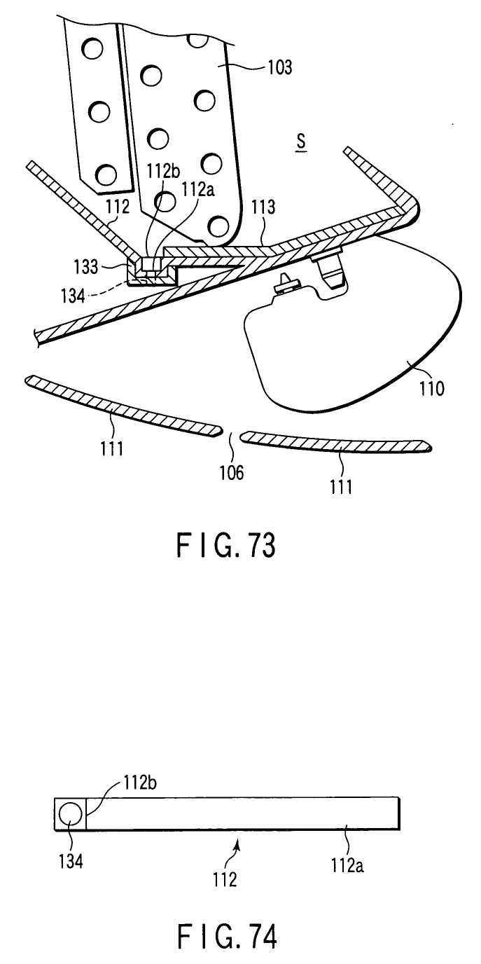

A second embodiment of the inactivating agent activating means will be described below with reference to FIGS. 73 and 74. Condensed water, formed by the cooling operation of the indoor heat exchangers 103, 104 and 105 and pooled in the drain receiver 112, is evaporated by heating with heating means such as an electric heater 133 provided at an appropriate location of the drain receiver 112 to form the high temperature-high humidity atmosphere. The reference numerals 113 and 134 in the drawing denote a heat insulation material and drain holes provided at the bottom of the drain receiver 112, respectively.

Condensed water is formed by the air conditioning and demoisturizing operation and condensed water forming operation described in the first embodiment above and condensed water dripping from the surface of the heat exchanger to the drain receiver 112 is pooled in a recess 112a provided in the drain receiver. This recess 112a is preferably formed as a groove formed on the bottom face of the drain receiver 112 and extending in the direction of width of the air conditioning indoor unit 101. Vapor ascending just above evenly contacts over the entire region of the virus inactivating filter 21. The volume of pooled condensed water in the recess 112a should be enough for making the inner space S high temperature-high humidity for maintaining the heating operation for a required period of time. The volume of pooled condensed water is defined by the cross-sectional configuration and length of the recess 122a as well as by the height of a partition plate 112b provided in the vicinity of the drain hole 134.

The recess 112a is not restricted to be the groove elongating in the direction of width, and various modifications such as recesses divided in the direction of width with a given pitch.

The inner space S having the construction above is heated by flowing an electric current through the electric heater 133 while the inner space S is hermitic or semi-hermetic, as in the heating operation in the first embodiment. Operation of the cross-flow fan 107 is preferably stopped when the inner space S is semi-hermetic, while the inner space S is agitated when the inner space is hermetic. The inactivating agent activating means is formed by adding the electric heater 23 as the heating means to the usual air conditioning indoor unit with a slight modification of the shape of the drain receiver 22.

It is desirable to appropriately adjust the electric heater 133 so as to be able to intermittently flow an electric current for keeping a desired heating operation time, as for operating and halting the compressor 116 and outdoor fan 118 shown in FIG. 69.

As a result, the inner space S is maintained in a high temperature-high humidity atmosphere for activating the inactivating agent, and the virus can be inactivated by aggressively destroying the virus by activating the inactivating agent 24.

Various operations such as the trapping operation and ventilating operation performed before and after the heating operation may be the same as described in the first embodiment.

Since the air conditioning indoor unit and air conditioner comprising the unit is provided with inactivating agent activating means for forming an atmosphere for activating the inactivating agent 24 immobilized on the virus inactivating filter 21, the virus is inactivated by aggressively destroying the virus. An indoor environment having a low virus infection possibility may be provided by reducing the amount of the virus in the room. However, the construction of the invention is not restricted to the embodiments as set forth above, and various modifications are possible within the scope not departing form the spirit of the invention.

The virus inactivating agent of the invention is applicable to protective clothes, medical wears, and paint materials used for wall paper or wall, and for a stretcher and capsule for carrying a patent infected with the virus. The agent is also applicable for spraying on medical wastes and in facilities such as hospital for accommodating patients. Other applications include a door mat, floor mat, carpet and automobile sheet.

1. An allergen inactivating method by maintaining the allergens under a condition in which any one selected from the group consisting of heat, an alkali, an acid and an enzyme exists.

2. The allergen inactivating method according to claim 1, wherein the enzyme is a protease.

3. The allergen inactivating method according to claim 1, by maintaining the allergens under a condition in which the enzyme and a denaturing agent exist.

4. The allergen inactivating method according to claim 3, wherein the denaturing agent is any one of a surfactant, urea and a salt.

5. An allergen inactivating method by maintaining the allergens under a condition in which any one selected from the group consisting of an alkali, an acid and an enzyme, and heat exist.

6. The allergen inactivating method according to claim 3, wherein the enzyme is a protease.

7. The allergen inactivating method according to claim 5, by maintaining the allergens under a condition in which the enzyme and a denaturing agent exist.

8. The allergen inactivating method according to claim 5, wherein the denaturing agent is any one of a surfactant, urea and a salt.

9. An allergen inactivating filter comprising a filter main body and any one of inactivating means of heat, an acid, an alkali and an enzyme having an allergen inactivating function.

10. The allergen inactivating filter according to claim 9, wherein the filter main body comprises a material having any one or both of water absorbing property and moisture absorbing property.

11. The allergen inactivating filter according to claim 9, comprising a carrier between the filter main body and the inactivation means.

12. The allergen inactivating filter according to claim 11, wherein the carrier comprises a material having any one or both of water absorbing property and moisture absorbing property.

13. The allergen inactivating filter according to claim 9, further comprising heating means.

14. The allergen inactivating filter according to claim 9, comprising water feed means in the filter main body.

15. The allergen inactivating filter according to claim 14, wherein the water feed means is a water tank which feeds water to the filter main body by pressurizing means in contact with an edge of the filter main body.

16. An air treating apparatus comprising the allergen inactivating filter according to claim 9.

17. The air treating apparatus according to claim 16, which is any one of an air conditioner, an air cleaner, a dehumidifier and a drier.

18. The air treating apparatus according to claim 16, comprising an allergen removing operation mode.

19. A home electric appliance comprising a guide port which guides air to the inside and a discharge port which discharges air from the inside, wherein the allergen inactivating filter according to claim 9 is provided at the discharge port.

20. A virus inactivating agent containing at least one active component selected from the group consisting of a protein denaturing agent and a protein decomposing enzyme.

21. The virus inactivating agent according to claim 20, containing both the protein denaturing agent and the protein decomposing enzyme.

22. The virus inactivating agent according to claim 20, wherein the enzyme is a protease.

23. A virus inactivating method comprising allowing the virus to contact a solution containing the virus inactivating agent according to claim 20.

24. A virus inactivating filter comprising a virus trapping filter and the virus inactivating agent according to claim 20 adhered to the virus trapping filter.

25. An air conditioning unit comprising an air suction port to suck air, a heat exchanger to cool or heat the air sucked from the suction port by heat-exchange between the air and a coolant, an air blow port to return the air after heat exchange with the heat exchanger into a room, ventilation means for blowing the air sucked from the suction port to be heat-exchanged into the room from the air blow port, a virus inactivating filter which immobilizes the virus inactivating agent according to claim 20 disposed in an inner space through which the air flows, and inactivating agent activating means for maintaining the inner space in an atmosphere in which the virus inactivating agent is activated.

26. The air conditioning unit according to claim 25, wherein the inactivating agent activating means vaporizes condensed water generated by cooling operation of the heat exchanger by heating operation of the heat exchanger executed after the cooling operation.

27. The air conditioning unit according to claim 25, wherein the inactivating agent activating means vaporizes condensed water generated by cooling operation of the heat exchanger and collected in a drain receiver by heating with heating means.

28. The air conditioning unit according to claim 25, wherein, after maintaining the inner space in a high temperature and high humidity state by the inactivating agent activating means, degradation preventive operation of removing moisture from the inactivating agent carrier is executed.

29. The air conditioning unit according to claim 25, wherein, before activating the virus inactivating agent on the inactivating agent carrier, virus trapping operation of sucking indoor air in the inner space to allow the air to flow through the inactivating agent carrier is executed.

30. An air conditioner comprising the air conditioning unit according to claim 25, other conditioning unit having a compressor to compress a coolant and a heat exchanger to heat-exchange between the coolant and air, and piping unit for coolant to circulate the coolant between the said two air conditioning units.
